(19) United States
(12) Patent Application Publication
(10) Pub. No.: US 2008/0064729 A1
(43) Pub. Date: Mar. 13, 2008

(54) Phenethylamide derivatives with kinase inhibitory activity

(75) Inventors: Alexandra E. Gould, Cambridge, MA (US); Paul D. Greenspan, Acton, MA (US); Tricia J. Vos, Medford, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc., Cambridge, MA (US)

(21) Appl. No.: 11/899,361

(22) Filed: Sep. 5, 2007

Related U.S. Application Data
(60) Provisional application No. 60/842,931, filed on Sep. 7, 2006.

(51) Int. Cl. A61K 31/4412; A61K 31/4439; A61P 35/00; C07D 213/68; C07D 401/04; C07D 401/12

(52) U.S. Cl. 514/341; 514/350; 546/274.1; 546/274.7; 546/298

(57) ABSTRACT

The present invention provides novel phenethylamide compounds useful as inhibitors of protein kinases. The invention also provides pharmaceutical compositions comprising the compounds of the invention and methods of using the compositions in the treatment of various diseases.

### RELATED APPLICATIONS

This application claims priority from U.S. Provisional Patent Application Ser. No. 60/842,931, filed on Sep. 7, 2006, which is hereby incorporated by reference in its entirety.

### BACKGROUND OF THE INVENTION

1. Field of the Invention

The present invention relates to protein kinase inhibitors, particularly inhibitors of Raf-kinase. The invention also provides pharmaceutical compositions comprising the compounds of the invention and methods of using the compositions in the treatment of various diseases.

2. Background of the Invention

Protein kinases constitute a large family of structurally related enzymes that effect the transfer of a phosphate group from a nucleoside triphosphate to a Ser, Thr or Tyr residue on a protein acceptor. A vast array of cellular functions, including DNA replication, cell cycle progression, energy metabolism, and cell growth and differentiation, are regulated by reversible protein phosphorylation events mediated by protein kinases. Additionally, protein kinase activity has been implicated in a number of disease states, including cancers. Of the >100 dominant oncogenes known to date, many encode receptor and cytoplasmic tyrosine kinases known to be mutated and/or over expressed in human cancers (Blume-Jensen and Hunter, Nature, 411:355-365 (2001)). Accordingly, protein kinase targets have attracted substantial drug discovery efforts in recent years, with several protein kinase inhibitors achieving regulatory approval (reviewed in Fischer, Curr. Med. Chem., 11:1563 (2004); Dancey and Sausville, Nature Rev. Drug Disc., 2:296 (2003)).

Intracellular signaling pathways activated in response to growth factor/cytokine stimulation are known to control functions such as proliferation, differentiation and cell death (Chiloeches and Marais, In Targets for Cancer Therapy; Transcription Factors and Other Nuclear Proteins, 179-206 (La Thangue and Bandara, eds., Totowa, Humana Press 2002)). One example is the Ras-Raf-MEK-ERK pathway which is controlled by receptor tyrosine kinase activation. Activation of Ras proteins at the cell membrane leads to phosphorylation and recruitment of accessory factors and Raf which is then activated by phosphorylation. Activation of Raf leads to downstream activation of MEK and ERK. ERK has several cytoplasmic and nuclear substrates, including ELK and Ets-family transcription factor, which regulates genes involved in cell growth, survival and migration (Marais et al., J. Biol. Chem., 272:4378-4383 (1997); Peyssonnaux and Eychene, Biol. Cell, 93-53-62 (2001)). As a result, this pathway is an important mediator of tumor cell proliferation and angiogenesis. For instance, overexpression of constitutively active B-Raf can induce an oncogenic event in untransformed cells (Wellbrock et al., Cancer Res., 64:2338-2342 (2004)). Aberrant activation of the pathway, such as by activating Ras and/or Raf mutations, is known to be associated with a malignant phenotype in a variety of tumor types (Bos, Hematol. Pathol., 2:55-63 (1988); Downward, Nature Rev. Cancer, 3:11-22 (2003); Karasarides et al., Oncogene, 23:6292-6298 (2004); Tuveson, Cancer Cell, 4:95-98 (2003); Bos, Cancer Res, 49:4682-4689 (1989)). Activating mutations in B-Raf are found in 60-70% of melanomas. Melanoma cells that carry mutated B-Raf-V599E are transformed, and cell growth, ERK signaling and cell viability are dependent on mutant B-Raf function (Karasarides et al., Oncogene, 23:6292-6298 (2004)). Although this mutation historically has been referred to in the literature as V599E, the mutated valine actually is located at position 600 (Wellbrock et al., Cancer Res., 64:2338-2342 (2004)).

There are three Raf isoforms, A-Raf, B-Raf and C-Raf (Raf-1), all of which can act as downstream effectors of Ras. Although they show significant sequence similarities, they also exhibit distinct roles in development, in addition to significant biochemical and functional differences. In particular, the high basal kinase activity of B-Raf may explain why mutated forms of only this isoform have been found in human cancers. Nevertheless, the isoforms show redundant functions in facilitating oncogenic Ras-induced activation of the MEK-ERK signaling cascade (Wellbrock, Cancer Res, 64:2338-2342 (2004)). In addition to Raf signaling via the MEK-ERK pathway, there is some evidence that C-Raf (and possibly B-Raf and A-Raf) may signal via alternative pathways directly involved in cell survival by interaction with the BH3 family of anti-apoptotic proteins (Wellbrock et al., Nature Rev.: Mol. Cell. Biol., 5:875 (2004)).

Inhibitors of the Raf kinases may be expected to interrupt the Ras-Raf signaling cascade and thereby provide new methods for the treatment of proliferative disorders, such as cancer. There is thus a need for new inhibitors of Raf kinase activity.

### DESCRIPTION OF THE INVENTION

The present invention provides compounds that are effective inhibitors of Raf-kinase. These compounds are useful for inhibiting kinase activity in vitro and in vivo, and are especially useful for the treatment of various cell proliferative diseases.

Compounds useful for the methods of the invention are represented by formula (I): 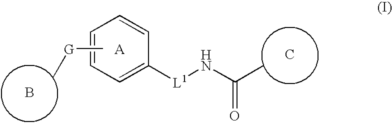
or a pharmaceutically acceptable salt thereof;
wherein:
G is —C(R^{d})(R^{e})—, —O—, —S—, or —N(R^{f})—, wherein G is attached to Ring A at the position meta or para to L¹;
L¹ is —[C(R⁹)(R^{h})]ₘ—C(R^{j})(R^{k})—;
Ring A is substituted with 0-2 R^{aa};
Ring B is a 5- or 6-membered heteroaryl ring having 1-3 ring nitrogen atoms and optionally one additional ring heteroatom selected from oxygen and sulfur;
   Ring B is substituted on its substitutable ring carbon atoms with 0-2 R^{bb} and 0-2 R^{8b};
      each R^{bb} independently is halo, —NO₂, —CN, —C(R⁵)═C(R⁵)₂, —C≡C—R⁵, —OR⁵, —SR⁶, —S(O)R⁶, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OC(O)N(R⁴)₂, —C(O)R⁵, —CO₂R⁷, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR¹)—N(R⁴)₂, —C(═NR⁴)—OR⁵, —C(═NR⁴)—N(R⁴)—OR⁵, —C(R⁶)═N—OR⁵, or an optionally substituted aliphatic, heteroaryl, or heterocyclyl;
      each R^{8b} independently is selected from the group consisting of Cl₄ aliphatic, C₁₋₄ fluoroaliphatic, halo, —OH, —O(C₁₋₄ aliphatic), —NH₂, —NH(C₁₋₄ alkyl), and —N(C₁₋₄ alkyl)₂;
   each substitutable ring nitrogen atom in Ring B is unsubstituted or is substituted with —C(O)R⁵, —C(O)N(R⁴)₂, —CO₂R⁶, —SO₂R⁶, —SO₂N(R⁴)₂, C₁₋₄ aliphatic, an optionally substituted C₆₋₁₀ aryl, or a C₆₋₁₀ ar(C₁₋₄)alkyl, the aryl portion of which is optionally substituted;
   one ring nitrogen atom in Ring B optionally is oxidized;
Ring C is a 5- or 6-membered aryl or heteroaryl ring having 0-3 ring nitrogen atoms and optionally one additional ring heteroatom selected from oxygen and sulfur;
   Ring C is substituted on its substitutable ring carbon atoms with 0-2 R^{cc} and 0-2 R^{8c};
      each R^{cc} independently is halo, —NO₂, —CN, —C(R⁵)═C(R⁵)₂, —C≡C—R⁵, —OR⁵, —SR⁶, —S(O)R⁶, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR¹)—N(R⁴)₂, —C(═NR⁴)—OR⁵, —C(═NR⁴)—N(R⁴)—OR⁵, —C(R⁶)═N—OR⁵, or an optionally substituted aliphatic, aryl, heteroaryl, or heterocyclyl; or two adjacent R^{cc}, taken together with the intervening ring atoms, form a fused Ring E;
      each R^{8c} independently is selected from the group consisting of C₁₋₄ aliphatic, C₁₋₁₄ fluoroaliphatic, —O(C₁₋₄ alkyl), —O(C₁₋₄ fluoroalkyl), and halo;
   each substitutable ring nitrogen atom in Ring C is unsubstituted or is substituted with —C(O)R⁵, —C(O)N(R⁴)₂, —CO₂R⁶, —SO₂R⁶, —SO₂N(R⁴)₂, an optionally substituted C₆₋₁₀ aryl, or a C₁₋₄ aliphatic optionally substituted with —F, —OH, —O(C₁₋₄ alkyl), —CN, —N(R⁴)₂, —C(O)(C₁₋₄ alkyl), —CO₂H, —CO₂(C₁₋₄ alkyl), —C(O)NH₂, —C(O)NH(C₁₋₄ alkyl), or an optionally substituted C₆₋₁₀ aryl ring;
   one ring nitrogen atom in Ring C optionally is oxidized;
Ring E is a 5- or 6-membered aromatic or non-aromatic ring having 0-3 ring heteroatoms independently selected from the group consisting of O, N, and S; each substitutable saturated ring carbon atom in Ring E is unsubstituted or is substituted with ═O, ═S, ═C(R⁵)₂, or —R^{ee};
   each substitutable unsaturated ring carbon atom in Ring E is unsubstituted or is substituted with —R^{ee};
   each R^{ee} independently is halo, —NO₂, —CN, —C(R⁵)═C(R⁵)₂, —C≡C—R⁷, —OR⁵, —SR⁶, —S(O)R⁶, —S₂R⁶, —SO₂N(R⁴)₂—N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —C(═NR⁴)—N(R⁴)—OR⁵, —C(R⁶)═N—OR⁵, or an optionally substituted C₁₋₄ aliphatic;
   each substitutable ring nitrogen atom in Ring E is unsubstituted or is substituted with —C(O)R⁵, —C(O)N(R⁴)₂, —CO₂R⁶, —SO₂R⁶, —SO₂N(R⁴)₂, C₁₋₄ aliphatic, an optionally substituted C₆₋₁₀ aryl, or a C₁₋₁₀ ar(C₁₋₄)alkyl, the aryl portion of which is optionally substituted;
   one ring nitrogen or sulfur atom in Ring E optionally is oxidized;
R^{aa} is halo, —NO₂, —CN, —OR⁵, —SR⁶, —S(O)R⁶, —SO₂R⁶, —SO₂N(R⁴)₂, —N(R⁴)₂, —OC(O)R⁵¹—CO₂R⁵—C(O)N(R⁴)₂, —N(R⁴)SO₂R⁶, —N(R⁴)SO₂N(R⁴)₂, or a C₁₋₄ aliphatic or C₁₋₄ fluoroaliphatic optionally substituted with —OR⁵ or —N(R⁴)₂, provided that no more than one R^{aa} is —OH;
R^{d} is hydrogen, fluoro, C₁₋₄ aliphatic, C₁₋₄ fluoroaliphatic, —NH₂, —NH(C₁₋₄ alkyl), —N(C₁₋₄ alkyl)₂, OH, or —O(C₁₋₄ alkyl);
R⁴ is hydrogen, fluoro, C₁₋₄ aliphatic, or C₁₋₄ fluoroaliphatic; or R^{d} and R^{e}, taken together with the carbon atom to which they are attached, form a 3- to 6-membered cycloaliphatic or heterocyclyl ring;
R^{f} is —H, —C(O)R⁵, —C(O)N(R⁴)₂, —CO₂R⁶—SO₂R⁶, —SO₂N(R⁴)₂, or an optionally substituted C₁₋₆ aliphatic;
R^{g} is hydrogen, fluoro, C₁₋₄ aliphatic, or C₁₋₄ fluoroaliphatic, and R^{h} is hydrogen, fluoro, C₁₋₄ aliphatic, C₁₋₄ fluoroaliphatic, —OH, —O(C₁₋₄ alkyl), —N(R⁴)₂, —N(R⁴)C(O)(C₁₋₄ aliphatic);
or R^{g} and R^{h}, taken together with the carbon atom to which they are attached, form a 3- to 6-membered cycloaliphatic ring;
R^{j} is hydrogen, fluoro, C₁₋₄ aliphatic, or C₁₋₄ fluoroaliphatic, and R^{k} is hydrogen, fluoro, C₁₋₄ aliphatic, C₁₋₄ fluoroaliphatic, —C(O)(C₁₋₄ alkyl), —CO₂H, or —CO₂(C₁₋₄ alkyl); or R^{k} and R^{k}, taken together with the carbon atom to which they are attached, form a 3- to 6 membered cycloaliphatic ring; or
R^{g} and R^{j} are each hydrogen, fluoro, C₁₋₄ aliphatic, or C₁₋₄ fluoroaliphatic, and R^{k} and the vicinal R^{h}, taken together with the intervening carbon atoms, form a 3- to 6-membered cycloaliphatic ring;
each R⁴ independently is hydrogen or an optionally substituted aliphatic, aryl, heteroaryl, or heterocyclyl group; or two R⁴ on the same nitrogen atom, taken together with the nitrogen atom, form an optionally substituted 4 to 8-membered heterocyclyl ring having, in addition to the nitrogen atom, 0-2 ring heteroatoms independently selected from N, O, and S;
each R⁵ independently is hydrogen or an optionally substituted aliphatic, aryl, heteroaryl, or heterocyclyl group; and
each R⁶ independently is an optionally substituted aliphatic, aryl, or heteroaryl group; and
m is 1 or 2.

Compounds of this invention include those described generally above, and are further illustrated by the classes, subclasses, and species disclosed herein. Terms used herein shall be accorded the following defined meanings, unless otherwise indicated.

The terms “Raf” and “Raf kinase” are used interchangeably, and unless otherwise specified refer to any member of the Raf family of kinase enzymes, including without limitation, the isoforms A-Raf, B-Raf, and C-Raf. These enzymes, and the corresponding genes, also may be referred to in the literature by variants of these terms, e.g., RAF, raf, BRAF, B-raf, b-raf. The isoform C-Raf also is referred to by the terms Raf-1 and C-Raf-1.

The term “aliphatic” or “aliphatic group”, as used herein, means a substituted or unsubstituted straight-chain, branched, or cyclic C₁₋₁₂ hydrocarbon, which is completely saturated or which contains one or more units of unsaturation, but which is not aromatic. For example, suitable aliphatic groups include substituted or unsubstituted linear, branched or cyclic alkyl, alkenyl, or alkynyl groups and hybrids thereof, such as (cycloalkyl)alkyl, (cycloalkenyl)alkyl or (cycloalkyl)alkenyl. In various embodiments, the aliphatic group has 1 to 12, 1 to 8, 1 to 6, 1 to 4, or 1 to 3 carbons.

The terms “alkyl”, “alkenyl”, and “alkynyl”, used alone or as part of a larger moiety, refer to a straight or branched chain aliphatic group having from 1 to 12 carbon atoms. For purposes of the present invention, the term “alkyl” will be used when the carbon atom attaching the aliphatic group to the rest of the molecule is a saturated carbon atom. However, an alkyl group may include unsaturation at other carbon atoms. Thus, alkyl groups include, without limitation, methyl, ethyl, propyl, allyl, propargyl, butyl, pentyl, and hexyl.

For purposes of the present invention, the term “alkenyl” will be used when the carbon atom attaching the aliphatic group to the rest of the molecule forms part of a carbon-carbon double bond. Alkenyl groups include, without limitation, vinyl, 1-propenyl, 1-butenyl, 1-pentenyl, and 1-hexenyl.

For purposes of the present invention, the term “alkynyl” will be used when the carbon atom attaching the aliphatic group to the rest of the molecule forms part of a carbon-carbon triple bond. Alkynyl groups include, without limitation, ethynyl, 1-propynyl, 1-butynyl, 1-pentynyl, and 1-hexynyl.

The term “cycloaliphatic”, used alone or as part of a larger moiety, refers to a saturated or partially unsaturated cyclic aliphatic ring system having from 3 to about 14 members, wherein the aliphatic ring system is optionally substituted. In some embodiments, the cycloaliphatic is a monocyclic hydrocarbon having 3-8 or 3-6 ring carbon atoms. Nonlimiting examples include cyclopropyl, cyclobutyl, cyclopentyl, cyclopentenyl, cyclohexyl, cyclohexenyl, cycloheptyl, cycloheptenyl, cyclooctyl, cyclooctenyl, and cyclooctadienyl. In some embodiments, the cycloaliphatic is a bridged or fused bicyclic hydrocarbon having 6-12, 6-10, or 6-8 ring carbon atoms, wherein any individual ring in the bicyclic ring system has 3-8 members.

In some embodiments, two adjacent substituents on the cycloaliphatic ring, taken together with the intervening ring atoms, form an optionally substituted fused 5- to 6-membered aromatic or 3- to 8-membered non-aromatic ring having 0-3 ring heteroatoms selected from the group consisting of O, N, and S. Thus, the term “cycloaliphatic” includes aliphatic rings that are fused to one or more aryl, heteroaryl, or heterocyclyl rings. Nonlimiting examples include indanyl, 5,6,7,8-tetrahydroquinoxalinyl, decahydronaphthyl, or tetrahydronaphthyl, where the radical or point of attachment is on the aliphatic ring.

The terms “aryl” and “ar-”, used alone or as part of a larger moiety, e.g., “aralkyl”, “aralkoxy”, or “aryloxyalkyl”, refer to a C₆ to C₁₄ aromatic hydrocarbon, comprising one to three rings, each of which is optionally substituted. Preferably, the aryl group is a C₆₋₁₀ aryl group. Aryl groups include, without limitation, phenyl, naphthyl, and anthracenyl. In some embodiments, two adjacent substituents on the aryl ring, taken together with the intervening ring atoms, form an optionally substituted fused 5- to 6-membered aromatic or 4- to 8-membered non-aromatic ring having 0-3 ring heteroatoms selected from the group consisting of O, N, and S. Thus, the term “aryl”, as used herein, includes groups in which an aryl ring is fused to one or more heteroaryl, cycloaliphatic, or heterocyclyl rings, where the radical or point of attachment is on the aromatic ring. Nonlimiting examples of such fused ring systems include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, carbazolyl, acridinyl, phenazinyl, phenothiazinyl, phenoxazinyl, tetrahydroquinolinyl, tetrahydroisoquinolinyl, fluorenyl, indanyl, phenanthridinyl, tetrahydronaphthyl, indolinyl, phenoxazinyl, benzodioxanyl, and benzodioxolyl. An aryl group may be mono-, bi-, tri-, or polycyclic, preferably mono-, bi-, or tricyclic, more preferably mono- or bicyclic. The term “aryl” may be used interchangeably with the terms “aryl group”, “aryl moiety”, and “aryl ring”.

An “aralkyl” or “arylalkyl” group comprises an aryl group covalently attached to an alkyl group, either of which independently is optionally substituted. Preferably, the aralkyl group is C₆₋₁₀ aryl(C₁₋₆)alkyl, C₆₋₁₀ aryl(C₁₋₄)alkyl, or C₆₋₁₀ aryl(C₁₋₃)alkyl, including, without limitation, benzyl, phenethyl, and naphthylmethyl.

The terms “heteroaryl” and “heteroar-”, used alone or as part of a larger moiety, e.g., heteroaralkyl, or “heteroaralkoxy”, refer to groups having 5 to 14 ring atoms, preferably 5, 6, 9, or 10 ring atoms; having 6, 10, or 14 n electrons shared in a cyclic array; and having, in addition to carbon atoms, from one to four heteroatoms. The term “heteroatom” refers to nitrogen, oxygen, or sulfur, and includes any oxidized form of nitrogen or sulfur, and any quaternized form of a basic nitrogen. Thus, when used in reference to a ring atom of a heteroaryl, the term “nitrogen” includes an oxidized nitrogen (as in pyridine N-oxide). Certain nitrogen atoms of 5-membered heteroaryl groups also are substitutable, as further defined below. Heteroaryl groups include, without limitation, radicals derived from thiophene, furan, pyrrole, imidazole, pyrazole, triazole, tetrazole, oxazole, isoxazole, oxadiazole, thiazole, isothiazole, thiadiazole, pyridine, pyridazine, pyrimidine, pyrazine, indolizine, naphthyridine, pteridine, pyrrolopyridine, imidazopyridine, oxazolopyridine, thiazolopyridine, triazolopyridine, pyrrolopyrimidine, purine, and triazolopyrimidine. As used herein, the phrase “radical derived from” means a monovalent radical produced by removal of a hydrogen radical from the parent heteroaromatic ring system. Unless otherwise stated, the radical (i.e., the point of attachment of the heteroaryl to the rest of the molecule) may be created at any substitutable position on any ring of the parent heteroaryl ring system.

In some embodiments, two adjacent substituents on the heteroaryl, taken together with the intervening ring atoms, form an optionally substituted fused 5- to 6-membered aromatic or 4- to 8-membered non-aromatic ring having 0-3 ring heteroatoms selected from the group consisting of O, N, and S. Thus, the terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, benzoxazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetraiydroquinolinyl, tetrahydroisoquinolinyl, and pyrido[2,3-b]-1,4-oxazin-3(4H)-one. A heteroaryl group may be mono-, bi-, tri-, or polycyclic, preferably mono-, bi-, or tricyclic, more preferably mono- or bicyclic. The term “heteroaryl” may be used interchangeably with the terms “heteroaryl ring”, or “heteroaryl group”, any of which terms include rings that are optionally substituted. The term “heteroaralkyl” refers to an alkyl group substituted by a heteroaryl, wherein the alkyl and heteroaryl portions independently are optionally substituted.

As used herein, the terms “aromatic ring” and “aromatic ring system” refer to an optionally substituted mono-, bi-, or tricyclic group having 0-6, preferably 0-4 ring heteroatoms, and having 6, 10, or 14 n electrons shared in a cyclic array. Thus, the terms “aromatic ring” and “aromatic ring system” encompass both aryl and heteroaryl groups.

As used herein, the terms “heterocycle”, “heterocyclyl”, “heterocyclic radical”, and “heterocyclic ring” are used interchangeably and refer to a stable 3- to 7-membered monocyclic, or to a fused 7- to 10-membered or bridged 6- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a heterocyclyl ring having 1-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure, and any of the ring atoms can be optionally substituted. Examples of such saturated or partially unsaturated heterocyclic radicals include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl.

In some embodiments, two adjacent substituents on a heterocyclic ring, taken together with the intervening ring atoms, form an optionally substituted fused 5- to 6-membered aromatic or 3- to 8-membered non-aromatic ring having 0-3 ring heteroatoms selected from the group consisting of O, N, and S. Thus, the terms “heterocycle”, “heterocyclyl”, “heterocyclyl ring”, “heterocyclic group”, “heterocyclic moiety”, and “heterocyclic radical”, are used interchangeably herein, and include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono-, bi-, tri-, or polycyclic, preferably mono-, bi-, or tricyclic, more preferably mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond between ring atoms. The term “partially unsaturated” is intended to encompass rings having multiple sites of unsaturation, but is not intended to include aryl or heteroaryl moieties, as herein defined.

The terms “haloaliphatic”, “haloalkyl”, “haloalkenyl” and “haloalkoxy” refer to an aliphatic, alkyl, alkenyl or alkoxy group, as the case may be, which is substituted with one or more halogen atoms. As used herein, the term “halogen” or “halo” means F, Cl, Br, or I. The term “fluoroaliphatic” refers to a haloaliphatic wherein the halogen is fluoro, including perfluorinated aliphatic groups. Examples of fluoroaliphatic groups include, without limitation, fluoromethyl, difluoromethyl, trifluoromethyl, 2-fluoroethyl, 2,2,2-trifluoroethyl, 1,1,2-trifluoroethyl, 1,2,2-trifluoroethyl, and pentafluoroethyl.

The term “linker group” or “linker” means an organic moiety that connects two parts of a compound. Linkers typically comprise an atom such as oxygen or sulfur, a unit such as —NH—, —CH₂—, —C(O)—, —C(O)NH—, or a chain of atoms, such as an alkylene chain. The molecular mass of a linker is typically in the range of about 14 to 200, preferably in the range of 14 to 96 with a length of up to about six atoms. In some embodiments, the linker is a C₁₋₆ alkylene chain.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms is replaced with a substituent. Suitable substituents include those described below for a substituted aliphatic group. An alkylene chain also may be substituted at one or more positions with an aliphatic group or a substituted aliphatic group.

An alkylene chain also can be optionally interrupted by a functional group. An alkylene chain is “interrupted” by a functional group when an internal methylene unit is replaced with the functional group. Examples of suitable “interrupting functional groups” include —C(R*)═C(R*)—, —C≡C—, —O—, —S—, —S(O)—, —S(O)₂—, —S(O)₂N(R⁺)—, —N(R*)—, —N(R⁺)CO—, —N(R⁺)C(O)N(R⁺)—, —N(R⁺)C(═NR⁺)—N(R⁺)—, —N(R⁺)—C(═NR⁺)—, —N(R⁺)CO₂—, —N(R⁺)SO₂—, —N(R⁺)SO₂N(R⁺)—, —OC(O)—, —OC(O)O—, —OC(O)N(R⁺)—, —C(O)—, —CO₂—, —C(O)N(R⁺)—, —C(O)—C(O)—, —C(═NR⁺)—N(R⁺)—, —C(NR⁺)═N—, —C(═NR⁺)—O—, —C(OR*)═N—, —C(R^{o})═N—O—, or —N(R⁺)—N(R⁺)—. Each R⁺, independently, is hydrogen or an optionally substituted aliphatic, aryl, heteroaryl, or heterocyclyl group, or two R⁺ on the same nitrogen atom, taken together with the nitrogen atom, form a 5-8 membered aromatic or non-aromatic ring having, in addition to the nitrogen atom, 0-2 ring heteroatoms selected from N, O, and S. Each R* independently is hydrogen or an optionally substituted aliphatic, aryl, heteroaryl, or heterocyclyl group.

Examples of C₃₋₆ alkylene chains that have been “interrupted” with —O— include —CH₂OCH₂—, —CH₂O(CH₂)₂—, —CH₂O(CH₂)₃—, —CH₂O(CH₂)₄—, —(CH₂)₂OCH₂—, —(CH₂)₂O(CH₂)₂—, —(CH₂)₂O(CH₂)₃—, —(CH₂)₃O(CH₂)—, —(CH₂)₃O(CH₂)₂—, and —(CH₂)₄O(CH₂)—. Other examples of alkylene chains that are “interrupted” with functional groups include —CH₂ZCH₂—, —CH₂Z(CH₂)₂—, —CH₂Z(CH₂)₃—, —CH₂Z(CH₂)₄—, —(CH₂)₂ZCH₂—, —(CH₂)₂Z(CH₂)₂—, —(CH₂)₂Z(CH₂)₃—, —(CH₂)₃Z(CH₂)—, —(CH₂)₃Z(CH₂)₂—, and —(CH₂)₄Z(CH₂)—, wherein Z is one of the “interrupting functional groups” listed above.

For purposes of clarity, all bivalent groups described herein, including, e.g., the alkylene chain linkers described above and the variables G, L¹, T¹, T², T³, T⁴, V¹, and V³, are intended to be read from left to right, with a corresponding left-to-right reading of the formula or structure in which the variable appears.

One of ordinary skill in the art will recognize that when an alkylene chain having an interruption is attached to a functional group, certain combinations are not sufficiently stable for pharmaceutical use. Similarly, certain combinations of V¹, T¹ and R^{2b}, and certain combinations of V³, T³, and R^{2d} would not be sufficiently stable for pharmaceutical use. Only stable or chemically feasible compounds are within the scope of the present invention. A stable or chemically feasible compound is one in which the chemical structure is not substantially altered when kept at a temperature from about −80° C. to about +40° C., preferably −20° C. to about +40° C., in the absence of moisture or other chemically reactive conditions, for at least a week, or a compound which maintains its integrity long enough to be useful for therapeutic or prophylactic administration to a patient.

The term “substituted”, as used herein, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which can be replaced with the radical of a suitable substituent.

The phrase “one or more substituents”, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different.

As used herein, the term “independently selected” means that the same or different values may be selected for multiple instances of a given variable in a single compound. By way of example, in a compound of formula (I), if Ring B is substituted with two substituents —R^{bb}, each substituent is selected from the group of defined values for R^{bb}, and the two values selected may be the same or different.

An aryl (including the aryl moiety in aralkyl, aralkoxy, aryloxyalkyl and the like) or heteroaryl (including the heteroaryl moiety in heteroaralkyl and heteroaralkoxy and the like) group may contain one or more substituents. Examples of suitable substituents on the unsaturated carbon atom of an aryl or heteroaryl group include -halo, —NO₂, —CN, —R*, —C(R*)═C(R*)₂, —C≡C—R*, —OR*, —SR^{o}, —S(O)R^{o}, —SO₂R^{o}, —SO₃R*, —SO₂N(R⁺)₂, —N(R⁺)₂, —NR⁺C(O)R*, —NR⁺C(O)N(R⁺)₂, —N(R⁺)C(═NR⁺)—N(R⁺)₂, —N(R⁺)C(═NR⁺)—R^{o}, —NR⁺CO₂R^{o}, —NR⁺SO₂R^{o}, —NR⁺SO₂N(R⁺)₂, O—C(O)R*, —O—CO₂R*, —OC(O)N(R⁺)₂, —C(O)R*, —CO₂R*, —C(O)—C(O)R*, C(O)N(R⁺)₂, —C(O)N(R⁺)—OR*, —C(O)N(R⁺)C(═NR⁺)—N(R⁺)₂, —N(R⁺)C(═NR⁺)—N(R⁺)—C(O)R*, —C(═NR⁺)—N(R⁺)₂, —C(═NR⁺)—OR*, —N(R⁺)—N(R⁺)₂, —N(R⁺)—OR*, —C(═NR⁺)—N(R⁺)—OR*, —C(R^{o})═N—OR*, —P(O)(R*)₂, —P(O)(OR*)₂, —O—P(O)—OR*, and —P(O)(NR⁺)—N(R⁺)₂, wherein R^{o} is an optionally substituted aliphatic, aryl, or heteroaryl group, and R⁺ and R* are as defined above, or two adjacent substituents, taken together with their intervening atoms, form a 5-6 membered unsaturated or partially unsaturated ring having 0-3 ring atoms selected from the group consisting of N, O, and S.

An aliphatic group or a non-aromatic heterocyclic ring may be substituted with one or more substituents. Examples of suitable substituents on the saturated carbon of an aliphatic group or of a non-aromatic heterocyclic ring include, without limitation, those listed above for the unsaturated carbon of an aryl or heteroaryl group and the following: ═O, ═S, ═C(R*)₂, ═N—N(R*)₂, ═N—OR*, ═N—NHC(O)R^{o}, ═N—NHCO₂R^{o}, ═N—NHSO₂R^{o}, or ═N—R*, where each R* and R^{o} is as defined above. Additionally, two substituents on the same carbon atom, taken together with the carbon atom to which they are attached may form an optionally substituted spirocyclic 3- to 6-membered cycloaliphatic ring.

Suitable substituents on a substitutable nitrogen atom of a heteroaryl or non-aromatic heterocyclic ring include —R*, —N(R*)₂, —C(O)R*, —CO₂R*, —C(O)—C(O)R* —C(O)CH₂C(O)R*, —SO₂R*, —SO₂N(R*)₂, —C(═S)N(R*)₂, —C(═NH)—N(R*)₂, and —NR*SO₂R*; wherein each R* is as defined above. A ring nitrogen atom of a heteroaryl or non-aromatic heterocyclic ring also may be oxidized to form the corresponding N-hydroxy or N-oxide compound. A nonlimiting example of such a heteroaryl having an oxidized ring nitrogen atom is N-oxidopyridyl.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

As used herein, the term “comprises” means “includes, but is not limited to.”

It will be apparent to one skilled in the art that certain compounds of this invention may exist in tautomeric forms, all such tautomeric forms of the compounds being within the scope of the invention. Unless otherwise stated, structures depicted herein are also meant to include all geometric (or conformational) isomers, i.e., (Z) and (E) double bond isomers and (Z) and (E) conformational isomers, as well as all stereochemical forms of the structure; i.e., the R and S configurations for each asymmetric center. Therefore, single stereochemical isomers as well as enantiomeric and diastereomeric mixtures of the present compounds are within the scope of the invention. When a mixture is enriched in one stereoisomer relative to another stereoisomer, the mixture may contain, for example, an enantiomeric excess of at least 50%, 75%, 90%, 99%, or 99.5%.

Unless otherwise stated, structures depicted herein are also meant to include compounds which differ only in the presence of one or more isotopically enriched atoms. For example, compounds having the present structure except for the replacement of a hydrogen atom by a deuterium or tritium, the replacement of a nitrogen atom by an ¹⁵N-enriched nitrogen, or the replacement of a carbon atom by a ¹³C— or ¹⁴C-enriched carbon are within the scope of the invention.

In the compounds of formula (I), Ring A is additionally substituted with 0, 1, or 2 substituents R^{aa}, where R^{aa} is as defined above. Preferably, each R^{aa} independently is selected from the group consisting of halo, C₁₋₄ aliphatic, C₁₋₄ fluoroaliphatic, —NO₂, —CN, —CO₂H, —O(C₁₋₄ alkyl), —O(C₁₋₄ fluoroalkyl), —S(C₁₋₄ alkyl), —SO₂(C₁₋₄ alkyl), —NH₂, —NH(C₁₋₄ alkyl), —N(C₁₋₄ alkyl)₂, —C(O)NH₂, —C(O)NH(C₁₋₄ alkyl), and —C(O)N(C₁₋₄ alkyl)₂. More preferably, each R^{aa} independently is selected from the group consisting of —F, —Cl, —CN, —NO₂, C₁₋₄ alkyl, —CF₃, —O(C₁₋₄ alkyl), —OCF₃, —S(C₁₋₄ alkyl), —SO₂(C₁₋₄ alkyl), —NH₂, —NH(C₁₋₄ alkyl), —N(C₁₋₄ alkyl)₂, —CO₂H, —C(O)NH₂, and —C(O)NH(C₁₋₄ alkyl). In certain embodiments, each R⁴ independently is selected from the group consisting of, —F, —Cl, —NO₂, —CH₃, —CF₃, —OCH₃, —OCF₃, —SCH₃, —SO₂CH₃, —CN, —CO₂H, —C(O)NH₂, and —C(O)NHCH₃. In certain preferred embodiments Ring A has no substituents R^{aa}.

The linker L¹ is a two- or three-carbon alkylene chain having the formula —[C(R^{g})(R^{h})]ₘ—C(R^{j})(R^{k})—, where each of R^{g}, R^{h}, Rⁱ, R^{k}, and m is as defined above. In some embodiments, R^{h} and R^{k} are each independently selected from the group consisting of hydrogen, fluoro, C₁₋₄ alkyl, or C₁₋₄ fluoroalkyl. In some embodiments, the carbon atoms in L¹ are substituted with 0, 1, or 2, preferably 0 or 1, non-hydrogen substituents. In certain preferred embodiments, L¹ is —CH₂—CH₂— or —CH₂—CH₂—CH₂—. As mentioned above, the bivalent group L¹ is intended to be read from left to right, with the carbon atom bearing R^{g} and R^{h} attached to Ring A, and the carbon atom bearing R^{j} and R^{k} attached to the amide carbonyl.

The linker G is a one-atom linker selected from the group consisting of —C(R^{d})(R^{e})—, —C(O)—, —O—, —S—, —S(O)—, —S(O)₂—, or —N(R^{f})—, where each of R^{d}, R^{e}, and R^{f} is as defined above. The linker G is attached to Ring A at the position that is meta or para to L¹.

When G is a carbon linker, R^{d} and R^{e} preferably are each independently hydrogen, fluoro, C₁₋₄ aliphatic, or C₁₋₄ fluoroaliphatic. Alternatively, R^{d} and R^{e}, taken together with the carbon atom to which they are attached, form a 3- to 6-membered cycloaliphatic or heterocyclyl ring, preferably a cyclopropyl ring. In some embodiments, each of R^{d} and R⁴ is hydrogen. When G is a nitrogen linker, R^{f} preferably is hydrogen, —C(O)R⁵, or an optionally substituted C₁₋₄ aliphatic. More preferably, R^{f} is hydrogen. Most preferably, G is —O— or —NH—.

In some embodiments of the present invention, the compound of formula (I) is characterized by one or more of the following features:
(a) each R^{aa} independently is —F, —Cl, —CN, —NO₂, C₁₋₄ alkyl, —CF₃, —O(C₁₋₄ alkyl), —OCF₃, —S(C₁₋₄ alkyl), —SO₂(C₁₋₄ alkyl), —NH₂, —NH(C₁₋₄ alkyl), —N(C₁₋₄ alkyl)₂, —CO₂H, —C(O)NH₂, or —C(O)NH(C₁₋₄ alkyl);
(b) R^{h} and R^{k} are each independently hydrogen, fluoro, C₁₋₄ alkyl, or C₁₋₄ fluoroalkyl;
(c) L¹ is —CH₂—CH₂— or —CH₂—CH₂—CH₂—; and
(d) G is —O— or —NH—.

In the compounds of formula (I), Ring B is an optionally substituted 5- or 6-membered heteroaryl ring having 1-3 ring nitrogen atoms and optionally one additional ring heteroatom selected from oxygen and sulfur. Each substitutable ring nitrogen atom in Ring B is unsubstituted or substituted, preferably with —C(O)R⁵, —C(O)N(R⁴)₂, —CO₂R⁶, —SO₂R⁶, —SO₂N(R⁴)₂, C₁₋₄ aliphatic, an optionally substituted C₆₋₁₀ aryl, or a C₆₋₁₀ ar(C₁₋₄)alkyl, the aryl portion of which is optionally substituted. One ring nitrogen atom in Ring B optionally is oxidized. In some embodiments, the substitutable ring nitrogen atoms in Ring B all are unsubstituted, and one ring nitrogen atom optionally is oxidized.

In some embodiments, Ring B is a radical derived from an aromatic ring system selected from the group consisting of pyrrole, oxazole, thiazole, imidazole, pyrazole, isoxazole, isothiazole, oxadiazole, triazole, thiadiazole, pyridine, pyridazine, pyrimidine, pyrazine, and triazine. Any such ring system optionally is substituted on any substitutable ring carbon or ring nitrogen atom, and one ring nitrogen atom optionally is oxidized.

Preferably, Ring B is a radical derived from pyrrole, oxazole, thiazole, imidazole, pyrazole, isoxazole, pyridine, pyridazine, or pyrimidine, wherein Ring B optionally is substituted on any substitutable ring carbon or ring nitrogen atom, and one ring nitrogen atom optionally is oxidized. In some embodiments, Ring B is selected from the group consisting of 3-pyridyl, 4-pyridyl, 4-pyridazinyl, 4-pyrimidinyl, 5-pyrimidinyl, 2-oxazolyl, 4-oxazolyl, 5-oxazolyl, 2-thiazolyl, 4-thiazolyl, 5-thiazolyl, 2-imidazolyl, 4-imidazolyl, 5-imidazolyl, 2-pyrrolyl, and 3-pyrrolyl, wherein Ring B optionally is substituted on any substitutable ring carbon atom or ring nitrogen atom, and one ring nitrogen atom optionally is oxidized. In some embodiments, Ring B is other than substituted or unsubstituted imidazolyl when Ring C is substituted or unsubstituted phenyl and G¹ is —CH₂— in the para position. In certain preferred embodiments, Ring B is an optionally substituted 4-pyrimidinyl, 4-pyridyl, or N-oxido-4-pyridyl.

Substitutable ring carbon atoms in Ring B preferably are substituted with 0-2 R^{bb} and 0-2 R^{8b}. Each R^{8b} independently is selected from the group consisting of Cl₄ aliphatic, C₁₋₄ fluoroaliphatic, halo, —OH, —O(C₁₋₄ aliphatic), —NH₂, —NH(C₁₋₄ aliphatic), and —N(C₁₋₄ aliphatic)₂. Each R^{bb} independently is halo, —NO₂, —CN, —C(R⁵)═C(R⁵)₂, —C≡C—R⁵, —R⁵, —SR⁶, —S(O)R⁶, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶—N(R⁴)SO₂R⁶¹—N(R⁴)SO₂N(R⁴)₂, —O—C(O)R⁵, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —C(═NR⁴)—N(R⁴)—OR⁵, —C(R⁶)═N—OR⁵, or an optionally substituted aliphatic, heteroaryl, or heterocyclyl.

In some embodiments, each R^{bb} independently is selected from the group consisting of C₁₋₆ aliphatic, C₁₋₆ fluoroaliphatic, halo, —R^{2b}, -T¹-R^{1b}, -T¹-R^{2b}, —V¹-T¹-R^{1b}, —V¹-T¹-R^{2b}, optionally substituted heteroaryl, and optionally substituted heterocyclyl. The variables T¹, V¹, R^{1b}, and R^{2b} have the values described below.

T¹ is a C₁₋₆ alkylene chain optionally substituted with R^{3a} or R^{3b}, wherein the alkylene chain optionally is interrupted by —C(R⁵)═C(R⁵)—, —C≡C—, —O—, —S, —S(O)—, —S(O)₂—, —SO₂N(R⁴)—, —N(R⁴)—, —N(R⁴)C(O)—, —NR⁴C(O)N(R⁴)—, —N(R⁴)C(═NR⁴)—N(R⁴)—, —N(R⁴)—C(═NR⁴)—, —N(R⁴)CO₂—, —N(R⁴)SO₂—, —N(R⁴)SO₂N(R⁴)—, —OC(O)—, —OC(O)N(R⁴)—, —C(O)—, —CO₂—, —C(O)N(R⁴)—, —C(═NR⁴)—N(R⁴)—, —C(NR⁴)═N(R⁴)—, —C(═NR⁴)—O—, or —C(R⁶)═N—O—, and wherein T or a portion thereof optionally forms part of a 3-7 membered ring. In some embodiments, T¹ is a C₁₋₄ alkylene chain optionally substituted with one or two substituents independently selected from the group consisting of C₁₋₃ aliphatic, C₁₋₃ fluoroaliphatic, —F, —OH, —O(C₁₋₄ alkyl), —CO₂H, —CO₂(C₁₋₄ alkyl), —C(O)NH₂, and —C(O)NH(C₁₋₄ alkyl), wherein the alkylene chain optionally is interrupted with —N(R⁴)—, —C(═NR⁴)—N(R⁴)—, —C(NR⁴)═N(R⁴)—, —N(R⁴)—C(═NR⁴)—, —N(R⁴)—C(O)—, or —C(O)N(R⁴)—. In some particular embodiments, T¹ is a C, 6 or C₁₋₄ alkylene chain optionally substituted with —F, C₁₋₃ alkyl, or C₁₋₃ fluoroalkyl, wherein the alkylene-chain optionally is interrupted by —N(R⁴)—, —C(O)—N(R⁴)—, —C(═NR⁴)—N(R⁴)—, —C(NR⁴)═N(R⁴)—, —N(R⁴)—C(O)—, or —N(R⁴)—C(═NR⁴)—. In certain particular embodiments, T¹ is a C₁₋₄ alkylene chain optionally substituted with —F, C₁₋₃ alkyl, or C₁₋₃ fluoroalkyl.

V¹ is —C(R⁵)═C(R⁵)—, —C≡C—, —O—, —S—, —S(O)—, —S(O)₂—, —SO₂N(R⁴)—, —N(R⁴)—, —N(R⁴)C(O)—, —NR⁴C(O)N(R⁴)—, —N(R⁴)C(═NR⁴)—N(R⁴)—, —N(R⁴)C(═NR⁴)—, —N(R⁴)CO₂—, —N(R⁴)SO₂—, —N(R⁴)SO₂N(R⁴)—, —OC(O)—, —OC(O)N(R⁴)—, —C(O)—, —CO₂—, —C(O)N(R⁴)—, —C(O)N(R⁴)—O—, —C(O)N(R⁴)C(═NR⁴)—N(R⁴)—, —N(R⁴)C(═NR⁴)—N(R⁴)—C(O)—, —C(═NR⁴)—N(R⁴)—, —C(NR⁴)═N(R⁴)—, —C(═NR⁴)—O—, or —C(R⁶)═N—O—. In some embodiments, V¹ is —C(R⁵)═C(R⁵)—, —C≡C—, —O—, —N(R⁴)—, —N(R⁴)C(O)—, —C(O)N(R⁴)—, —C(═NR⁴)—N(R⁴)—, —C(NR⁴)═N(R⁴)—, or —N(R⁴)—C(═NR⁴)—. In certain preferred embodiments, V¹ is —N(R⁴)—, —N(R⁴)—C(O)—, —C(O)N(R⁴)—, —C(═NR⁴)N(R⁴)—, or —N(R⁴)—C(═NR⁴)—. In certain particular embodiments, V¹ is —N(R^{4x})—, —N(R^{4x})—C(O)—, —C(O)N(R^{4x})—, —C(═NR^{4x})N(R^{4x})—, or —N(R^{4x})—C(═NR^{4x})—, where each R^{4x} independently is hydrogen, C₁₋₄ alkyl, C₁₋₄ fluoroalkyl, or C₆₋₁₀ ar(C₁₋₄)alkyl, the aryl portion of which may be optionally substituted. In some embodiments, V¹ is —C(O)NH—, —NH—C(O)—, or —C(═NH)NH—.

Each R^{1b} independently is an optionally substituted aryl, heteroaryl, heterocyclyl, or cycloaliphatic ring. In some embodiments, R^{1b} is an optionally substituted C₃₋₆ cycloaliphatic or an optionally substituted phenyl, azetidinyl, pyrrolyl, imidazolyl, oxazolyl, thiazolyl, isoxazolyl, isothiazolyl, pyrazolyl, triazolyl, tetrazolyl, oxadiazolyl, thiadiazolyl, pyrrolinyl, imidazolinyl, pyrazolinyl, pyrrolidinyl, imidazolidinyl, pyrazolidinyl, piperidinyl, morpholinyl, piperazinyl, pyridyl, pyridazinyl, pyrimidinyl, pyrazinyl, or tetrahydropyrimidinyl. In certain preferred embodiments, R^{1b} is an optionally substituted C₃₋₆ cycloaliphatic or an optionally substituted pyrrolidinyl, piperidinyl, morpholinyl, or piperazinyl ring.

Each R^{b} independently is —NO₂, —CN, —C(R⁵)═C(R⁵)₂, —C≡C—R⁵, —OR⁵, —SR⁶, —S(O)R⁶, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —C(═NR⁴)—N(R⁴)—OR⁵, or —C(R⁶)═N—OR⁵. In some embodiments, each R^{2b} independently is —OR⁵, —N(R⁴)₂, —NR⁴C(O)R⁵, —NR⁴C(O)N(R⁴)₂, —C(O)N(R⁴)—OR⁵, —C(O)N(R⁴)₂, —N(R⁴)—CO₂R⁵, —N(R⁴)—C(═NR⁴)—R⁵ or —C(═NR⁴)—N(R⁴). In some embodiments, each R^{2b} independently is —N(R⁴)₂, —NR⁴C(O)R⁵, —C(O)N(R⁴)₂, —CO₂R⁵, or —OR⁵.

Each R^{3a} independently is selected from the group consisting of —F, —OH, —O(C₁₋₄ alkyl), —CN, —N(R⁴)₂, —C(O)(C₁₋₄ alkyl), —CO₂H, —CO₂(C₁₋₄ alkyl), —C(O)NH₂, and —C(O)NH(C₁₋₄ alkyl).

Each R^{1b} independently is a C₁₋₃ aliphatic optionally substituted with R^{1a} or R⁷, or two substituents R^{1b} on the same carbon atom, taken together with the carbon atom to which they are attached, form a 3- to 6-membered cycloaliphatic ring.

Each R⁴ independently is hydrogen or an optionally substituted aliphatic, aryl, heteroaryl, or heterocyclyl group; or two R⁴ on the same nitrogen atom, taken together with the nitrogen atom, form an optionally substituted 4- to 8-membered heterocyclyl ring having, in addition to the nitrogen atom, 0-2 ring heteroatoms selected from N, O, and S.

Each R⁵ independently is hydrogen or an optionally substituted aliphatic, aryl, heteroaryl, or heterocyclyl group.

Each R⁶ independently is an optionally substituted aliphatic, aryl, or heteroaryl group.

Each R⁷ independently is an optionally substituted aryl or heteroaryl ring.

In some embodiments, the substitutable ring carbon atoms in Ring B are substituted with 0-1 R^{bb} and 0-2 R^{8b}. More preferably, the substitutable ring carbon atoms in Ring B are substituted with 0-1 R^{bb} and 0-1 R^{1b}. In such embodiments, R^{bb} preferably is selected from the group consisting of C₁₋₄ aliphatic, C₁₋₄ fluoroaliphatic, halo, —R^{2b}, -T¹-R^{1b}T¹-R^{2b}, —V¹-T¹-R^{1b}, —V¹-T¹-R^{2b}, optionally substituted heteroaryl, and optionally substituted heterocyclyl, where:
T¹ is a C₁₋₄ alkylene chain optionally substituted with one or two substituents independently selected from the group consisting of C₁₋₃ aliphatic, C₁₋₃ fluoroaliphatic, —F, —OH, —O(C₁₋₄ alkyl), —CO₂—H, —CO₂(C₁₋₄ alkyl), —C(O)NH₂, and —C(O)NH(C₁₋₄ alkyl), wherein the alkylene chain optionally is interrupted with —N(R⁴)—, —C(═NR⁴)—N(R⁴)—, —C(NR⁴)═N(R⁴)—, —N(R⁴)—C(═NR⁴)—, —N(R⁴)—C(O)—, or —C(O)N(R⁴)—;
V¹ is —C(R⁵)═C(R⁵)—, —C≡C—, —O—, —N(R⁴)—, —N(R⁴)C(O)—, —C(O)N(R⁴)—, —C(═NR⁴)—N(R⁴)—, —C(NR⁴)═N(R⁴)—, or —N(R⁴)—C(═NR⁴)—;
each R^{1b} independently is an optionally substituted aryl, heteroaryl, heterocyclyl, or cycloaliphatic ring; and
each R^{2b} independently is —NO₂, —CN, —C(R⁵)═C(R⁵)₂, —C≡C—R⁴, —OR⁵, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶—N(R⁴)SO₂R⁶, —N(R⁴)SO₂N(R⁴)₂, —O—C(O)R⁵, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR¹)—N(R⁴)₂, —C(═NR⁴)—OR⁵, —C(═NR⁴)—N(R⁴)—OR⁵, or —C(R⁶)═N—OR⁵.

In a more particular embodiment, the invention relates to a subgenus of the compounds of formula (I), characterized by formula (II): 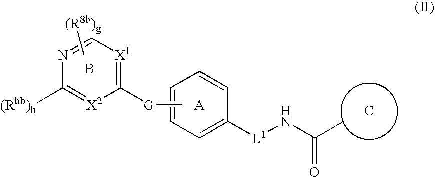
or a pharmaceutically acceptable salt thereof;
wherein:
X¹ and X² are each independently CH or N, provided that X¹ and X² are not both N; one ring nitrogen atom in Ring B optionally is oxidized;
g is 0 or 1;
h is 0 or 1; and

Rings A and C, and the variables L¹, G, R^{bb}, and R^{8b} have the values and preferred values described above for formula (I).

In some embodiments, the invention relates to a compound of formula (II) or a pharmaceutically acceptable salt thereof, wherein R^{bb} is selected from the group consisting of halo, —N(R⁴)₂, —CO₂R⁵, —C(O)—N(R⁴)₂, —C(O)—N(R⁴)—OR⁵, —N(R⁴)C(O)R⁵, —N(R⁴)C(O)—OR⁵, —N(R⁴)C(O)—N(R⁴)₂, —N(R⁴)SO₂R⁶, —C(═NR¹)N(R⁴)₂, and —C(═NR⁴)N(R⁴)—OR⁵. In some embodiments, R^{bb} is —N(R⁴)₂, —C(O)—N(R⁴)₂, —N(R⁴)C(O)R⁵, —C(═NR⁴)N(R⁴)₂, or —C(═NR⁴)N(R⁴)—OR⁵.

In some embodiments, R^{bb} is selected from the group consisting of halo, —N(R^{4x})(R^{4z}), —CO₂ R^{5x}, —C(O)—N(R^{4x})(R^{4z}), —C(O)—N(R^{4x})—OR^{5x}, —N(R^{4x})C(O)R^{5x}, —N(R^{4x})C(O)—OR^{5x}, —N(R^{4x})C(O)—N(R^{4x})(R^{4z}), —N(R^{4x})SO₂R^{6x}, —C(═NR^{4x})N(R^{4x})(R^{4z}), and —C(═N)N(R^{4x})—OR^{5x}. In certain such embodiments, R^{bb} is selected from the group consisting of halo, —NH(R⁴), —N(R^{4x})(R^{4z}), —CO₂R^{4x}, —C(O)—NH(R^{4z}), —C(O)—N(R^{4x})(R^{4z}), —C(O)—NH—OR^{5x}, —NHC(O)R^{5x}, —NHC(O)—OR^{5x}, —NHC(O)—N(R^{4x})(R^{4z}), —NHSO₂R⁶, —C(═NH)N(R^{4x})(R^{4z}), —C(═NH)N(Rex)(R^{4z}), and —C(═NH)NH—OR^{5x}.

In these embodiments, each R^{4x} independently is hydrogen, C₁₋₄ alkyl, C₁₋₄ fluoroalkyl, or C₆₋₁₀ ar(C₁₋₄)alkyl, the aryl portion of which may be optionally substituted, and each R^{4z} independently is hydrogen, C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring; or R^{4x} and R^{4z}, taken together with the nitrogen atom to which they are attached, form an optionally substituted 4 to 8-membered heterocyclyl ring having, in addition to the nitrogen atom, 0-2 ring heteroatoms independently selected from N, O, and S. In some embodiments, R^{4x} and R^{4z}, taken together with the nitrogen atom to which they are attached, form an optionally substituted morpholinyl, piperidinyl, piperazinyl, or pyrrolidinyl ring.

Each R^{5x} independently is hydrogen, C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring.

Each R^{6x} independently is C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring.

In some embodiments, R^{bb} is —N(R^{4x})(R^{4z}), —C(O)—N(R^{4x})(R^{4z}), —N(R^{4x})C(O)R^{5x} or —C(═NH)N(R^{4x})(R^{4z}). In certain such embodiments, R^{4x} and R^{4z}, taken together with the nitrogen atom to which they are attached, form a morpholinyl, piperidinyl, piperazinyl, or pyrrolidinyl ring. In certain other embodiments, R^{bb} is —C(O)—NHCH₃ or —NHC(O)CH₃.

In other embodiments, the invention relates to a compound of formula (II) or a pharmaceutically acceptable salt thereof, wherein R^{bb} is —V¹-T¹-R^{1b} or —V¹-T¹-R^{2b}, where the variables V¹, T¹, R^{1b}, and R^{2b} have the values described below.

V¹ is —N(R⁴)—, —N(R⁴)—C(O)—, —N(R⁴)SO₂R⁶, —N(R⁴)C(O)—OR⁵, —C(O)N(R⁴)—, —C(═NR⁴)N(R⁴)—, or —N(R⁴)—C(═NR¹)—. In some embodiments, V¹ is —N(R^{4x})—, —N(R^{4x})—C(O)—, —C(O)N(R^{4x})—, —C(═NR^{4x})N(R^{4x})—, or —N(R^{4x})—C(═NR^{4x})—, where each R^{4x} independently is hydrogen, C₁₋₄ alkyl, C₁₋₄ fluoroalkyl, or C₆₋₁₀ ar(C₁₋₄)alkyl, the aryl portion of which may be optionally substituted. In some embodiments, V¹ is —C(O)—NH—, —NH—C(O)—, or —C(═NH)NH—.

T¹ is a C₁₋₄ alkylene chain optionally substituted with —F, C₁₋₃ alkyl, or C₁₋₃ fluoroalkyl.

R^{1b} is an optionally substituted C₃₋₆ cycloaliphatic or an optionally substituted phenyl, pyrrolyl, imidazolyl, oxazolyl, thiazolyl, isoxazolyl, isothiazolyl, pyrazolyl, triazolyl, tetrazolyl, oxadiazolyl, thiadiazolyl, pyrrolinyl, imidazolinyl, pyrazolinyl, pyrrolidinyl, imidazolidinyl, pyrazolidinyl, piperidinyl, morpholinyl, piperazinyl, pyridyl, pyridazinyl, pyrimidinyl, pyrazinyl, or tetrahydropyrimidinyl ring. In some embodiments, R^{1b} is an optionally substituted C₃₋₆ cycloaliphatic or an optionally substituted pyrrolidinyl, piperidinyl, morpholinyl, or piperazinyl.

R^{2b} is —N(R⁴)₂, —NR⁴C(O)R⁵, —N(R⁴)C(O)—OR⁵, —N(R⁴)C(O)—N(R⁴)₂, —C(O)N(R⁴)₂, —CO₂R⁵, or —OR⁵. In some embodiments, R^{2b} is —N(R^{4x})(R^{4z}), —NR^{4x}C(O)R^{5x}, —N(R^{4x})C(O)—OR^{5x}, —N(R^{4x})C(O)—N(R^{4x})(R^{4z}), —C(O)N(R^{4x})(R^{4z}), —CO₂R^{5x}, or —OR^{5x}.

In certain such embodiments, R^{bb} is selected from the group consisting of:

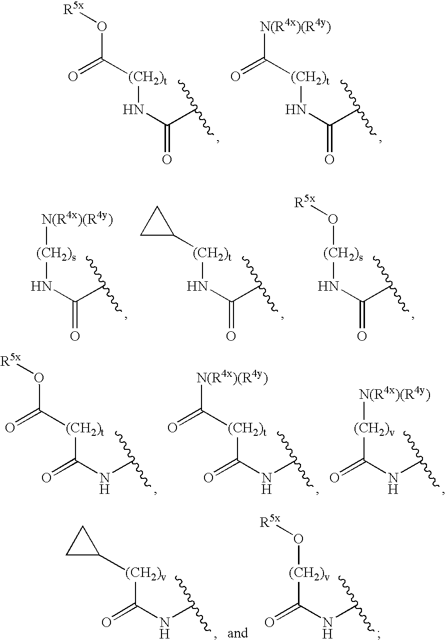

is 2 or 3, t is 1, 2, or 3, and v is 0, 1, 2, or 3.

In some other embodiments, the invention relates to a compound of formula (II) or a pharmaceutically acceptable salt thereof, wherein R^{bb} is -T¹-R^{1b} or -T¹-R^{2b}. T¹ is a C₁₋₆ alkylene chain optionally substituted with —F, C₁₋₃ alkyl, or C₁₋₃ fluoroalkyl, wherein the alkylene chain optionally is interrupted by —N(R⁴)—, —C(O)—N(R⁴)—, —C(═NR⁴)—N(R⁴)—, —C(NR⁴)═N(R⁴)—, —N(R⁴)—C(O)—, or —N(R⁴)—C(═NR⁴)—. R^{1b} is an optionally substituted C₃₋₆ cycloaliphatic or an optionally substituted phenyl, pyrrolyl, imidazolyl, oxazolyl, thiazolyl, isoxazolyl, isothiazolyl, pyrazolyl, triazolyl, tetrazolyl, oxadiazolyl, thiadiazolyl, pyrrolinyl, imidazolinyl, pyrazolinyl, pyrrolidinyl, imidazolidinyl, pyrazolidinyl, piperidinyl, morpholinyl, piperazinyl, pyridyl, pyridazinyl, pyrimidinyl, pyrazinyl, or tetrahydropyrimidinyl ring. R^{2b} is —OR⁵, —N(R⁴)₂, —NR⁴C(O)R⁵, —NR⁴C(O)N(R⁴)₂, —C(O)N(R⁴)—OR⁵, —C(O)N(R⁴)₂, —N(R⁴)—CO₂R⁵, —N(R⁴)—C(═NR⁴)—R⁵ or —C(═NR⁴)—N(R⁴)₂.

In some such embodiments, R^{bb} is selected from the group consisting of —(CH₂)_{q}—R^{1x}, (CH₂)_{q}—R^{2x}, —(CH₂)_{q}—R^{2y}(CH₂)_{q}—N(R^{4x})—(CH₂)_{q}R^{1x}, —(CH₂)_{q}—N(R^{4x})—(CH₂)_{q}—R^{2x}, —(CH₂)_{q}—N(R^{4x})—(CH₂), —R^{2y} —(CH₂)_{q}—N(R^{4x})C(═NR^{4x})—(CH₂)_{q}—R^{1x}, —(CH₂)_{q}—N(R^{4x})C(═NR^{4x})—(CH₂)_{q}—R^{2x}, —(CH₂)_{q}—N(R^{1x})C(═NR^{4x})—(CH₂)_{q}—R^{2y}, wherein q at each occurrence independently is 1, 2, or 3, and s is 2 or 3. R^{1x} is an optionally substituted phenyl, piperidinyl, piperazinyl, morpholinyl, or pyrrolidinyl ring. R^{2x} is —C(O)N(R^{4x})(R^{4z}). R^{2y} is —N(R^{4x})(R^{4z}), —NR^{4x}C(O)R^{5x}, —N(R^{4x})—CO₂R^{5x}, —N(R^{4x})—C(═NR^{4x})—R^{5x} or —OR^{1x}. R^{4x} is hydrogen, C₁₋₄ alkyl, C₁₋₄ fluoroalkyl, or C₆₋₁₀ ar(C₁₋₄)alkyl, the aryl portion of which may be optionally substituted; R^{4z} is hydrogen, C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring; or R^{4x} and R^{4z}, taken together with the nitrogen atom to which they are attached, form an optionally substituted morpholinyl, piperidinyl, piperazinyl, or pyrrolidinyl ring. R^{5x} is hydrogen, C₁₋₄ alkyl, C₁₋₄ fluoroalkyl, or C₆₋₁₀ ar(C₁₋₄)alkyl, the aryl portion of which may be optionally substituted.

Another embodiment of the invention relates to a compound of formula (II) wherein R^{bb} is an optionally substituted heteroaryl or heterocyclyl ring. In such embodiments, the compound has formula (III): 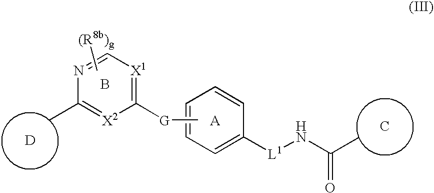
or a pharmaceutically acceptable salt thereof;
wherein:
X¹ and X² are each independently CH or N, provided that X¹ and X² are not both N;
Ring D is an optionally substituted heteroaryl or heterocyclyl ring;
Ring A, Ring C, and the variables R^{b}, G, and L¹ have the values and preferred values described above for formulae (I) or (II); and
g is 0 or 1.

In some embodiments, X¹ and X² are each CH.

Each substitutable ring nitrogen atom in Ring D preferably is unsubstituted or is substituted with —C(O)R⁵, —C(O)N(R⁴)₂, —CO₂R⁶, —SO₂R⁶—SO₂(NR⁴)₂, an optionally substituted C₆₋₁₀ aryl, or a C₁₋₄ aliphatic optionally substituted with R³ or R⁷; and one ring nitrogen atom in Ring D optionally is oxidized.

In some embodiments, Ring D is an optionally substituted heteroaryl or heterocyclyl selected from the group consisting of azetidinyl, pyrrolyl, imidazolyl, oxazolyl, thiazolyl, isoxazolyl, isothiazolyl, pyrazolyl, triazolyl, tetrazolyl, oxadiazolyl, thiadiazolyl, pyrrolinyl, imidazolinyl, pyrazolinyl, pyrrolidinyl, imidazolidinyl, pyrazolidinyl, piperidinyl, morpholinyl, piperazinyl, pyridyl, pyridazinyl, pyrimidinyl, pyrazinyl, and tetrahydropyrimidinyl. In certain embodiments, Ring D is an optionally substituted imidazolyl, oxazolyl, thiazolyl, oxadiazolyl, thiadiazolyl, tetrazolyl, imidazolinyl, or tetrahydropyrimidinyl.

Each substitutable saturated ring carbon atom in Ring D preferably is unsubstituted or is substituted with ═O, ═S, ═C(R⁵)₂, ═N—OR⁵, ═N—R⁵, or —R^{dd}.

Each substitutable unsaturated ring carbon atom in Ring D preferably is unsubstituted or is substituted with −R^{dd}.

Each R^{dd} independently is halo, —NO₂, —CN, —C(R⁵)═C(R⁵)₂, —C≡C—R⁵, —OR⁵, —SR⁶, —S(O)R⁶, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NRCO₂R⁶, —N(R⁴)SO₂R⁶, —N(R⁴)SO₂N(R⁴)₂, —O—C(O)R⁴, —OC(O)N(R⁴)₂, —C(O)R⁵, —CO₂R⁵, —C(O)N(R⁴)₂, —C(O)N(R⁴)—OR⁷, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —C(═NR⁴)—N(R⁴)—OR⁵, —C(R⁶)═N—OR⁵, or an optionally substituted aliphatic, aryl, heteroaryl, or heterocyclyl.

In some embodiments, Ring D is substituted with 0-1 R^{dd} and 0-1 R^{8d}. R^{8d} is C₁₋₄ aliphatic, C₁₋₄ fluoroaliphatic, halo, —OH, —O(C₁₋₄ aliphatic), —NH₂, —NH(C₁₋₄ aliphatic), or —N(C₁₋₄ aliphatic)₂. R^{dd} is selected from the group consisting of C₁₋₄ aliphatic, C₁₋₄ fluoroaliphatic, halo, —R^{1d}, —R^{2d}, -T³-R^{1d}, -T³-R^{2d}, —V³-T³-R^{1d}, and —V³-T³-R^{2d}. The variables T³, V³, R^{1d}, and R^{2d} have the values described below.

T³ is a C₁₋₄ alkylene chain optionally substituted with one or two substituents independently selected from the group consisting of C₁₋₃ aliphatic, C₁₋₃ fluoroaliphatic, —F, —OH, —O(C₁₋₄ alkyl), —CO₂H, —CO₂(C₁₋₄ alkyl), —C(O)NH₂, and —C(O)NH(C₁₋₄ alkyl). In some embodiments, T³ is —(CH₂)— or —(CH₂)₂—.

V³ is —O—, —N(R⁴)—, —N(R⁴)C(O)—, —C(O)N(R⁴)—, —C(═NR⁴)—N(R⁴)—, —C(NR⁴)═N(R⁴)—, or —N(R⁴)C(═NR⁴)—.

Each R^{1d} independently is an optionally substituted aryl, heteroaryl, heterocyclyl, or cycloaliphatic ring. In some embodiments, R^{1d} is an optionally substituted phenyl, pyridyl, or pyrimidinyl group.

Each R^{2d} independently is —NO₂, —CN, —C(R⁵)═C(R⁵)₂, —C≡C—R⁵—OR⁵, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵—OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —C(═NR⁴)—N(R⁴)—OR⁵, or —C(R⁶)═N—OR⁵. In some embodiments, each R^{2d} independently is selected from the group consisting of —OR⁵, —N(R⁴)₂, —NR⁴C(O)R⁵, —NR⁴C(O)N(R⁴)₂, —O—C(O)R⁵, —CO₂R⁵, —C(O)R⁵, —C(O)N(R⁴)₂, —C(O)N(R⁴)—OR⁵, and —C(═NR⁴)—N(R⁴)₂. In some embodiments, each R^{2d} is selected from the group consisting of —OR⁷, —N(R⁴)₂, —CO₂R⁵, or —C(O)N(R⁴)₂.

In some embodiments, Ring D is selected from the group consisting of: 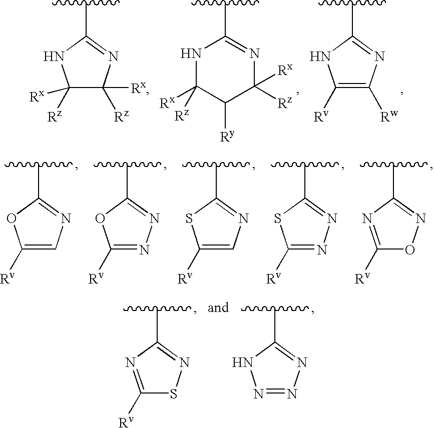
where R^{v}, R^{w}, R^{x}, R^{y}, and R^{z} have the values described below.

R^{v} is hydrogen, halo, C₁₋₄ aliphatic, C₁₋₄ fluoroaliphatic, —OR⁵, —N(R⁴)₂, —CO₂R⁵, —C(O)N(R⁴)₂, -T³-OR⁵, -T³-N(R⁴)₂, -T³-CO₂R⁵, -T³-C(O)N(R⁴)₂, or an optionally substituted 5- or 6-membered aryl or heteroaryl. In some embodiments, R^{v} is hydrogen, an optionally substituted phenyl, pyridyl, or pyrimidinyl group, halo, C₁₋₄ aliphatic, C₁₋₄ fluoroaliphatic, —(CH₂)ₚ—OR^{1x}, —(CH₂)ₚ—N(R^{4x})(R^{4z}), —(CH₂)ₚ—CO₂R^{5x}, —(CH₂)ₚ—C(O)N(R^{4x})(R^{4z}), —(CH₂)_{q}—N(R^{4x})—(CH₂)_{q}—R^{5x}, —(CH₂)_{q}—N(R^{4x})—(CH₂)_{q}—R^{2x}, —(CH₂)_{q}—N(R^{4x})—(CH₂)S—R^{2y}—(CH₂)_{q}—N(R^{4x})C(═NR^{4x})—(CH₂)_{q}—R^{1x}, —(CH₂)_{q}—N(R^{4x})C(═NR^{4x})—(CH₂)_{q}—R^{2x}, or —(CH₂)_{q}—N(R^{4x})C(═NR^{4x})—(CH₂)_{q}—R^{2y}. In certain embodiments, R^{v} is hydrogen, halo, C₁₋₄ aliphatic, C₁₋₄ fluoroaliphatic, —(CH₂)ₚ—OR^{5x}, —(CH₂)ₚ—N(R^{4x})(R^{4z}), —(CH₂)ₚ—CO₂R^{5x}, —(CH₂)ₚ—C(O)N(R^{4x})(R^{4z}), or an optionally substituted phenyl, pyridyl, or pyrimidinyl group.

R^{w} is hydrogen, halo, C₁₋₄ aliphatic, C₁₋₄ fluoroaliphatic, —OR⁵, —N(R⁴)₂, —CO₂R⁵, —C(O)N(R⁴)₂.

Each R^{x} independently is hydrogen, fluoro, C₁₋₄ aliphatic, C₁₋₄ fluoroaliphatic, —CO₂R⁴, —C(O)N(R⁴)₂, -T³-N(R⁴)₂, -T³-OR⁵, -T³-CO₂R⁵, or -T³-C(O)N(R⁴)₂. In certain embodiments, each R^{x} independently is hydrogen, fluoro, C₁₋₄ aliphatic, C₁₋₄ fluoroaliphatic, —(CH₂)ₚ—CO₂R^{5x}, —(CH₂)ₚ—C(O)N(R^{4x})(R^{4z}), —(CH₂), —N(R^{4x})(R^{4z}), or —(CH₂)ᵣ—OR^{5x}.

R^{y} is hydrogen, halo, C₁₋₄ aliphatic, C₁₋₄ fluoroaliphatic, —OR⁵, —N(R⁴)₂, —CO₂R⁷, —C(O)N(R⁴)₂, -T³-OR⁵, -T³-N(R⁴)₂, -T³-CO₂R⁵, or -T³-C(O)N(R⁴)₂. In certain embodiments, R^{y} is hydrogen, fluoro, C₁₋₄ aliphatic, C₁₋₄ fluoroaliphatic, —(CH₂)ₚ—N(R^{4x})(R^{4z}), —(CH₂)ₚ—OR^{5x}, —(CH₂)ₚ—CO₂R^{5x}, —(CH₂)ₚ—C(O)N(R^{4x})(R^{4z}).

Each R^{z} independently is hydrogen, fluoro, C₁₋₄ aliphatic, or C₁₋₄ fluoroaliphatic.

T³ is a C₁₋₄ alkylene chain optionally substituted with one or two substituents independently selected from the group consisting of C₁₋₃ aliphatic, C₁₋₃ fluoroaliphatic, —F, —OH, —O(C₁₋₄ alkyl), —CO₂H, —CO₂(C₁₋₄ alkyl), —C(O)NH₂, and —C(O)NH(C₁₋₄ alkyl).

Each R^{1x} independently is an optionally substituted phenyl, piperidinyl, piperazinyl, morpholinyl, or pyrrolidinyl ring.

Each R^{2x} independently is —C(O)N(R^{4x})(R^{4z}).

Each R^{2y} independently is —N(R^{4x})(R^{4z}), —NR^{4x}C(O)R^{5x}, —N(R^{4x})—CO₂R^{2x}, —N(R^{4x})—C(═NR^{4x})—R^{5x} or —OR^{5x}.

Each R^{4x} independently is hydrogen, C₁₋₄ alkyl, C₁₋₄ fluoroalkyl, or C₆₋₁₀ ar(C₁₋₄)alkyl, the aryl portion of which may be optionally substituted, and each R⁴ independently is hydrogen, C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring; or R^{4x} and R^{4z}, taken together with the nitrogen atom to which they are attached, form an optionally substituted 4- to 8-membered heterocyclyl ring having, in addition to the nitrogen atom, 0-2 ring heteroatoms independently selected from N, O, and S.

Each R^{5x} independently is hydrogen, C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring.

The variable p is 0, 1, or 2; q, at each occurrence independently, is 1, 2, or 3, r is 1 or 2, and s is 2 or 3.

In more particular embodiments, Ring D is selected from the group consisting of:

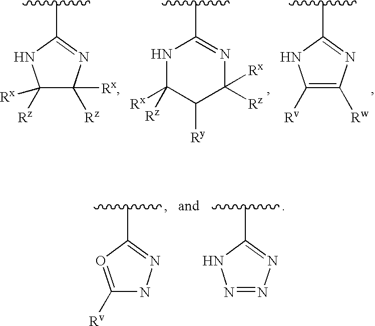

In still more particular embodiments, Ring D is selected from the group consisting of:

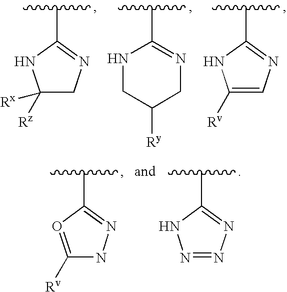

In certain particular embodiments, Ring B is selected from the group consisting of:

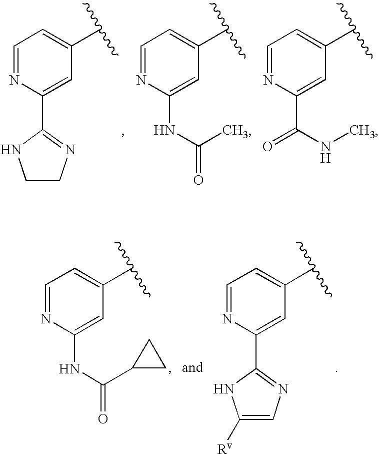

In the compounds of formulae (I)-(III), Ring C is an optionally substituted 5- or 6-membered aryl or heteroaryl ring having 0-3 ring nitrogen atoms and optionally one additional ring heteroatom selected from oxygen and sulfur. In some embodiments, two adjacent substituents on Ring C, taken together with the intervening ring atoms, form an optionally substituted fused Ring E. Ring E is a 5- or 6-membered aromatic or non-aromatic ring having 0-3 ring heteroatoms selected from the group consisting of O, N, and S.

In some embodiments, Ring C is an optionally substituted furanyl, thienyl, pyrrolyl, oxazolyl, thiazolyl, imidazolyl, pyrazolyl, isoxazolyl, isothiazolyl, oxadiazolyl, triazolyl, thiadiazolyl, phenyl, pyridyl, pyridazinyl, pyrimidinyl, pyrazinyl, or triazinyl, wherein one ring nitrogen atom in Ring C optionally is oxidized.

Each substitutable ring nitrogen atom in Ring C is unsubstituted or is substituted with —C(O)R⁵, —C(O)N(R⁴)₂, —CO₂R⁶, —SO₂R⁶, —SO₂N(R⁴)₂, or a C₁₋₄ aliphatic optionally substituted with —F, —OH, —O(C₁₋₄ alkyl), —CN, —N(R⁴)₂, —C(O)(C₁₋₄ alkyl), —CO₂H, —CO₂(C₁₋₄ alkyl), —C(O)NH₂, —C(O)NH(C₁₋₄ alkyl), or an optionally substituted C₆₋₁₀ aryl ring. One ring nitrogen atom in Ring C optionally is oxidized. In some embodiments, each substitutable ring nitrogen atom in Ring C is unsubstituted, and one ring nitrogen atom optionally is oxidized.

Substitutable ring carbon atoms in Ring C preferably are substituted with 0-2 R^{cc} and 0-2 R^{8c}. Each R^{8c} independently is selected from the group consisting of C₁₋₄ aliphatic, C₁₋₄ fluoroaliphatic, —O(C₁₋₄ alkyl), —O(C₁₋₄ fluoroalkyl), and halo. In some embodiments, R^{8c} is selected from the group consisting of halo, methyl, trifluoromethyl, ethyl, isopropyl, cyclopropyl, tert-butyl, methoxy, and trifluoromethoxy.

Each R^{cc} independently is halo, —NO₂, —CN, —C(R⁵)═C(R⁵)₂, —C≡C—R⁵—OR⁵, —SR⁶, —S(O)R⁶, —SO₂R⁶, —SO₂N(R⁴)₂—N(R⁴)₂, —NR⁴C(O)R⁵—NR⁴C(O)N(R⁴)₂—N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —C(═NR⁴)—N(R⁴)—OR⁷, —C(R⁶)═N—OR⁵, or an optionally substituted aliphatic, aryl, heteroaryl, or heterocyclyl; or two adjacent R^{cc}, taken together with the intervening ring atoms, form a fused Ring E.

In some embodiments, each R^{cc} independently is selected from the group consisting of C₁₋₆ aliphatic, C₁₋₆ fluoroaliphatic, halo, —R^{1c}, —R^{2c}, -T²-R^{2c}, and -T²-R^{1c}. The variables T², R^{1c}, and R^{2c} have the values described below.

T² is a C₁₋₆ alkylene chain optionally substituted with R^{3a} or R^{3b}, wherein the alkylene chain optionally is interrupted by —C(R⁵)═C(R⁵)—, —C≡C—, —O—, —S—, —S(O)—, —S(O)₂—, —SO₂N(R⁴)—, —N(R⁴)—, —N(R⁴)C(O)—, —NR⁴C(O)N(R⁴)—, —N(R⁴)CO₂—, —N(R⁴)SO₂—, —C(O)N(R⁴)—, —C(O)—, —CO₂—, —OC(O)—, or —OC(O)N(R⁴)—, and wherein T² or a portion thereof optionally forms part of a 3-7 membered ring. In some embodiments, T² is a C₁₋₄ or C₂₋₄ alkylene chain optionally substituted with R^{3a} or R^{3b}. In some embodiments, T² is a C₁₋₄ alkylene chain optionally substituted with one or two groups independently selected from —F, C₁₋₄ aliphatic, and C₁₋₄ fluoroaliphatic.

Each R^{1c} independently is an optionally substituted aryl, heteroaryl, heterocyclyl, or cycloaliphatic ring.

Each R^{2c} independently is —NO₂, —CN, —C(R⁵)═C(R⁵)₂, —C≡C—R⁴, —OR⁵, —SR⁶, —S(O)R⁶, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —C(═NR⁴)—N(R⁴)—OR⁵, or —C(R⁶)═N—OR⁵. In some embodiments, each R^{2c} independently is —CN, —C(R⁵)═C(R⁵)₂, —C≡C—R⁵, —OR⁵, —SR⁶, —N(R⁴)₂, —NR⁴C(O)R⁵, —NR⁴C(O)N(R⁴)₂, —NR⁴CO₂R⁶, —CO₂R⁵, or —C(O)N(R⁴)₂.

The variables R^{3a}, R^{3b}, R⁴, R⁵, R⁶, and R⁷ have the values described above for Ring B.

In some embodiments, the substitutable ring carbon atoms in Ring C are substituted with 0-2 R^{cc} and 0-1 R^{8c}, where:
each R^{cc} preferably is selected from the group consisting of C₁₋₄ aliphatic, C₁₋₄ fluoroaliphatic, halo, —R^{2c} and -T²-R^{2c}; or two adjacent R^{cc}, taken together with the intervening ring atoms, form a fused Ring E;
T² is a C₁₋₄alkylene chain optionally substituted with one or two groups independently selected from —F, C₁₋₄ aliphatic, and C₁₋₄ fluoroaliphatic;
each R^{2c} independently is —CN, —C(R⁵)═C(R⁵)₂, —C≡C—R⁵, —OR⁵, —SR⁶, —N(R⁴)₂, —NR⁴C(O)R⁵, —NR⁴C(O)N(R⁴)₂, —NR⁴CO₂R⁵, —CO₂R⁴, and —C(O)N(R⁴)₂; and
each R^{8c} independently is selected from the group consisting of C₁₋₄ aliphatic, C₁₋₄ fluoroaliphatic, —O(C₁₋₄ alkyl), —O(C₁₋₄ fluoroaliphatic), and halo.

In some embodiments, the substitutable ring carbon atoms in Ring C are substituted with 0-2 R^{cc} and 0-1 R^{8c}, where:
each R^{cc} independently is halo, —CN, —C(R^{5x})═C(R^{5x})(R^{5y}), —C≡C—R^{5y}, —OR^{5y}, —SR^{6x}, —N(R^{4x})(R^{4y}), —CO₂R^{5x}, —C(O)N(R^{4x})(R^{4y}), or a C₁₋₄ aliphatic or C₁₋₄ fluoroaliphatic optionally substituted with one or two substituents independently selected from the group consisting of —OR^{5x}, —N(R^{4x})(R^{4y}), —SR^{6x}, —CO₂R^{5x}, or —C(O)N(R^{4x})(R^{4y}); or two adjacent R^{cc}, taken together with the intervening ring atoms, form a fused Ring E;
R^{4x} is hydrogen, C₁₋₄ alkyl, C₁₋₄ fluoroalkyl, or C₆₋₁₀ ar(C₁₋₄)alkyl, the aryl portion of which may be optionally substituted, or two R^{4x} on the same nitrogen atom, taken together with the nitrogen atom, form an optionally substituted 4- to 8-membered heterocyclyl ring having, in addition to the nitrogen atom, 0-2 ring heteroatoms independently selected from N, O, and S;
R^{4y} is hydrogen, C₆₋₁₀ ar(C₁₋₄)alkyl, the aryl portion of which may be optionally substituted, an optionally substituted 5- or 6-membered aryl, heteroaryl, or heterocyclyl ring, or a C₁₋₄ alkyl or C₁₋₄-fluoroalkyl optionally substituted with one or two substituents independently selected from the group consisting of —OR^{5x}, —N(R^{4x})₂, —CO₂R^{5x}, or —C(O)N(R^{4x})₂; or
R^{4x} and R^{4y}, taken together with the nitrogen atom to which they are attached, form an optionally substituted 4- to 8-membered heterocyclyl ring having, in addition to the nitrogen atom, 0-2 ring heteroatoms independently selected from N, O, and S;
each R^{5x} independently is hydrogen, C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring;
each R^{5y} independently is hydrogen, an optionally substituted C₆₋₁₀ aryl, a C₆₋₁₀ar(C₁₋₄)alkyl, the aryl portion of which may be optionally substituted, or a C₁₋₄ alkyl or C₁₋₄ fluoroalkyl optionally substituted with one or two substituents independently selected from the group consisting of —OR^{5x}, —N(R^{4x})₂, —CO₂R^{4x}, or —C(O)N(R^{4x})₂; and
each R^{6x} independently is C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring.

When two adjacent R^{cc}, taken together with the intervening ring atoms, form a fused Ring E, each substitutable saturated ring carbon atom in Ring E is unsubstituted or is substituted with ═O, ═S, ═C(R⁵)₂, or —R^{ee}. Each substitutable unsaturated ring carbon atom in Ring E is unsubstituted or is substituted with —R^{ee}. Each substitutable ring nitrogen atom in Ring E is unsubstituted or is substituted with —C(O)R⁵, —C(O)N(R⁴)₂, —CO₂R⁶, —SO₂R⁶, —SO₂N(R⁴)₂, C₁₋₄ aliphatic, an optionally substituted C₆₋₁₀ aryl, or a C₆₋₁₀ ar(C₁₋₄)alkyl, the aryl portion of which is optionally substituted. One ring nitrogen or sulfur atom in Ring E optionally is oxidized.

Each R^{ee} independently is halo, —NO₂, —CN, —C(R⁵)═C(R⁵)₂, —C≡C—R⁵, —OR⁵, —SR⁶, —S(O)R⁶, —SO₂R⁶, —SO₂ N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —C(═NR⁴)—N(R⁴)—OR¹, —C(R⁶)═N—OR⁵, or an optionally substituted C₁₋₆ aliphatic.

In some embodiments, each R^{ee} independently is selected from the group consisting of C₁₋₆ aliphatic, C₁₋₆ fluoroaliphatic, halo, —R^{2e}, -T⁴-R^{2e}, and -T⁴-R^{1e};
T⁴ is a C₁₋₆ alkylene chain optionally substituted with R^{3a} or R^{3b};
each R^{1e} independently is an optionally substituted aryl, heteroaryl, heterocyclyl, or cycloaliphatic ring; and
each R^{1e} independently is —NO₂, —CN, —C(R⁷)═C(R⁵)₂, —C≡C—R⁵, —OR⁵, —SR⁶, —S(O)R⁶, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴), —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁷, —C(═NR⁴)—N(R⁴)—OR⁷, or —C(R⁶)═N—OR⁵.

The variables R^{3a}, R^{3b}, R⁴, R⁵, R⁶, and R⁷ have the values described above for Ring B.

In some embodiments, each R^{ee} is selected from the group consisting of C₁₋₄ aliphatic, C₁₋₄ fluoroaliphatic, halo, —R^{2e}, and -T⁴-R^{2e};
T⁴ is a C₁₋₄ alkylene chain optionally substituted with one or two groups independently selected from —F, C₁₋₄ aliphatic, and C₁₋₄ fluoroaliphatic; and
each R^{2e} independently is —CN, —C(R⁵)═C(R⁵)₂, —C≡C—R⁵, —OR⁵, —SR⁶, —N(R⁴)₂, —NR⁴C(O)R⁵, —NR⁴C(O)N(R⁴)₂, —NR⁴CO₂R⁶, —CO₂R⁵, or —C(O)N(R⁴)₂.

In some embodiments, Ring C is a 5- or 6-membered heteroaryl substituted with 0-2 R^{cc}. In some such embodiments, each R^{cc} independently is selected from the group consisting of -halo, C₁₋₄ alkyl, C₁₋₄ fluoroalkyl, —O(C₁₋₄ alkyl), and —O(C₁₋₄ fluoroalkyl), or two adjacent R^{cc}, taken together with the intervening ring atoms, form a fused Ring E, where Ring E is a 5- or 6-membered aromatic or non-aromatic ring having 0-3 ring heteroatoms selected from the group consisting of O, N, and S. In certain such embodiments, Ring E is an optionally substituted benzo ring.

In certain particular embodiments, Ring C is selected from the group consisting of:

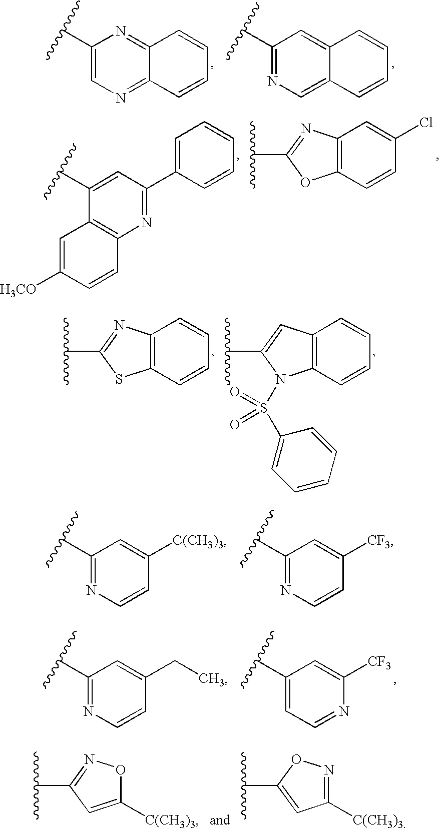

In some other embodiments, Ring C is an optionally substituted phenyl. In some such embodiments, Ring C is selected from the group consisting of: 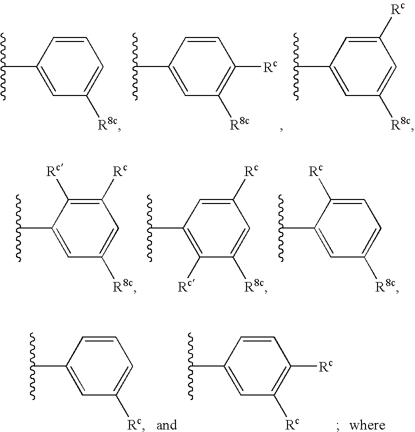
each R^{cc} independently is halo, —CN, —C(R^{5x})═C(R^{5x})(R^{5y}), —C≡C—R^{5y}, —OR^{5y}, —SR^{6x}, —N(R^{dx})(R^{dy}), —CO₂R^{5x}, —C(O)N(R^{4x})(R^{4y}), or a Cl₄ aliphatic or C₁₋₄ fluoroaliphatic optionally substituted with one or two substituents independently selected from the group consisting of —OR^{5x}, —N(R^{4x})(R^{4y}), —SR^{6x}, —CO₂R^{5x}, or —C(O)N(R^{4x})(R^{4y}); or two adjacent R^{cc}, taken together with the intervening ring atoms, form a fused Ring E, where Ring E is a 5- or 6-membered aromatic or non-aromatic ring having 0-3 ring heteroatoms selected from the group consisting of O, N, and S;
R^{c′} is C₁₋₄ aliphatic, C₁₋₄ fluoroaliphatic, halo, —CN, —OH, —O(C₁₋₄ alkyl), —O(C₁₋₄ fluoroalkyl), —S(C₁₋₄ alkyl), —NH₂, —NH(C_{—}4 alkyl), or —N(C₁₋₄ alkyl)₂;
R^{8c} is C₁₋₄ aliphatic, C₁₋₄ fluoroaliphatic, or halo; and
the variables R^{4x}, R^{4y}, R^{5x}, R^{5y}, and R^{6x} have the values described above for formula (I).

In certain particular embodiments, Ring C is selected from the group consisting of:

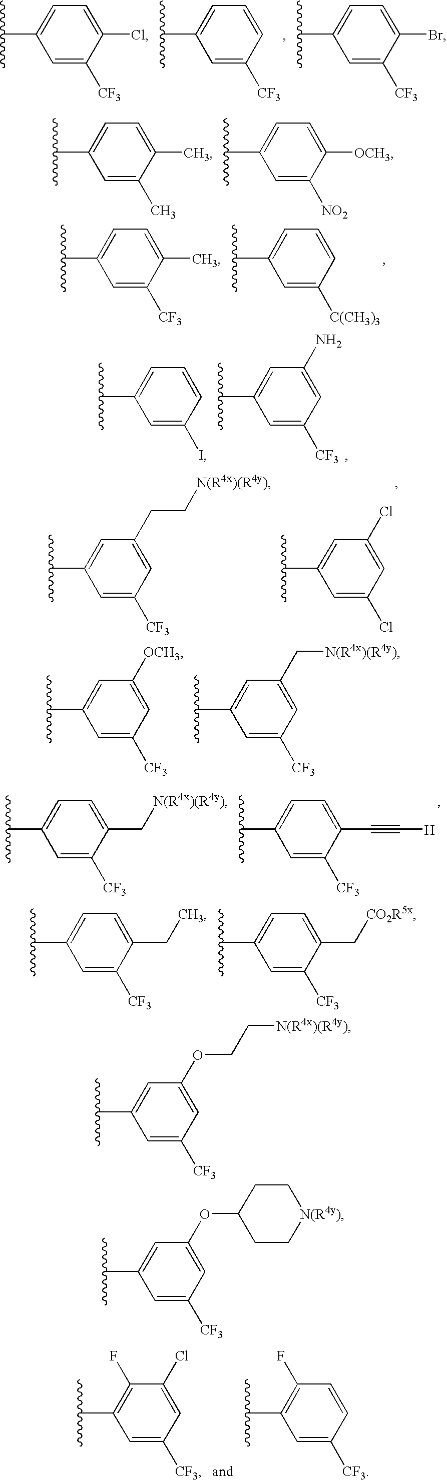

In certain other embodiments, Ring C is selected from the group consisting of:

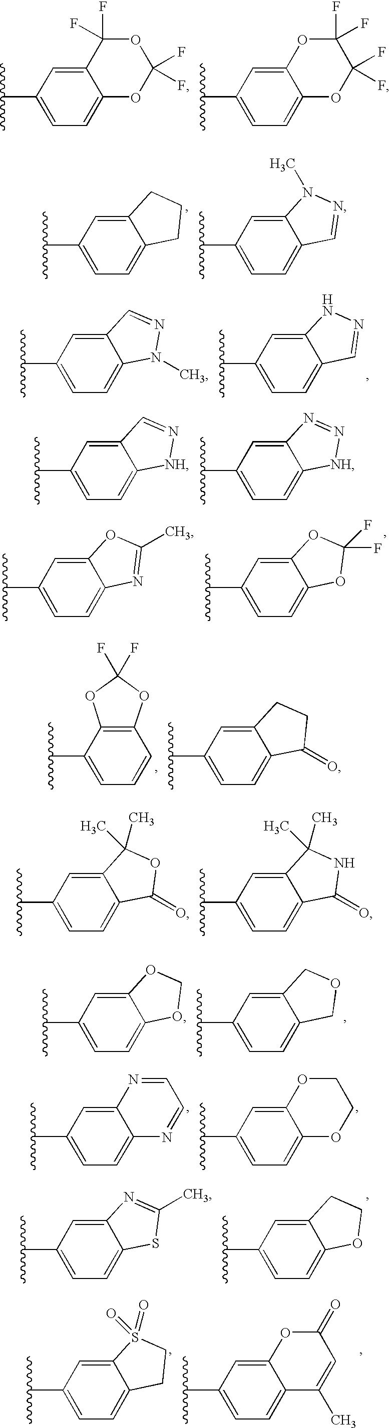

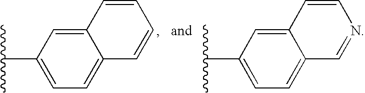

The invention also relates to a subgenus of the compounds of formula (I), characterized by formula (IV): 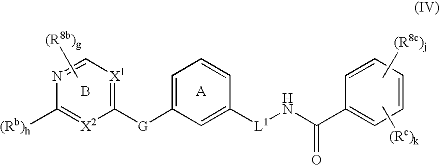
or a pharmaceutically acceptable salt thereof;
wherein:
G is —O— or —NH—;
X¹ and X² are each independently CH or N, provided that X¹ and X² are not both N;
one ring nitrogen atom in Ring B optionally is oxidized;
g is 0 or 1;
h is 0 or 1;
j is 0 or 1;
k is 0, 1, or 2; and
Ring A and the variables R^{bb}, R^{8b}, R^{cc}, and R^{8c} have the values and preferred values described above for formulae (I)-(III).

In some embodiments, the invention relates to a compound of formula (IV), wherein:
X¹ and X² are each CH;
Ring A is substituted with zero occurrences of R^{aa};
each R^{cc} independently is halo, —CN, —C(R^{5x})═C(R^{5x})(R^{5y}), —C≡C—R^{5y}, —OR^{1e}, —SR^{6x}, —CO₂R^{5x}, —C(O)N(R^{4x})(R^{4y}), or a C₁₋₄ aliphatic or C₁₋₄ fluoroaliphatic optionally substituted with one or two substituents independently selected from the group consisting of —OR^{5x}, —N(R^{4x})(R^{4y}), —SR^{6x}, —CO₂R^{5x}, or —C(O)N(R^{4x})(R^{4y}); or two adjacent R^{cc}, taken together with the intervening ring atoms, form an optionally substituted fused 5- or 6-membered aromatic or non-aromatic ring having 0-3 ring heteroatoms independently selected from the group consisting of O, N, and S;
   R^{4x} is hydrogen, C₁₋₄ alkyl, C₁₋₄ fluoroalkyl, or C₆₋₁₀ ar(C₁₋₄)alkyl, the aryl portion of which may be optionally substituted, or two R^{4x} on the same nitrogen atom, taken together with the nitrogen atom, form an optionally substituted 4- to 8-membered heterocyclyl ring having, in addition to the nitrogen atom, 0-2 ring heteroatoms independently selected from N, O, and S;
   R^{4y} is hydrogen, C₆₋₁₀ ar(C₁₋₄)alkyl, the aryl portion of which may be optionally substituted, an optionally substituted 5- or 6-membered aryl, heteroaryl, or heterocyclyl ring, or a C₁₋₄ alkyl or C₁₋₄ fluoroalkyl optionally substituted with one or two substituents independently selected from the group consisting of —OR^{5x}, —N(R^{4x})₂, —CO₂R^{5x}, or —C(O)N(R^{4x})₂; or
   R^{4x} and R^{4y}, taken together with the nitrogen atom to which they are attached, form an optionally substituted 4- to 8-membered heterocyclyl ring having, in addition to the nitrogen atom, 0-2 ring heteroatoms independently selected from N, O, and S;
   each R^{5x} independently is hydrogen, C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring;
   each R^{5y} independently is hydrogen, an optionally substituted C₆₋₁₀ aryl, a C₆₋₁₀ ar(C₁₋₄)alkyl, the aryl portion of which may be optionally substituted, or a C₁₋₄ alkyl or C₁₋₄ fluoroalkyl optionally substituted with one or two substituents independently selected from the group consisting of —OR^{5x}, —N(R^{4x})₂—CO₂R^{5x}, or —C(O)N(R^{4x})₂; and
   each R^{6x} independently is C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring.

The invention also relates to a compound of formula (V): 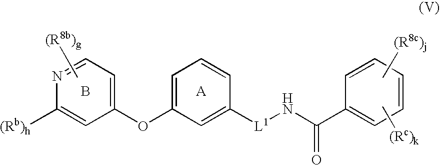
or a pharmaceutically acceptable salt thereof;
wherein:
g is 0 or 1;
j is 0 or 1;
k is 0, 1, or 2; and
the variables L¹, R^{bb}, R^{8b}, R^{cc}, and R^{8c} have the values and preferred values described above for formulae (I)-(IV).

In a preferred embodiment the compound of formula (I) is other than 6-[4-(2-benzoylamino-ethyl)-phenoxy]-nicotinamide.

Specific examples of compounds of formula (I) are shown below in Table 1.

TABLE 1
Raf Kinase Inhibitors

| | |
|---|---|
| 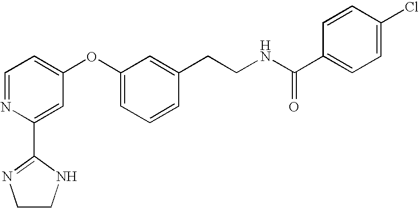 | 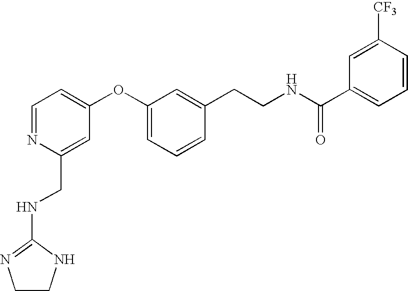 |
| I-1 | I-2 |

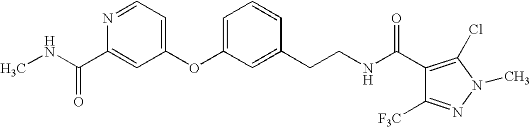

I-3

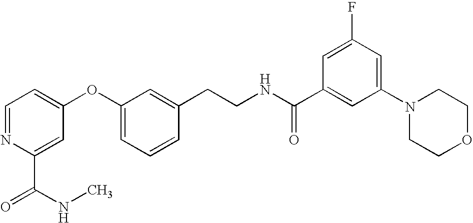

I-4

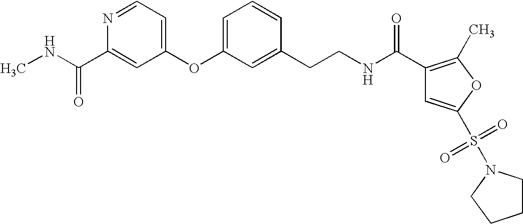

I-5

| | |
|---|---|
| 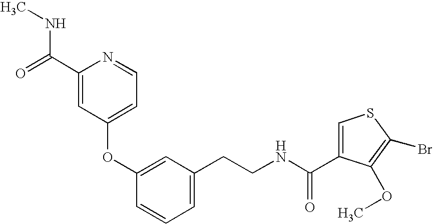 | 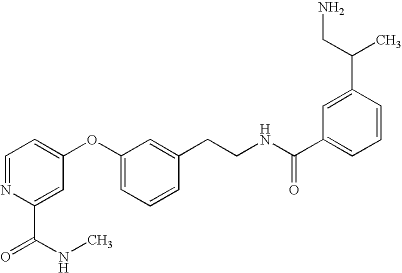 |
| I-6 | I-7 |

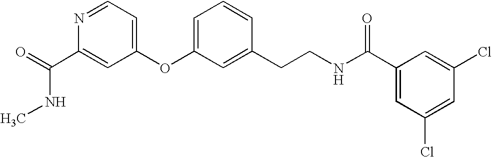

I-8

| | |
|---|---|
| 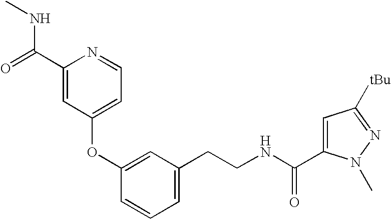 | 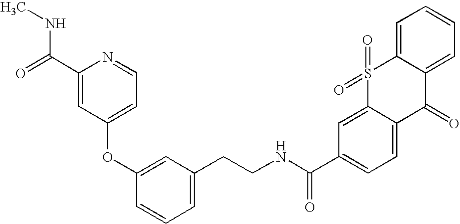 |
| I-9 | I-10 |

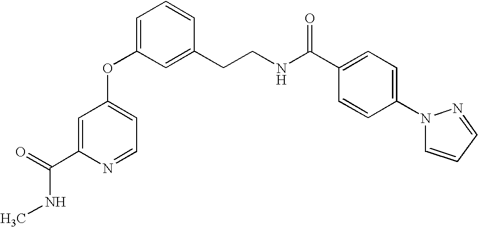

I-11

| | |
|---|---|
| 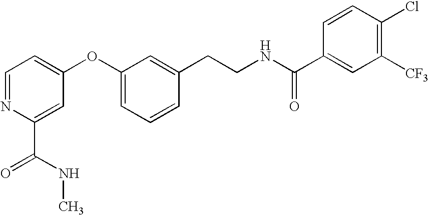 | 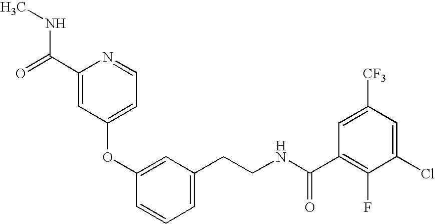 |
| I-12 | I-13 |

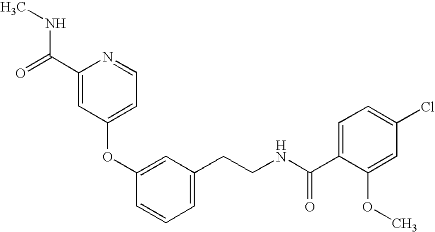

I-14

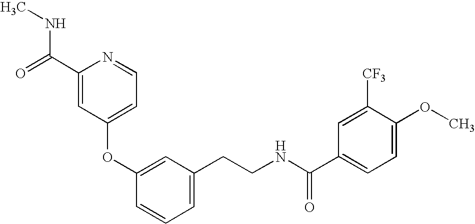

I-15

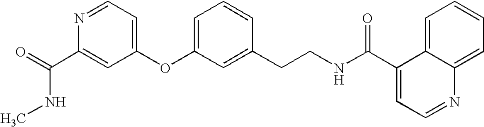

I-16

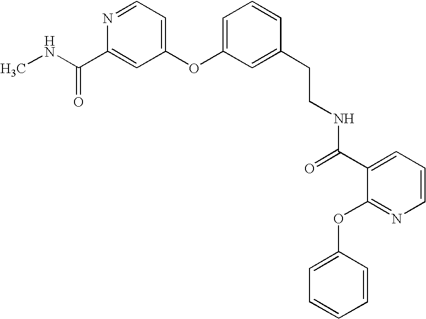

I-17

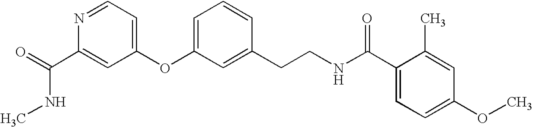

I-18

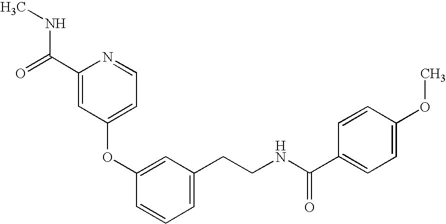

I-19

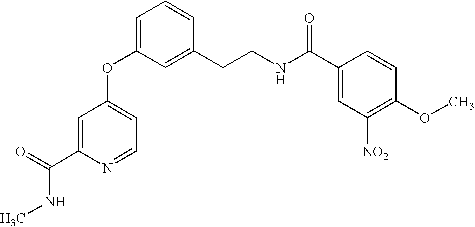

I-20

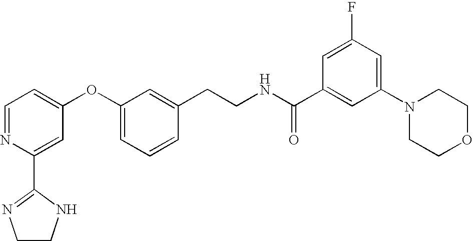

I-21

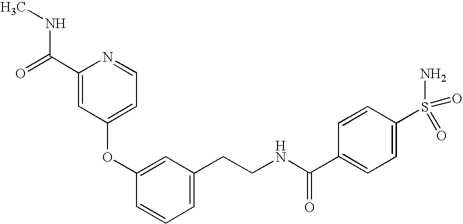

I-22

| | |
|---|---|
| 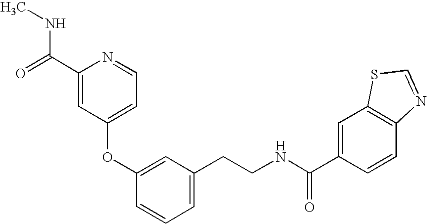 | 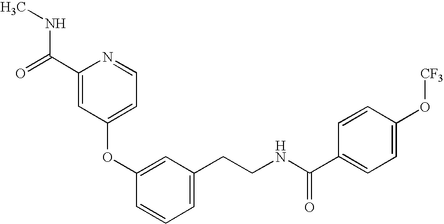 |
| I-23 | I-24 |

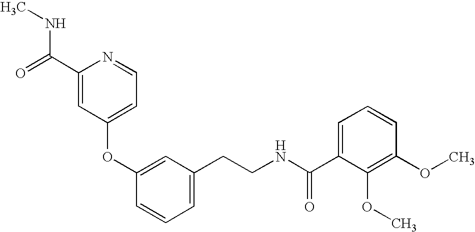

I-25

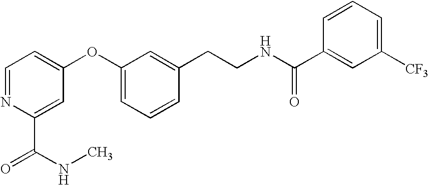

I-26

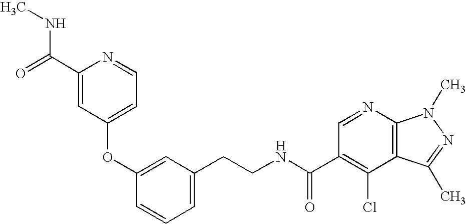

I-27

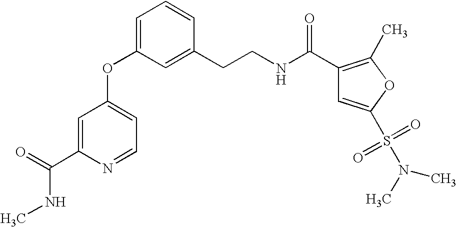

I-28

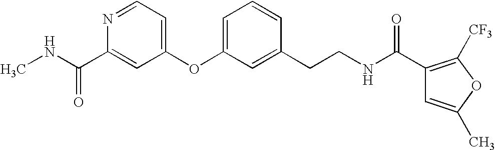

I-29

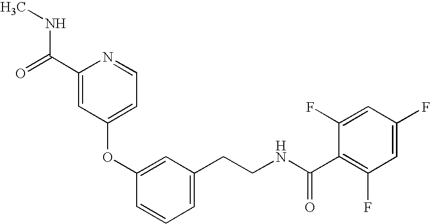

I-30

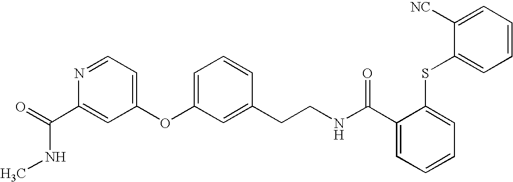

I-31

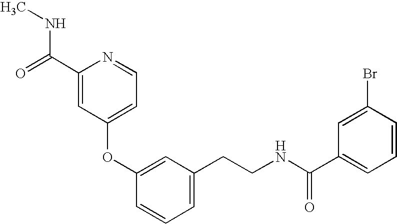

I-32

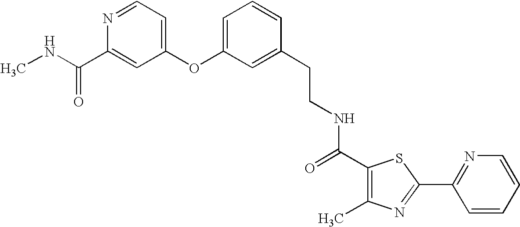

I-33

| | |
|---|---|
| 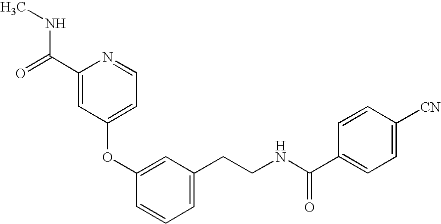 | 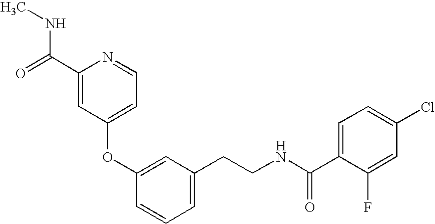 |
| I-34 | I-35 |

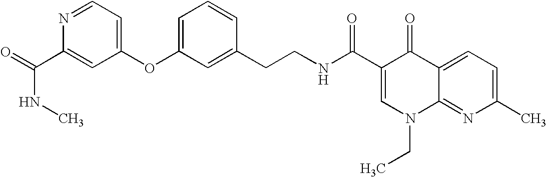

I-36

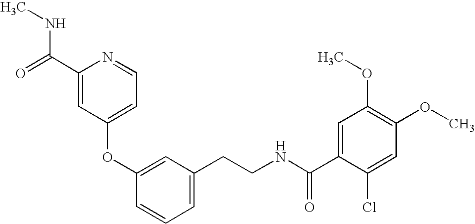

I-37

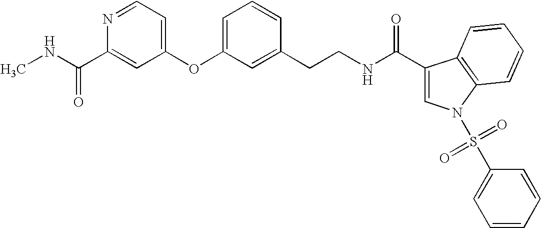

I-38

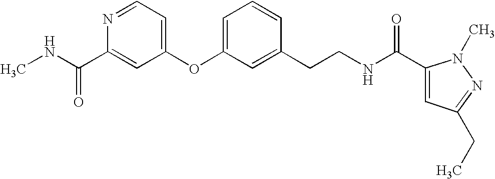

I-39

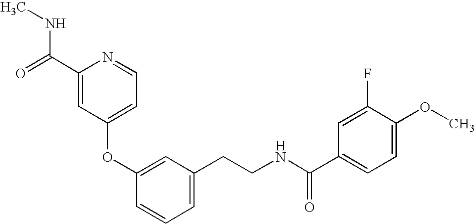

I-40

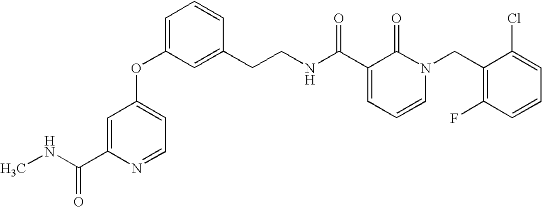

I-41

| | |
|---|---|
| 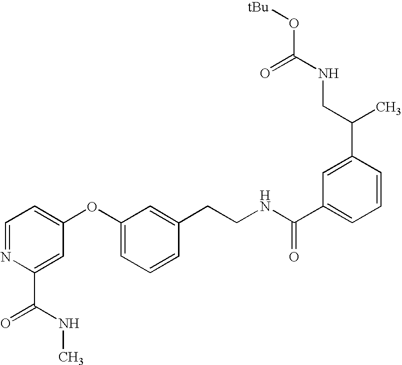 | 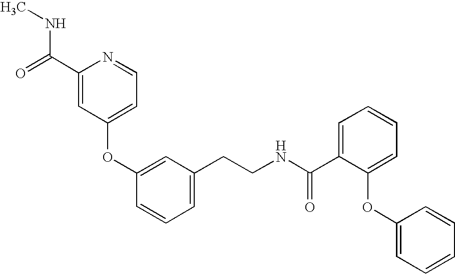 |
| I-42 | I-43 |

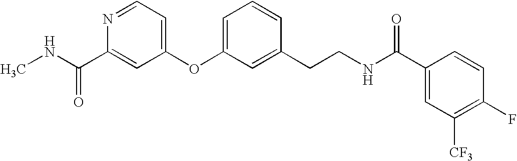

I-44

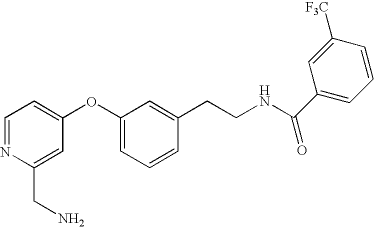

I-45

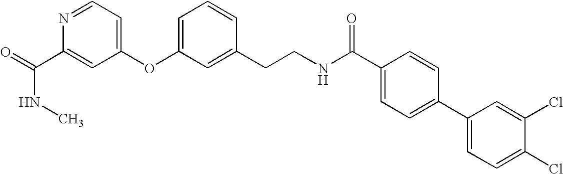

I-46

| | |
|---|---|
| 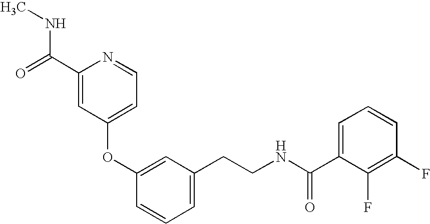 | 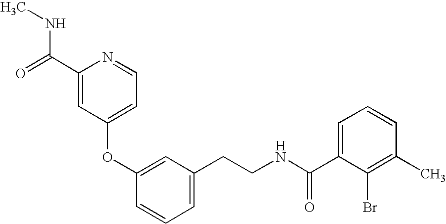 |
| I-47 | I-49 |
| 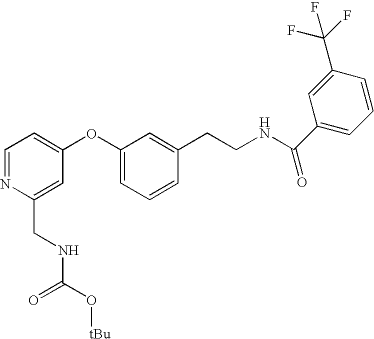 | 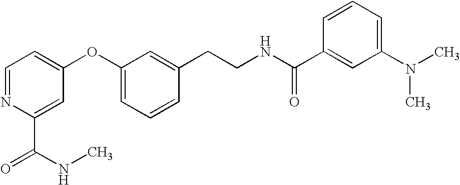 |
| I-50 | I-51 |
| 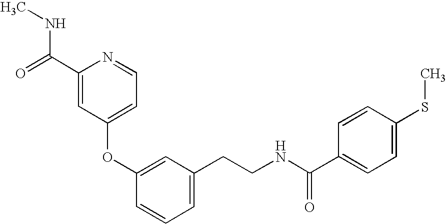 | 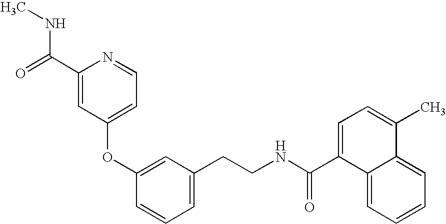 |
| I-52 | I-53 |

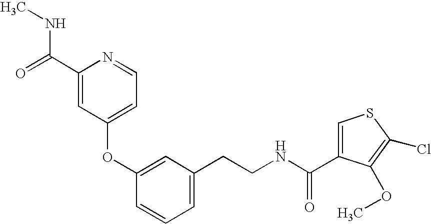

I-54

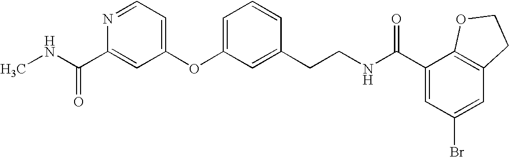

I-55

| | |
|---|---|
| 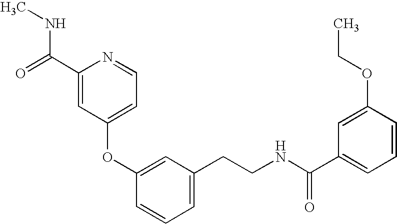 | 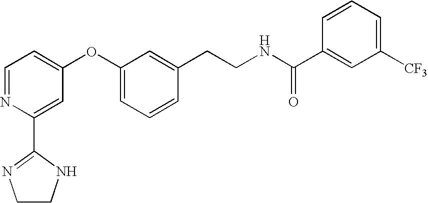 |
| I-56 | I-57 |

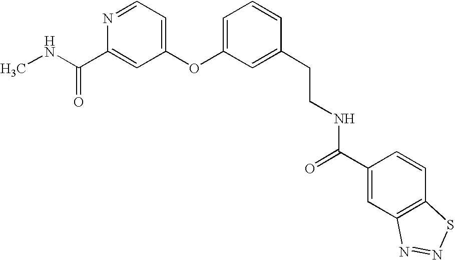

I-58

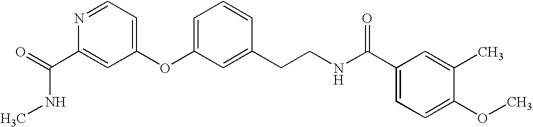

I-59

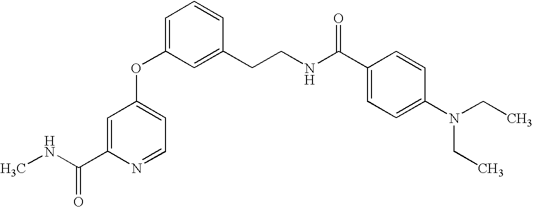

I-60

| | |
|---|---|
| 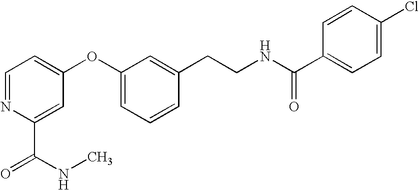 | 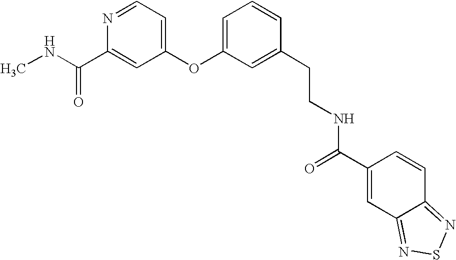 |
| I-61 | I-62 |
| 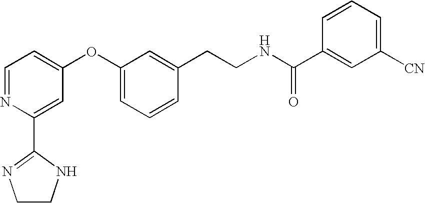 | 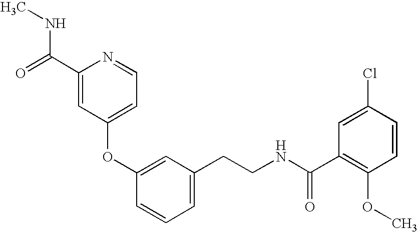 |
| I-63 | I-64 |

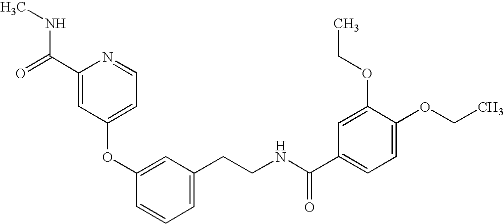

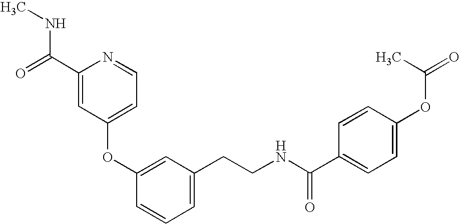

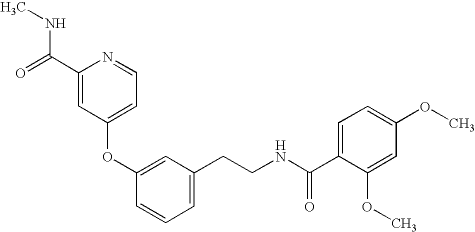

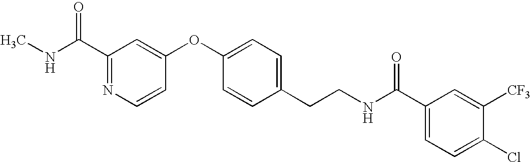

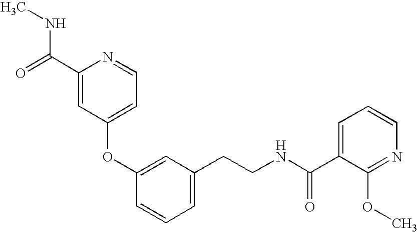

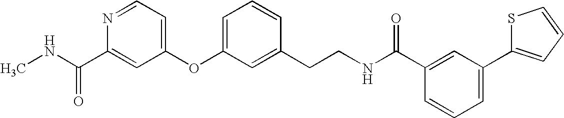

| | |
|---|---|
| 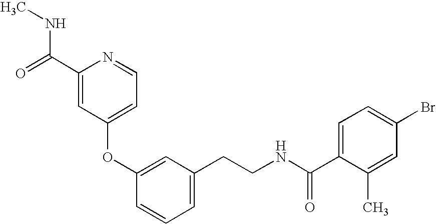 | 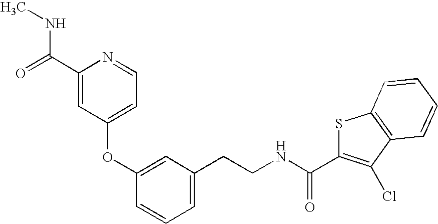 |
| I-71 | I-72 |

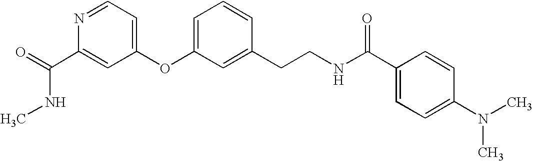

I-73

| | |
|---|---|
| 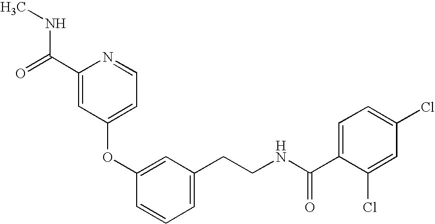 | 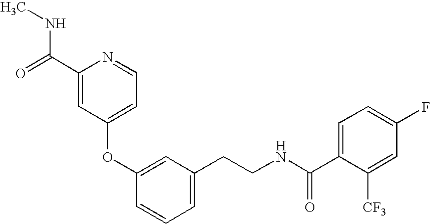 |
| I-74 | I-75 |

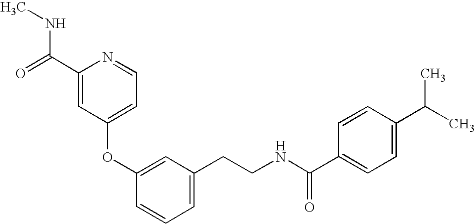

I-76

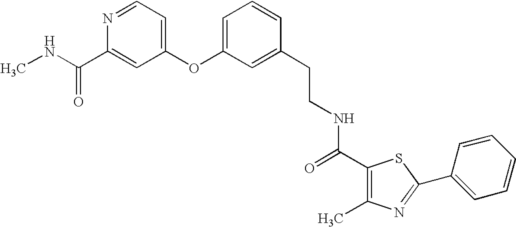

I-77

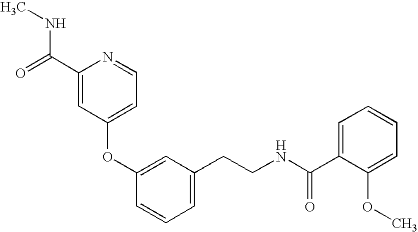

I-78

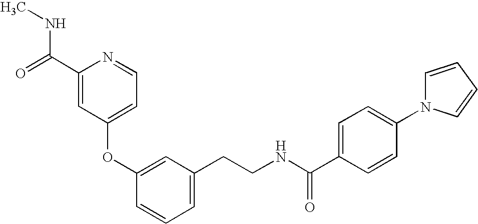

I-79

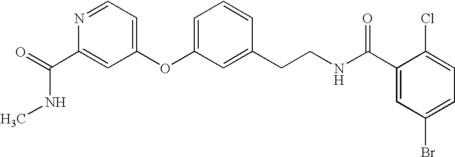

I-81

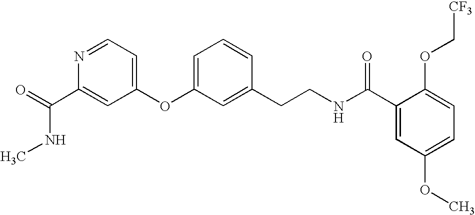

I-82

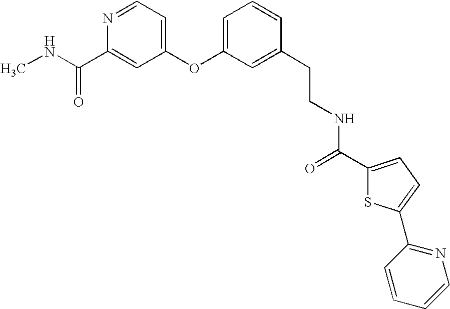

I-83

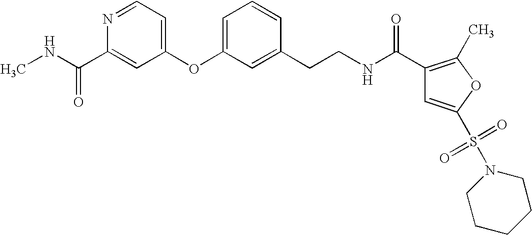

I-84

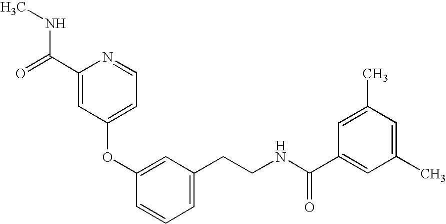

I-85

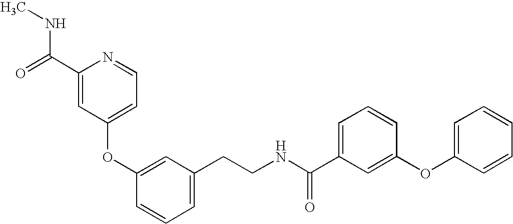

I-86

| | |
|---|---|
| 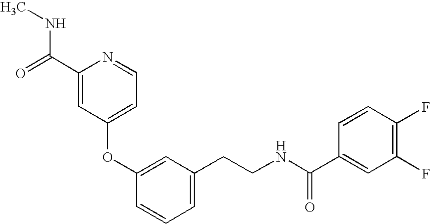 | 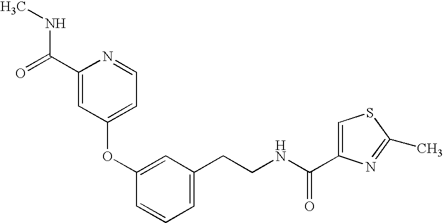 |
| I-87 | I-88 |
| 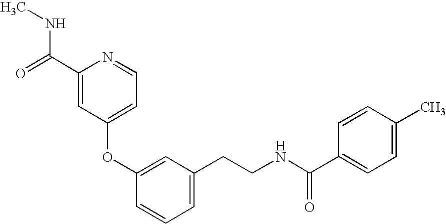 | 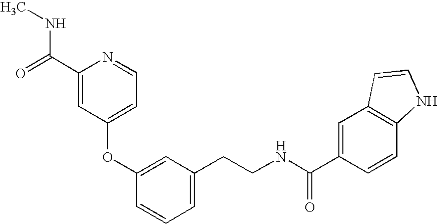 |
| I-89 | I-91 |
| 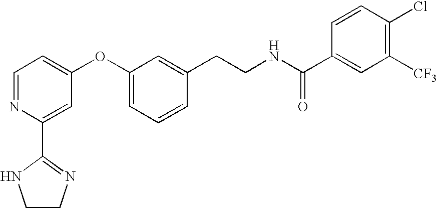 | 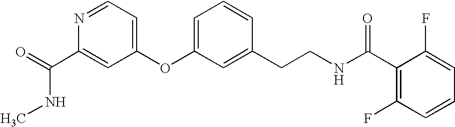 |
| I-92 | I-93 |
| 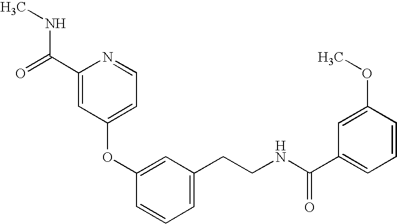 | 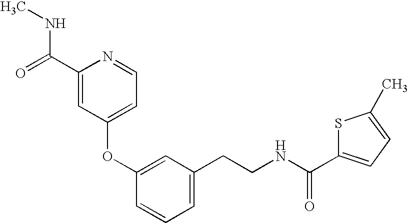 |
| I-94 | I-95 |

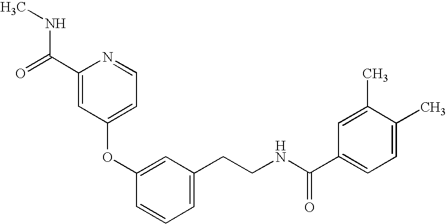

I-96

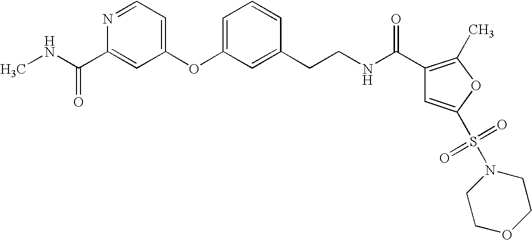

I-98

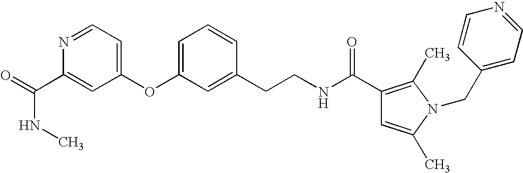

I-99

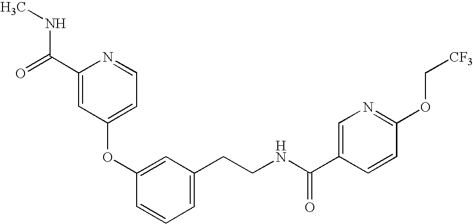

I-100

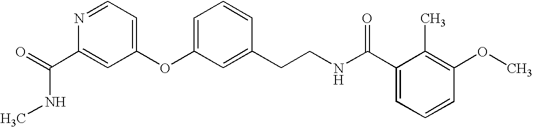

I-101

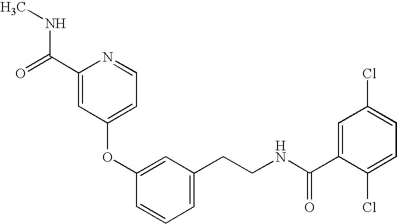

I-102

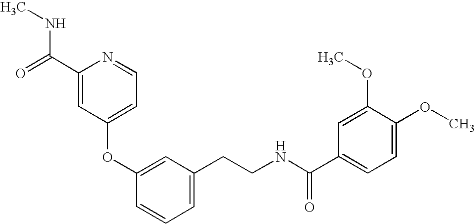

I-103

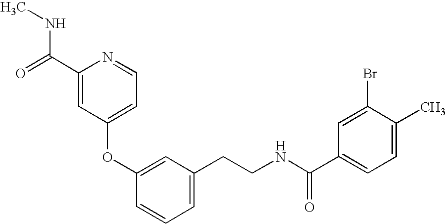

I-104

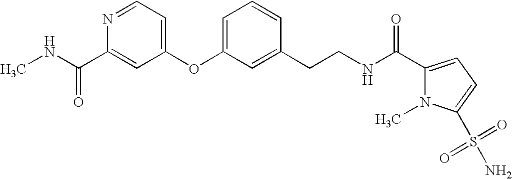

I-105

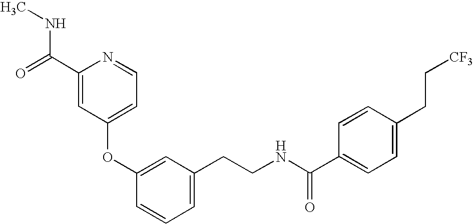

I-106

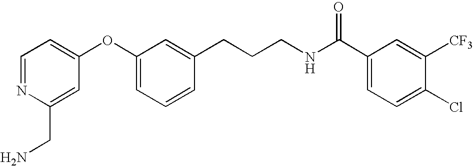

I-107

| | |
|---|---|
| 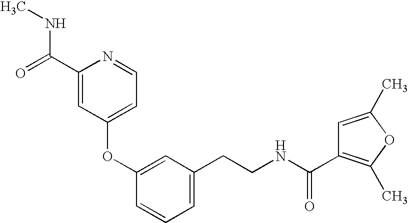 | 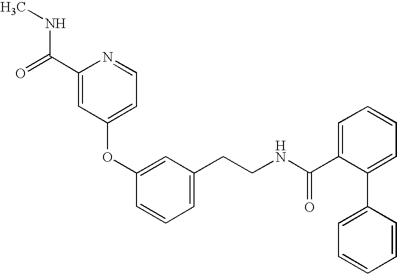 |
| I-108 | I-109 |

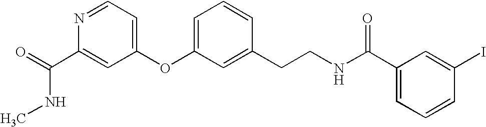

I-110

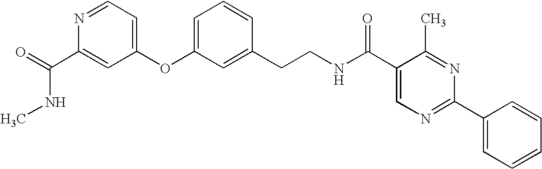

I-111

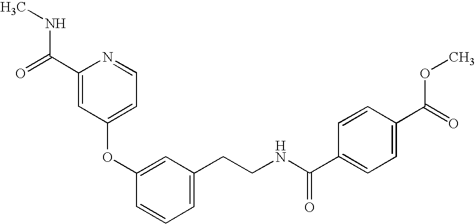

I-112

| | |
|---|---|
| 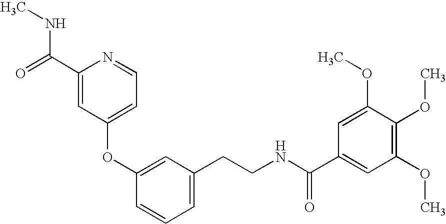 | 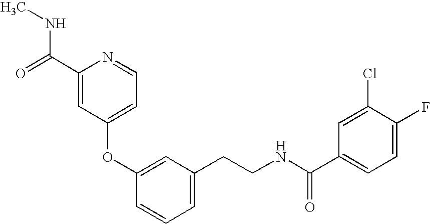 |
| I-114 | I-115 |

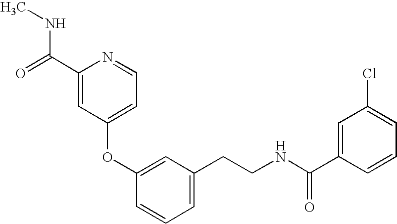

I-116

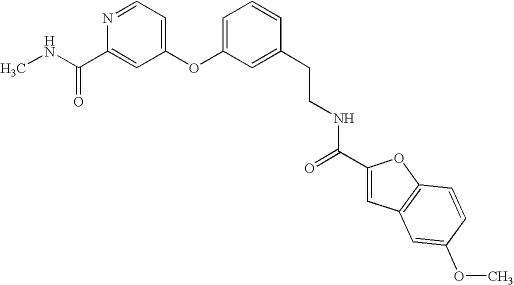

I-117

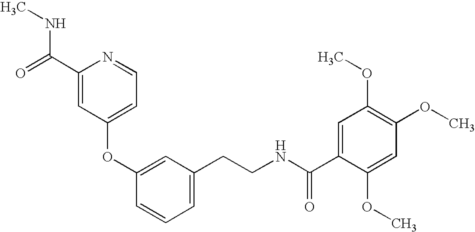

I-118

| | |
|---|---|
| 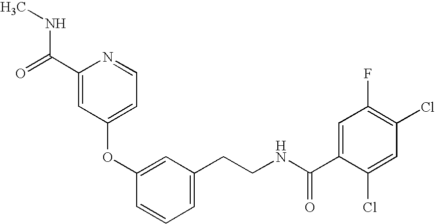 | 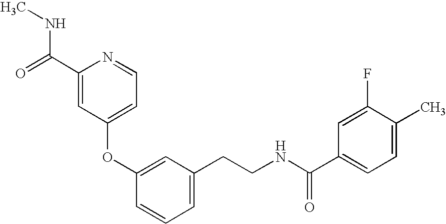 |
| I-119 | 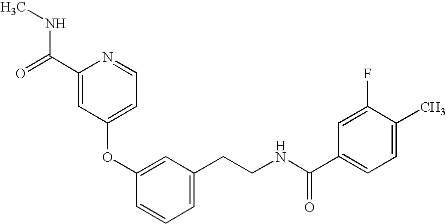 |

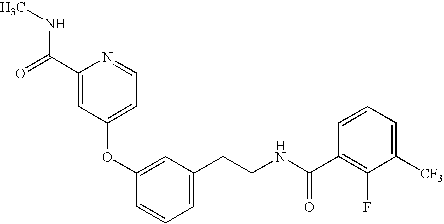

I-121

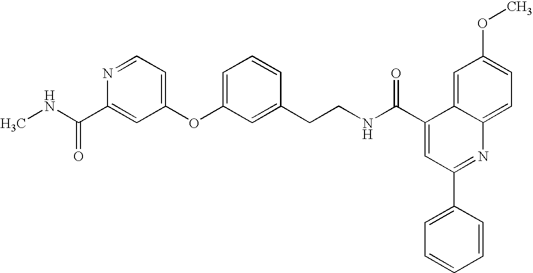

I-122

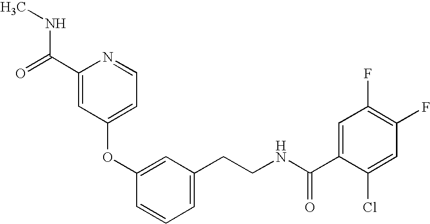

I-124

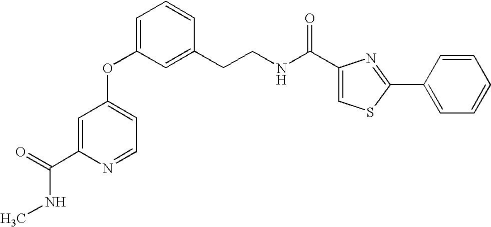

I-125

| | |
|---|---|
| 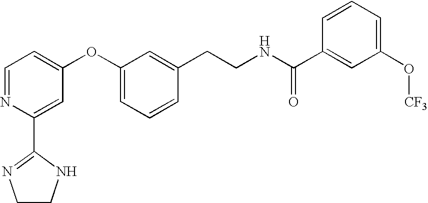 | 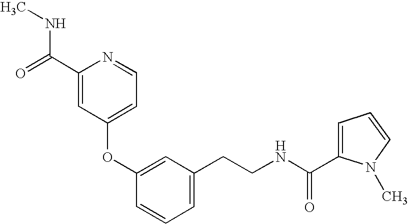 |
| I-126 | I-127 |

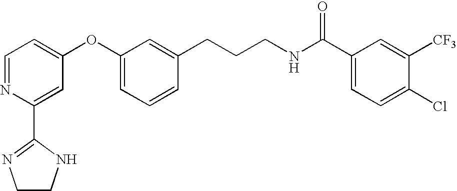

I-128

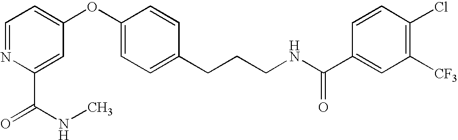

I-129

| | |
|---|---|
| 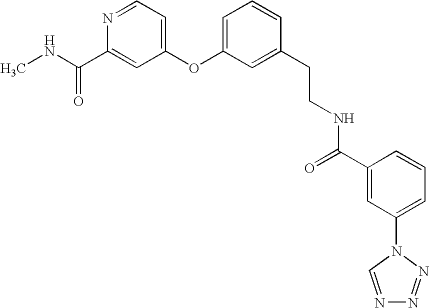 | 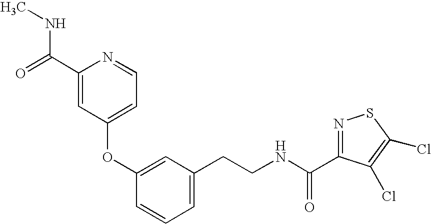 |
| I-130 | I-131 |
| 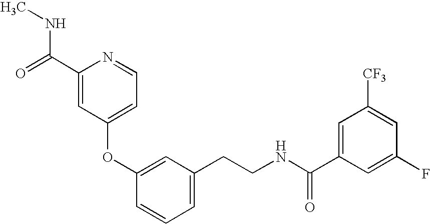 | 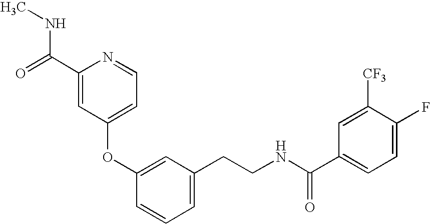 |
| I-132 | I-133 |

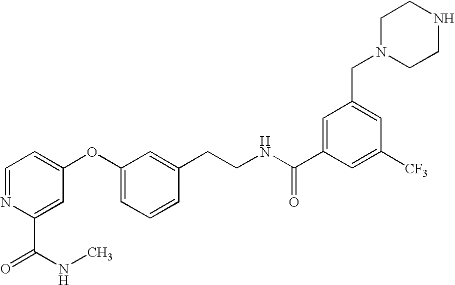

I-134

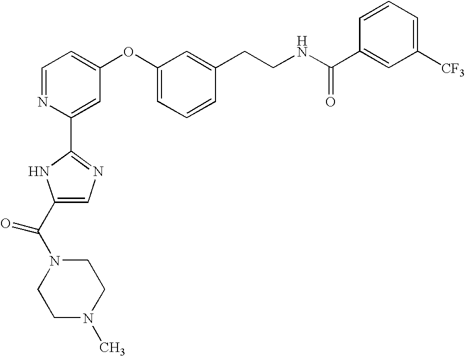

I-135

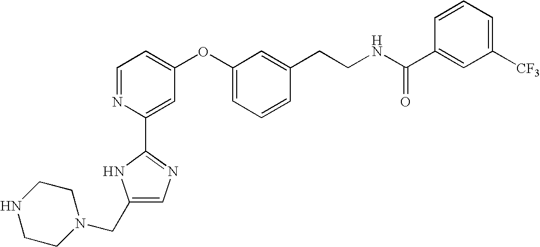

I-136

| | |
|---|---|
| 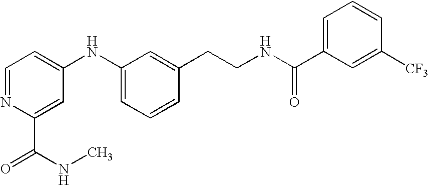 | 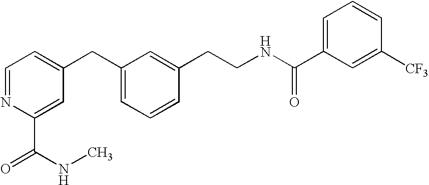 |
| I-137 | I-138 |
| 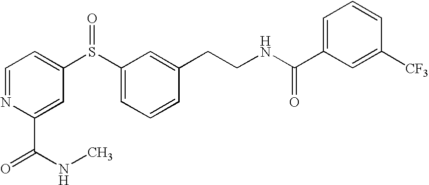 | 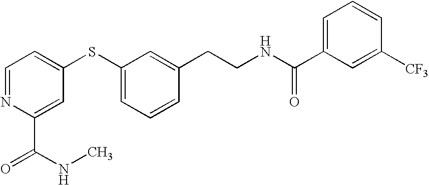 |
| I-139 | I-140 |
| 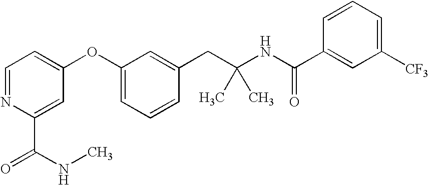 | 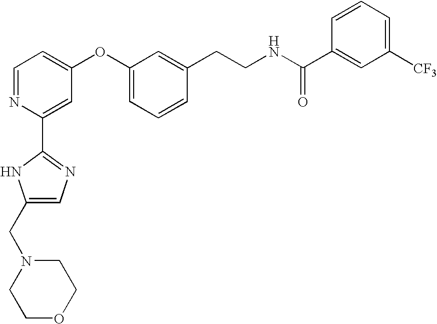 |
| I-141 | I-142 |
| 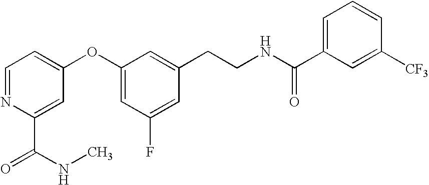 | 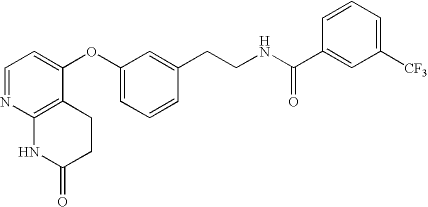 |
| I-143 | I-144 |
| 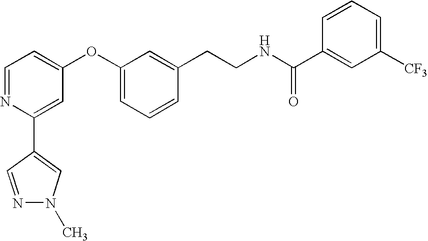 | 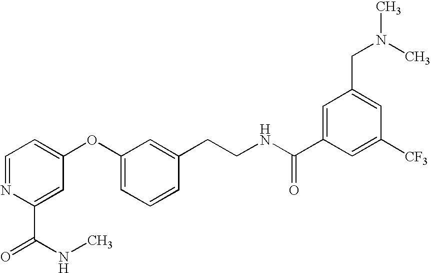 |
| I-145 | I-146 |
| 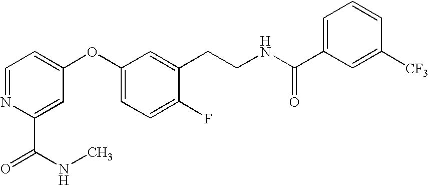 | 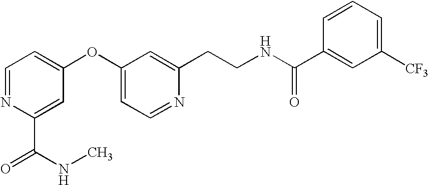 |
| I-147 | I-148 |

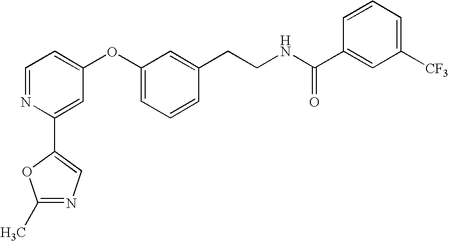

I-149

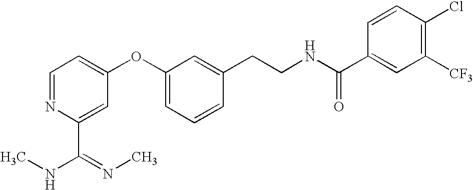

I-150

| | |
|---|---|
| 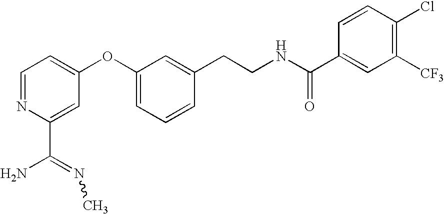 | 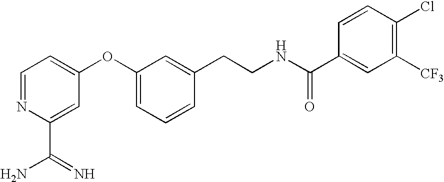 |
| I-151 | I-152 |
| 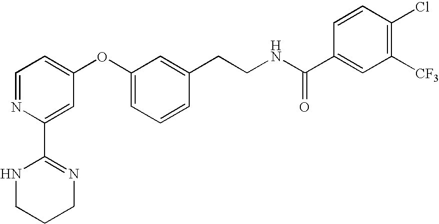 | 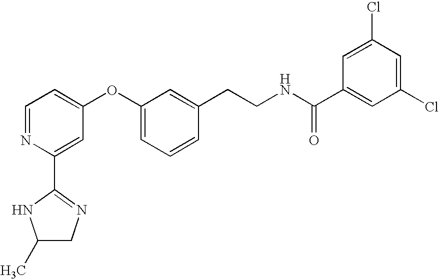 |
| I-153 | I-154 |

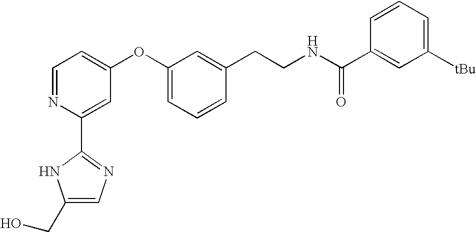

I-155

| | |
|---|---|
| 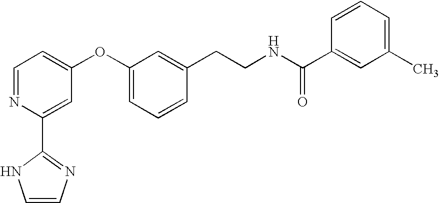 | 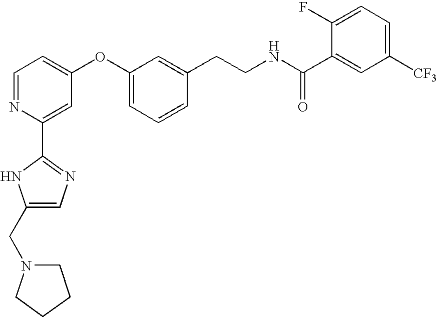 |

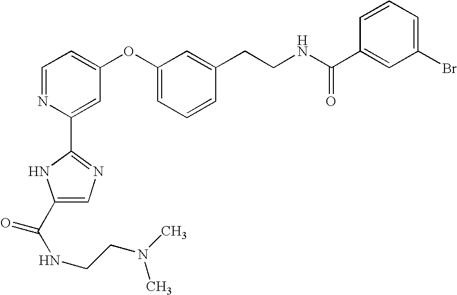

I-158

| | |
|---|---|
| 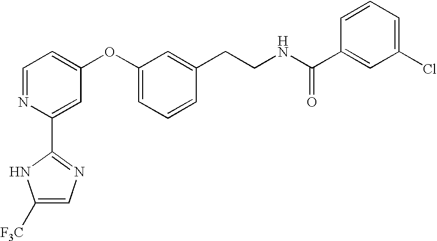 | 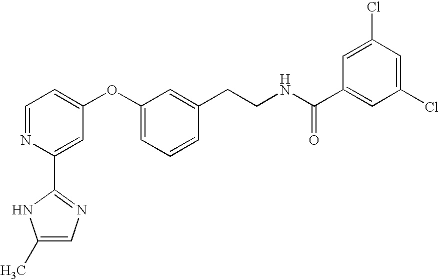 |
| I-159 | I-160 |
| 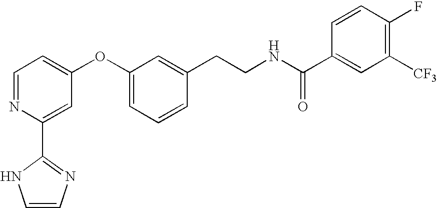 | 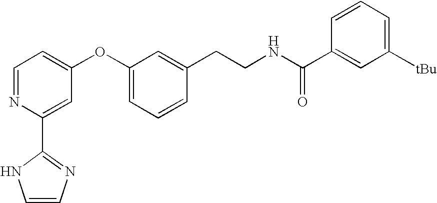 |
| I-161 | I-162 |

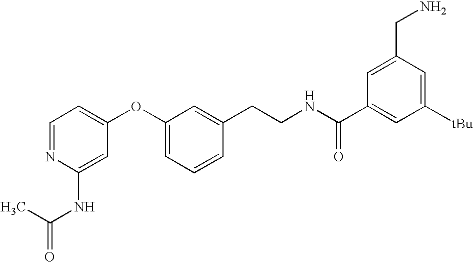

I-163

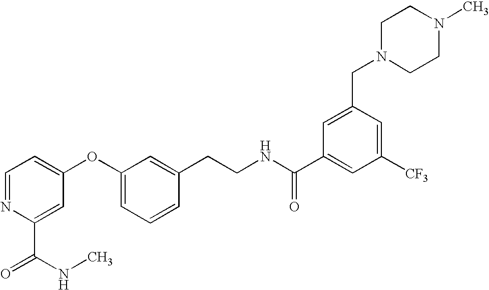

I-164

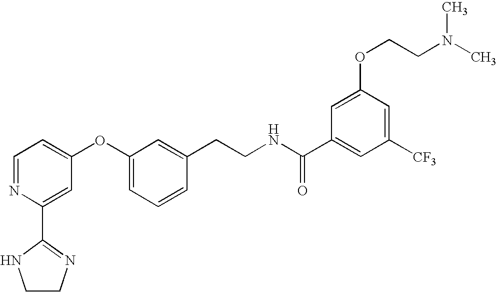

I-165

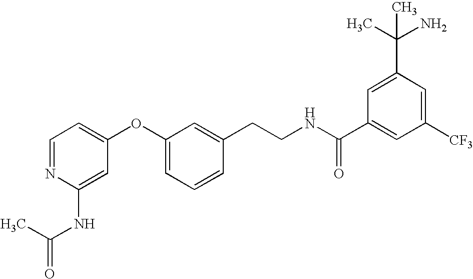

I-166

| | |
|---|---|
| 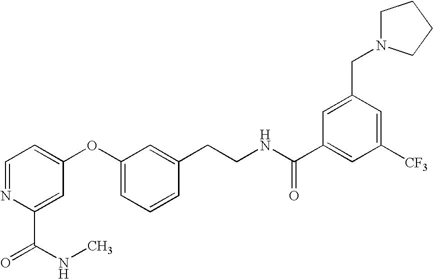 | 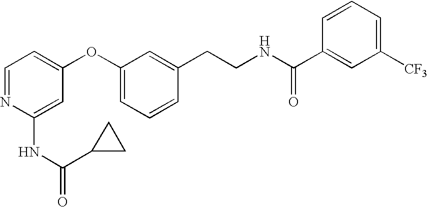 |
| I-167 | I-168 |
| 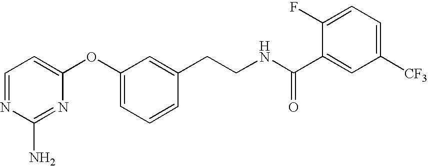 | 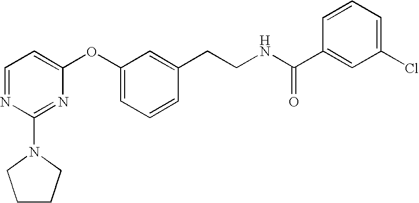 |
| I-169 | I-170 |
| 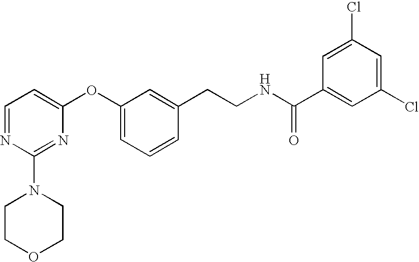 | 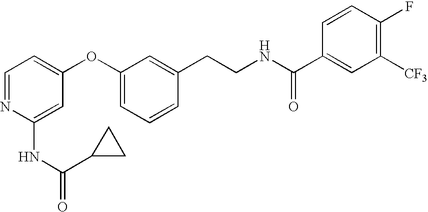 |
| I-171 | I-172 |
| 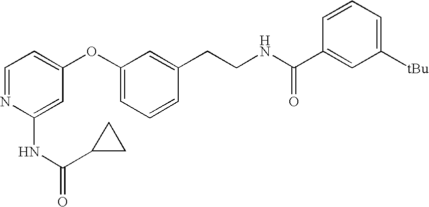 | 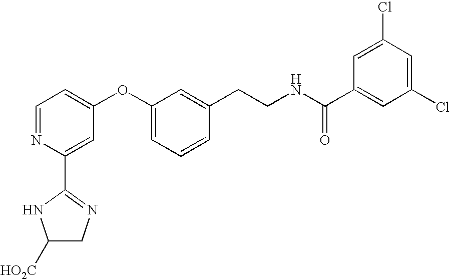 |
| I-173 | I-174 |

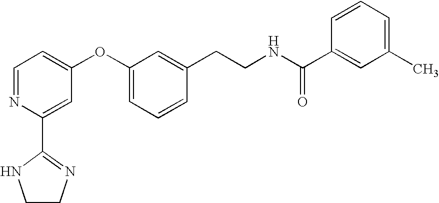

I-175

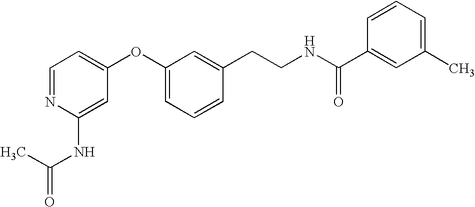

I-176

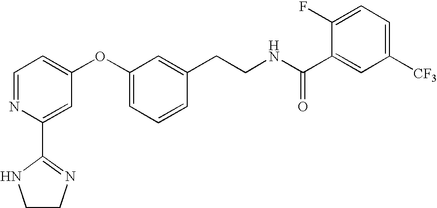

I-177

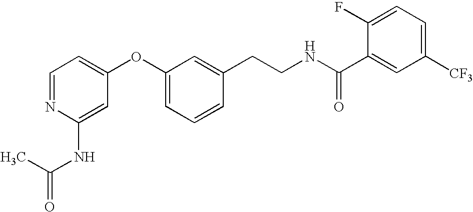

I-178

| | |
|---|---|
| 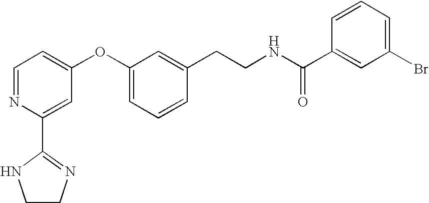 | 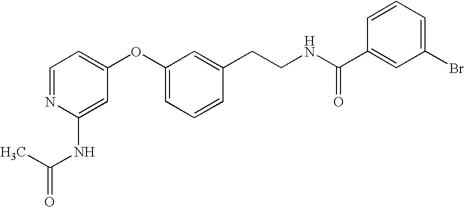 |
| I-179 | I-180 |
| 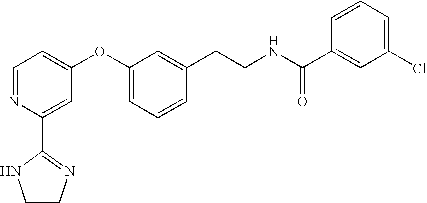 | 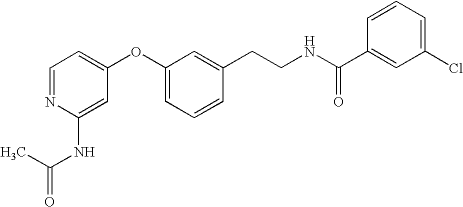 |
| I-181 | I-182 |
| 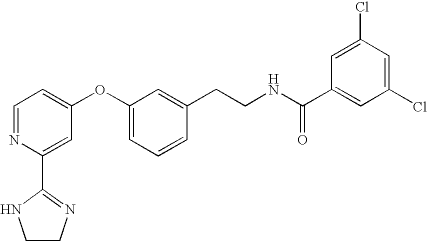 | 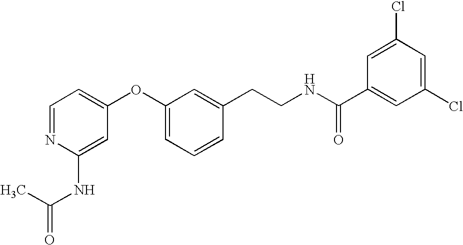 |
| I-183 | I-184 |

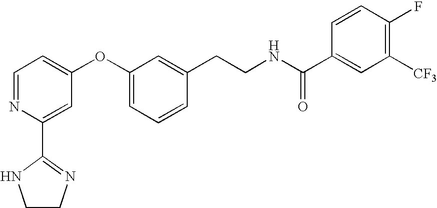

I-185

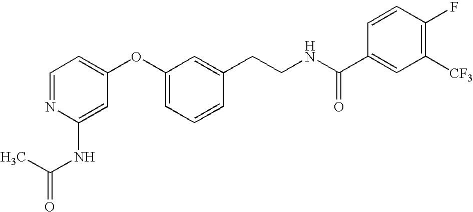

I-186

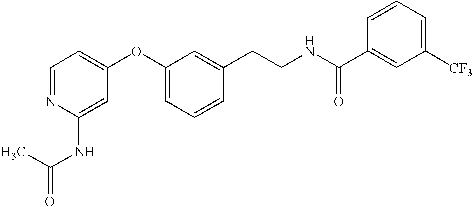

I-187

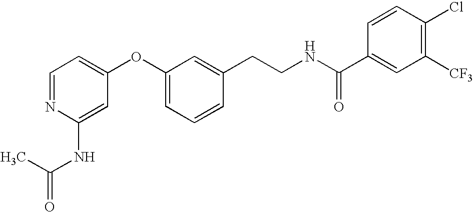

I-188

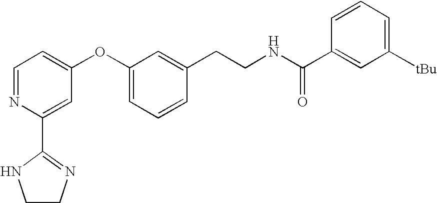

I-189

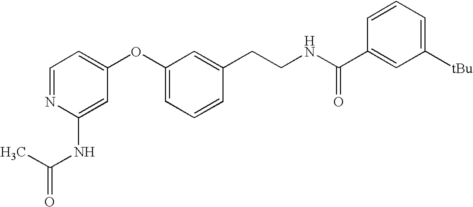

I-190

| | |
|---|---|
| 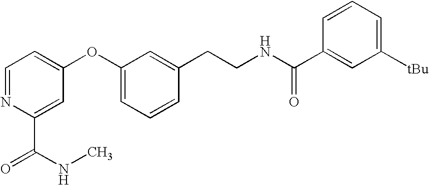 | 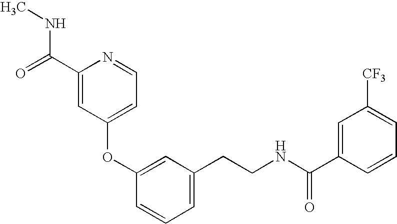 |
| I-191 | I-192 |
| 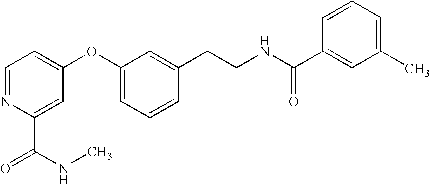 | 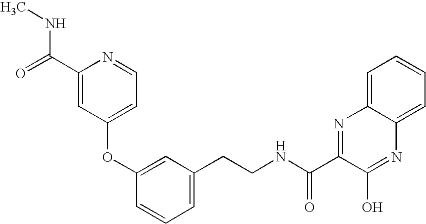 |
| I-193 | I-194 |

The compounds in Table 1 above also may be identified by the following chemical names:

| | Chemical Name |
|---|---|
| I-1 | 4-chloro-N-[2-(3-{[2-(4,5-dihydro-1H-imidazol-2-yl)pyridin-4-yl]oxy}phenyl)- |
| | ethyl]benzamide |
| I-2 | N-{2-[3-({2-[(4,5-dihydro-1H-imidazol-2-ylamino)methyl]pyridin-4-yl}- |
| | oxy)phenyl]ethyl}-3-(trifluoromethyl)benzamide |
| I-3 | 4-{3-[2-({[5-chloro-1-methyl-3-(trifluoromethyl)-1H-pyrazol-4-yl]carbonyl}- |
| | amino)ethyl]phenoxy}-N-methylpyridine-2-carboxamide |
| I-4 | 4-(3-{2-[(3-fluoro-5-morpholin-4-ylbenzoyl)amino]ethyl}phenoxy)-N- |
| | methylpyridine-2-carboxamide |
| I-5 | N-methyl-4-[3-(2-{[2-methyl-5-(pyrrolidin-1-ylsulfonyl)-3-furoyl]amino}ethyl)- |
| | phenoxy]pyridine-2-carboxamide |
| I-6 | 4-[3-(2-{[(5-bromo-4-methoxy-3-thienyl)carbonyl]amino}ethyl)phenoxy]-N- |
| | methylpyridine-2-carboxamide |
| I-7 | 4-[3-(2-{[3-(2-amino-1-methylethyl)benzoyl]amino}ethyl)phenoxy]-N- |
| | methylpyridine-2-carboxamide |
| I-8 | 4-(3-{2-[(3,5-dichlorobenzoyl)amino]ethyl}phenoxy)-N-methylpyridine-2- |
| | carboxamide |
| I-9 | 4-[3-(2-{[(3-tert-butyl-1-methyl-1H-pyrazol-5-yl)carbonyl]amino}ethyl)- |
| | phenoxy]-N-methylpyridine-2-carboxamide |
| I-10 | 4-[3-(2-{[(10,10-dioxido-9-oxo-9H-thioxanthen-3-yl)carbonyl]amino}ethyl)- |
| | phenoxy]-N-methylpyridine-2-carboxamide |
| I-11 | N-methyl-4-[3-(2-{[4-(1H-pyrazol-1-yl)benzoyl]amino}ethyl)phenoxy]pyridine- |
| | 2-carboxamide |
| I-12 | 4-[3-(2-{[4-chloro-3-(trifluoromethyl)benzoyl]amino}ethyl)phenoxy]-N- |
| | methylpyridine-2-carboxamide |
| I-13 | 4-[3-(2-{[3-chloro-2-fluoro-5-(trifluoromethyl)benzoyl]amino}ethyl)phenoxy]-N- |
| | methylpyridine-2-carboxamide |
| I-14 | 4-(3-{2-[(4-chloro-2-methoxybenzoyl)amino]ethyl}phenoxy)-N-methylpyridine- |
| | 2-carboxamide |
| I-15 | 4-(3-{2-[(3-chloro-4-methoxybenzoyl)amino]ethyl}phenoxy)-N-methylpyridine- |
| | 2-carboxamide |
| I-16 | N-{2-[3-({2-[(methylamino)carbonyl]pyridin-4-yl}oxy)phenyl]ethyl}quinoline-4- |
| | carboxamide |
| I-17 | N-methyl-4-[3-(2-{[(2-phenoxypyridin-3-yl)carbonyl]amino}ethyl)- |
| | phenoxy]pyridine-2-carboxamide |
| I-18 | 4-(3-{2-[(4-methoxy-2-methylbenzoyl)amino]ethyl}phenoxy)-N-methylpyridine- |
| | 2-carboxamide |
| I-19 | 4-(3-{2-[(4-methoxybenzoyl)amino]ethyl}phenoxy)-N-methylpyridine-2- |
| | carboxamide |
| I-20 | 4-(3-{2-[(4-methoxy-3-nitrobenzoyl)amino]ethyl}phenoxy)-N-methylpyridine-2- |
| | carboxamide |
| I-21 | N-[2-(3-{[2-(4,5-dihydro-1H-imidazol-2-yl)pyridin-4-yl]oxy}phenyl)ethyl]-3- |
| | fluoro-5-morpholin-4-ylbenzamide |
| I-22 | 4-[3-(2-{[4-(aminosulfonyl)benzoyl]amino}ethyl)phenoxy]-N-methylpyridine-2- |
| | carboxamide |
| I-23 | N-{2-[3-({2-[(methylamino)carbonyl]pyridin-4-yl}oxy)phenyl]ethyl}-1,3- |
| | benzothiazole-6-carboxamide |
| I-24 | N-methyl-4-[3-(2-{[4-(trifluoromethoxy)benzoyl]amino}ethyl)phenoxy]pyridine- |
| | 2-carboxamide |
| I-25 | 4-(3-{2-[(2,3-dimethoxybenzoyl)amino]ethyl}phenoxy)-N-methylpyridine-2- |
| | carboxamide |
| I-26 | N-methyl-4-[3-(2-{[3-(trifluoromethoxy)benzoyl]amino}ethyl)phenoxy]pyridine- |
| | 2-carboxamide |
| I-27 | 4-chloro-1,3-dimethyl-N-{2-[3-({2-[(methylamino)carbonyl]pyridin-4-yl}- |
| | oxy)phenyl]ethyl}-1H-pyrazolo[3,4-b]pyridine-5-carboxamide |
| I-28 | 4-{3-[2-({5-[(dimethylamino)sulfonyl]-2-methyl-3-furoyl}amino)ethyl]phenoxy}- |
| | N-methylpyridine-2-carboxamide |
| I-29 | N-methyl-4-[3-(2-{[5-methyl-2-(trifluoromethyl)-3-furoyl]amino}ethyl)- |
| | phenoxy]pyridine-2-carboxamide |
| I-30 | N-methyl-4-(3-{2-[(2,4,6-trifluorobenzoyl)amino]ethyl}phenoxy)pyridine-2- |
| | carboxamide |
| I-31 | 4-{3-[2-({2-[(2-cyanophenyl)sulfanyl]benzoyl}amino)ethyl]phenoxy}-N- |
| | methylpyridine-2-carboxamide |
| I-32 | 4-(3-{2-[(3-bromobenzoyl)amino]ethyl}phenoxy)-N-methylpyridine-2- |
| | carboxamide |
| I-33 | N-methyl-4-[3-(2-{[(4-methyl-2-pyridin-2-yl-1,3-thiazol-5-yl)carbonyl]- |
| | amino}ethyl)phenoxy]pyridine-2-carboxamide |
| I-34 | 4-(3-{2-[(4-cyanobenzoyl)amino]ethyl}phenoxy)-N-methylpyridine-2- |
| | carboxamide |
| I-35 | 4-(3-{2-[(4-chloro-2-fluorobenzoyl)amino]ethyl}phenoxy)-N-methylpyridine-2- |
| | carboxamide |
| I-36 | 1-ethyl-7-methyl-N-{2-[3-({2-[(methylamino)carbonyl]pyridin-4-yl}oxy)phenyl]- |
| | ethyl}-4-oxo-1,4-dihydro-1,8-naphthyridine-3-carboxamide |
| I-37 | 4-(3-{2-[(2-chloro-4,5-dimethoxybenzoyl)amino]ethyl}phenoxy)-N- |
| | methylpyridine-2-carboxamide |
| I-38 | N-{2-[3-({2-[(methylamino)carbonyl]pyridin-4-yl}oxy)phenyl]ethyl}-1- |
| | (phenylsulfonyl)-1H-indole-3-carboxamide |
| I-39 | 4-[3-(2-{[(3-ethyl-1-methyl-1H-pyrazol-5-yl)carbonyl]amino}ethyl)phenoxy]-N- |
| | methylpyridine-2-carboxamide |
| I-40 | 4-(3-{2-[(3-fluoro-4-methoxybenzoyl)amino]ethyl}phenoxy)-N-methylpyridine- |
| | 2-carboxamide |
| I-41 | 4-{3-[2-({[1-(2-chloro-6-fluorobenzyl)-2-oxo-1,2-dihydropyridin-3-yl]carbonyl}- |
| | amino)ethyl]phenoxy}-N-methylpyridine-2-carboxamide |
| I-42 | tert-butyl (2-{3-[({2-[3-({2-[(methylamino)carbonyl]pyridin-4-yl}oxy)phenyl]- |
| | ethyl}amino)carbonyl]phenyl}propyl)carbamate |
| I-43 | N-methyl-4-(3-{2-[(2-phenoxybenzoyl)amino]ethyl}phenoxy)pyridine-2- |
| | carboxamide |
| I-44 | 4-[3-(2-{[4-fluoro-3-(trifluoromethyl)benzoyl]amino}ethyl)phenoxy]-N- |
| | methylpyridine-2-carboxamide |
| I-45 | N-[2-(3-{[2-(aminomethyl)pyridin-4-yl]oxy}phenyl)ethyl]-3-(trifluoromethyl)- |
| | benzamide |
| I-46 | 4-[3-(2-{[(3′,4′-dichlorobiphenyl-4-yl)carbonyl]amino}ethyl)phenoxy]-N- |
| | methylpyridine-2-carboxamide |
| I-47 | 4-(3-{2-[(2,3-difluorobenzoyl)amino]ethyl}phenoxy)-N-methylpyridine-2- |
| | carboxamide |
| I-49 | 4-(3-{2-[(2-bromo-3-methylbenzoyl)amino]ethyl}phenoxy)-N-methylpyridine-2- |
| | carboxamide |
| I-50 | tert-butyl ({4-[3-(2-{[3-(trifluoromethyl)benzoyl]amino}ethyl)phenoxy]pyridin-2- |
| | yl}methyl)carbamate |
| I-51 | 4-[3-(2-{[3-(dimethylamino)benzoyl]amino}ethyl)phenoxy]-N-methylpyridine-2- |
| | carboxamide |
| I-52 | N-methyl-4-[3-(2-{[4-(methylsulfanyl)benzoyl]amino}ethyl)phenoxy]pyridine-2- |
| | carboxamide |
| I-53 | N-methyl-4-(3-{2-[(4-methyl-1-naphthoyl)amino]ethyl}phenoxy)pyridine-2- |
| | carboxamide |
| I-54 | 4-[3-(2-{[(5-chloro-4-methoxy-3-thienyl)carbonyl]amino}ethyl)phenoxy]-N- |
| | methylpyridine-2-carboxamide |
| I-55 | 4-[3-(2-{[(5-bromo-2,3-dihydro-1-benzofuran-7-yl)carbonyl]amino}ethyl)- |
| | phenoxy]-N-methylpyridine-2-carboxamide |
| I-56 | 4-(3-{2-[(3-ethoxybenzoyl)amino]ethyl}phenoxy)-N-methylpyridine-2- |
| | carboxamide |
| I-57 | N-[2-(3-{[2-(4,5-dihydro-1H-imidazol-2-yl)pyridin-4-yl]oxy}phenyl)ethyl]-3- |
| | (trifluoromethyl)benzamide |
| I-58 | N-{2-[3-({2-[(methylamino)carbonyl]pyridin-4-yl}oxy)phenyl]ethyl}-1,2,3- |
| | benzothiadiazole-5-carboxamide |
| I-59 | 4-(3-{2-[(4-methoxy-3-methylbenzoyl)amino]ethyl}phenoxy)-N-methylpyridine- |
| | 2-carboxamide |
| I-60 | 4-[3-(2-{[4-(diethylamino)benzoyl]amino}ethyl)phenoxy]-N-methylpyridine-2- |
| | carboxamide |
| I-61 | 4-(3-{2-[(4-chlorobenzoyl)amino]ethyl}phenoxy)-N-methylpyridine-2- |
| | carboxamide |
| I-62 | N-{2-[3-({2-[(methylamino)carbonyl]pyridin-4-yl}oxy)phenyl]ethyl}-2,1,3- |
| | benzothiadiazole-5-carboxamide |
| I-63 | 3-cyano-N-[2-(3-{[2-(4,5-dihydro-1H-imidazol-2-yl)pyridin-4-yl]oxy}phenyl)- |
| | ethyl]benzamide |
| I-64 | 4-(3-{2-[(5-chloro-2-methoxybenzoyl)amino]ethyl}phenoxy)-N-methylpyridine- |
| | 2-carboxamide |
| I-65 | 4-(3-{2-[(3,4-diethoxybenzoyl)amino]ethyl}phenoxy)-N-methylpyridine-2- |
| | carboxamide |
| I-66 | 4-[({2-[3-({2-[(methylamino)carbonyl]pyridin-4-yl}oxy)phenyl]ethyl}- |
| | amino)carbonyl]phenyl acetate |
| I-67 | 4-(3-{2-[(2,4-dimethoxybenzoyl)amino]ethyl}phenoxy)-N-methylpyridine-2- |
| | carboxamide |
| I-68 | 4-[4-(2-{[4-chloro-3-(trifluoromethyl)benzoyl]amino}ethyl)phenoxy]-N- |
| | methylpyridine-2-carboxamide |
| I-69 | 4-[3-(2-{[(2-methoxypyridin-3-yl)carbonyl]amino}ethyl)phenoxy]-N- |
| | methylpyridine-2-carboxamide |
| I-70 | N-methyl-4-{3-[2-({[5-(2-thienyl)pyridin-3-yl]carbonyl}amino)ethyl]- |
| | phenoxy}pyridine-2-carboxamide |
| I-71 | 4-(3-{2-[(4-bromo-2-methylbenzoyl)amino]ethyl}phenoxy)-N-methylpyridine-2- |
| | carboxamide |
| I-72 | 4-[3-(2-{[(3-chloro-1-benzothien-2-yl)carbonyl]amino}ethyl)phenoxy]-N- |
| | methylpyridine-2-carboxamide |
| I-73 | 4-[3-(2-{[4-(dimethylamino)benzoyl]amino}ethyl)phenoxy]-N-methylpyridine-2- |
| | carboxamide |
| I-74 | 4-(3-{2-[(2,4-dichlorobenzoyl)amino]ethyl}phenoxy)-N-methylpyridine-2- |
| | carboxamide |
| I-75 | 4-[3-(2-{[4-fluoro-2-(trifluoromethyl)benzoyl]amino}ethyl)phenoxy]-N- |
| | methylpyridine-2-carboxamide |
| I-76 | 4-(3-{2-[(4-isopropylbenzoyl)amino]ethyl}phenoxy)-N-methylpyridine-2- |
| | carboxamide |
| I-77 | N-methyl-4-[3-(2-{[(4-methyl-2-phenyl-1,3-thiazol-5-yl)carbonyl]amino}ethyl)- |
| | phenoxy]pyridine-2-carboxamide |
| I-78 | 4-(3-{2-[(2-methoxybenzoyl)amino]ethyl}phenoxy)-N-methylpyridine-2- |
| | carboxamide |
| I-79 | N-methyl-4-[3-(2-{[4-(1H-pyrrol-1-yl)benzoyl]amino}ethyl)phenoxy]pyridine-2- |
| | carboxamide |
| I-81 | 4-(3-{2-[(5-bromo-2-chlorobenzoyl)amino]ethyl}phenoxy)-N-methylpyridine-2- |
| | carboxamide |
| I-82 | 4-[3-(2-{[5-methoxy-2-(2,2,2-trifluoroethoxy)benzoyl]amino}ethyl)phenoxy]-N- |
| | methylpyridine-2-carboxamide |
| I-83 | N-methyl-4-[3-(2-{[(5-pyridin-2-yl-2-thienyl)carbonyl]amino}ethyl)- |
| | phenoxy]pyridine-2-carboxamide |
| I-84 | N-methyl-4-[3-(2-{[2-methyl-5-(piperidin-1-ylsulfonyl)-3-furoyl]amino}ethyl)- |
| | phenoxy]pyridine-2-carboxamide |
| I-85 | 4-(3-{2-[(3,5-dimethylbenzoyl)amino]ethyl}phenoxy)-N-methylpyridine-2- |
| | carboxamide |
| I-86 | N-methyl-4-(3-{2-[(3-phenoxybenzoyl)amino]ethyl}phenoxy)pyridine-2- |
| | carboxamide |
| I-87 | 4-(3-{2-[(3,4-difluorobenzoyl)amino]ethyl}phenoxy)-N-methylpyridine-2- |
| | carboxamide |
| I-88 | N-methyl-4-[3-(2-{[(2-methyl-1,3-thiazol-4-yl)carbonyl]amino}ethyl)- |
| | phenoxy]pyridine-2-carboxamide |
| I-89 | N-methyl-4-(3-{2-[(4-methylbenzoyl)amino]ethyl}phenoxy)pyridine-2- |
| | carboxamide |
| I-91 | N-{2-[3-({2-[(methylamino)carbonyl]pyridin-4-yl}oxy)phenyl]ethyl}-1H-indole- |
| | 5-carboxamide |
| I-92 | 4-chloro-N-[2-(3-{[2-(4,5-dihydro-1H-imidazol-2-yl)pyridin-4-yl]oxy}phenyl)- |
| | ethyl]-3-(trifluoromethyl)benzamide |
| I-93 | 4-(3-{2-[(2,6-difluorobenzoyl)amino]ethyl}phenoxy)-N-methylpyridine-2- |
| | carboxamide |
| I-94 | 4-(3-{2-[(3-methoxybenzoyl)amino]ethyl}phenoxy)-N-methylpyridine-2- |
| | carboxamide |
| I-95 | N-methyl-4-[3-(2-{[(5-methyl-2-thienyl)carbonyl]amino}ethyl)- |
| | phenoxy]pyridine-2-carboxamide |
| I-96 | 4-(3-{2-[(3,4-dimethylbenzoyl)amino]ethyl}phenoxy)-N-methylpyridine-2- |
| | carboxamide |
| I-98 | N-methyl-4-[3-(2-{[2-methyl-5-(morpholin-4-ylsulfonyl)-3-furoyl]amino}ethyl)- |
| | phenoxy]pyridine-2-carboxamide |
| I-99 | 4-{3-[2-({[2,5-dimethyl-1-(pyridin-4-ylmethyl)-1H-pyrrol-3-yl]carbonyl}- |
| | amino)ethyl]phenoxy}-N-methylpyridine-2-carboxamide |
| I-100 | N-methyl-4-{3-[2-({[6-(2,2,2-trifluoroethoxy)pyridin-3-yl]carbonyl}amino)ethyl]- |
| | phenoxy}pyridine-2-carboxamide |
| I-101 | 4-(3-{2-[(3-methoxy-2-methylbenzoyl)amino]ethyl}phenoxy)-N-methylpyridine- |
| | 2-carboxamide |
| I-102 | 4-(3-{2-[(2,5-dichlorobenzoyl)amino]ethyl}phenoxy)-N-methylpyridine-2- |
| | carboxamide |
| I-103 | 4-(3-{2-[(3,4-dimethoxybenzoyl)amino]ethyl}phenoxy)-N-methylpyridine-2- |
| | carboxamide |
| I-104 | 4-(3-{2-[(3-bromo-4-methylbenzoyl)amino]ethyl}phenoxy)-N-methylpyridine-2- |
| | carboxamide |
| I-105 | 4-{3-[2-({[5-(aminosulfonyl)-1-methyl-1H-pyrrol-2-yl]carbonyl}amino)ethyl]- |
| | phenoxy}-N-methylpyridine-2-carboxamide |
| I-106 | N-methyl-4-(3-{2-[(4-propylbenzoyl)amino]ethyl}phenoxy)pyridine-2- |
| | carboxamide |
| I-107 | N-[3-(3-{[2-(aminomethyl)pyridin-4-yl]oxy}phenyl)propyl]-4-chloro-3- |
| | (trifluoromethyl)benzamide |
| I-108 | 4-(3-{2-[(2,5-dimethyl-3-furoyl)amino]ethyl}phenoxy)-N-methylpyridine-2- |
| | carboxamide |
| I-109 | 4-(3-{2-[(biphenyl-2-ylcarbonyl)amino]ethyl}phenoxy)-N-methylpyridine-2- |
| | carboxamide |
| I-110 | 4-(3-{2-[(3-iodobenzoyl)amino]ethyl}phenoxy)-N-methylpyridine-2- |
| | carboxamide |
| I-111 | 4-methyl-N-{2-[3-({2-[(methylamino)carbonyl]pyridin-4-yl}oxy)phenyl]ethyl}-2- |
| | phenylpyrimidine-5-carboxamide |
| I-112 | methyl 4-[({2-[3-({2-[(methylamino)carbonyl]pyridin-4-yl}oxy)phenyl]ethyl}- |
| | amino)carbonyl]benzoate |
| I-114 | N-methyl-4-(3-{2-[(3,4,5-trimethoxybenzoyl)amino]ethyl}phenoxy)pyridine-2- |
| | carboxamide |
| I-115 | 4-(3-{2-[(3-chloro-4-fluorobenzoyl)amino]ethyl}phenoxy)-N-methylpyridine-2- |
| | carboxamide |
| I-116 | 4-(3-{2-[(3-chlorobenzoyl)amino]ethyl}phenoxy)-N-methylpyridine-2- |
| | carboxamide |
| I-117 | 4-[3-(2-{[(5-methoxy-1-benzofuran-2-yl)carbonyl]amino}ethyl)phenoxy]-N- |
| | methylpyridine-2-carboxamide |
| I-118 | N-methyl-4-(3-{2-[(2,4,5-trimethoxybenzoyl)amino]ethyl}phenoxy)pyridine-2- |
| | carboxamide |
| I-119 | 4-(3-{2-[(2,4-dichloro-5-fluorobenzoyl)amino]ethyl}phenoxy)-N-methylpyridine- |
| | 2-carboxamide |
| I-120 | 4-(3-{2-[(3-fluoro-4-methylbenzoyl)amino]ethyl}phenoxy)-N-methylpyridine-2- |
| | carboxamide |
| I-121 | 4-[3-(2-{[2-fluoro-3-(trifluoromethyl)benzoyl]amino}ethyl)phenoxy]-N- |
| | methylpyridine-2-carboxamide |
| I-122 | 6-methoxy-N-{2-[3-({2-[(methylamino)carbonyl]pyridin-4-yl}oxy)phenyl]ethyl}- |
| | 2-phenylquinoline-4-carboxamide |
| I-124 | 4-(3-{2-[(2-chloro-4,5-difluorobenzoyl)amino]ethyl}phenoxy)-N-methylpyridine- |
| | 2-carboxamide |
| I-125 | N-methyl-4-[3-(2-{[(2-phenyl-1,3-thiazol-4-yl)carbonyl]amino}ethyl)- |
| | phenoxy]pyridine-2-carboxamide |
| I-126 | N-[2-(3-{[2-(4,5-dihydro-1H-imidazol-2-yl)pyridin-4-yl]oxy}phenyl)ethyl]-3- |
| | (trifluoromethoxy)benzamide |
| I-127 | N-methyl-4-[3-(2-{[(1-methyl-1H-pyrrol-2-yl)carbonyl]amino}ethyl)- |
| | phenoxy]pyridine-2-carboxamide |
| I-128 | 4-chloro-N-[3-(3-{[2-(4,5-dihydro-1H-imidazol-2-yl)pyridin-4-yl]oxy}phenyl)- |
| | propyl]-3-(trifluoromethyl)benzamide |
| I-129 | 4-[4-(3-{[4-chloro-3-(trifluoromethyl)benzoyl]amino}propyl)phenoxy]-N- |
| | methylpyridine-2-carboxamide |
| I-130 | N-methyl-4-[3-(2-{[3-(1H-tetrazol-1-yl)benzoyl]amino}ethyl)phenoxy]pyridine- |
| | 2-carboxamide |
| I-131 | 4-[3-(2-{[(4,5-dichloroisothiazol-3-yl)carbonyl]amino}ethyl)phenoxy]-N- |
| | methylpyridine-2-carboxamide |
| I-132 | 4-[3-(2-{[3-fluoro-5-(trifluoromethyl)benzoyl]amino}ethyl)phenoxy]-N- |
| | methylpyridine-2-carboxamide |
| I-133 | 4-(3-{2-[(4-fluoro-3-methylbenzoyl)amino]ethyl}phenoxy)-N-methylpyridine-2- |
| | carboxamide |
| I-134 | N-methyl-4-[3-(2-{[3-(piperazin-1-ylmethyl)-5-(trifluoromethyl)benzoyl]- |
| | amino}ethyl)phenoxy]pyridine-2-carboxamide |
| I-135 | N-(2-{3-[(2-{5-[(4-methylpiperazin-1-yl)carbonyl]-1H-imidazol-2-yl}pyridin-4- |
| | yl)oxy]phenyl}ethyl)-3-(trifluoromethyl)benzamide |
| I-136 | N-{2-[3-({2-[5-(piperazin-1-ylmethyl)-1H-imidazol-2-yl]pyridin-4-yl}- |
| | oxy)phenyl]ethyl}-3-(trifluoromethyl)benzamide |
| I-137 | N-methyl-4-{[3-(2-{[3-(trifluoromethyl)benzoyl]amino}ethyl)phenyl]- |
| | amino}pyridine-2-carboxamide |
| I-138 | N-methyl-4-[3-(2-{[3-(trifluoromethyl)benzoyl]amino}ethyl)benzyl]pyridine-2- |
| | carboxamide |
| I-139 | N-methyl-4-{[3-(2-{[3-(trifluoromethyl)benzoyl]amino}ethyl)phenyl]sulfinyl}- |
| | pyridine-2-carboxamide |
| I-140 | N-methyl-4-{[3-(2-{[3-(trifluoromethyl)benzoyl]amino}ethyl)phenyl]sulfanyl}- |
| | pyridine-2-carboxamide |
| I-141 | N-methyl-4-[3-(2-methyl-2-{[3-(trifluoromethyl)benzoyl]amino}propyl)- |
| | phenoxy]pyridine-2-carboxamide |
| I-142 | N-{2-[3-({2-[5-(morpholin-4-ylmethyl)-1H-imidazol-2-yl]pyridin-4-yl}- |
| | oxy)phenyl]ethyl}-3-(trifluoromethyl)benzamide |
| I-143 | 4-[3-fluoro-5-(2-{[3-(trifluoromethyl)benzoyl]amino}ethyl)phenoxy]-N- |
| | methylpyridine-2-carboxamide |
| I-144 | N-(2-{3-[(7-oxo-5,6,7,8-tetrahydro-1,8-naphthyridin-4-yl)oxy]phenyl}ethyl)-3- |
| | (trifluoromethyl)benzamide |
| I-145 | N-[2-(3-{[2-(1-methyl-1H-pyrazol-4-yl)pyridin-4-yl]oxy}phenyl)ethyl]-3- |
| | (trifluoromethyl)benzamide |
| I-146 | 4-[3-(2-{[3-[(dimethylamino)methyl]-5-(trifluoromethyl)benzoyl]amino}ethyl)- |
| | phenoxy]-N-methylpyridine-2-carboxamide |
| I-147 | 4-[4-fluoro-3-(2-{[3-(trifluoromethyl)benzoyl]amino}ethyl)phenoxy]-N- |
| | methylpyridine-2-carboxamide |
| I-148 | N-methyl-4-{[2-(2-{[3-(trifluoromethyl)benzoyl]amino}ethyl)pyridin-4-yl]- |
| | oxy}pyridine-2-carboxamide |
| I-149 | N-[2-(3-{[2-(2-methyl-1,3-oxazol-5-yl)pyridin-4-yl]oxy}phenyl)ethyl]-3- |
| | (trifluoromethyl)benzamide |
| I-150 | 4-chloro-N-{2-[3-({2-[(E)-(methylamino)(methylimino)methyl]pyridin-4-yl}- |
| | oxy)phenyl]ethyl}-3-(trifluoromethyl)benzamide |
| I-151 | N-{2-[3-({2-[(Z)-amino(methylimino)methyl]pyridin-4-yl}oxy)phenyl]ethyl}-4- |
| | chloro-3-(trifluoromethyl)benzamide |
| I-152 | N-{2-[3-({2-[amino(imino)methyl]pyridin-4-yl}oxy)phenyl]ethyl}-4-chloro-3- |
| | (trifluoromethyl)benzamide |
| I-153 | 4-chloro-N-[2-(3-{[2-(1,4,5,6-tetrahydropyrimidin-2-yl)pyridin-4-yl]oxy}phenyl)- |
| | ethyl]-3-(trifluoromethyl)benzamide |
| I-154 | 3,5-dichloro-N-[2-(3-{[2-(5-methyl-4,5-dihydro-1H-imidazol-2-yl)pyridin-4-yl]- |
| | oxy}phenyl)ethyl]benzamide |
| I-155 | 3-tert-butyl-N-{2-[3-({2-[5-(hydroxymethyl)-1H-imidazol-2-yl]pyridin-4-yl}- |
| | oxy)phenyl]ethyl}benzamide |
| I-156 | N-[2-(3-{[2-(1H-imidazol-2-yl)pyridin-4-yl]oxy}phenyl)ethyl]-3- |
| | methylbenzamide |
| I-157 | 2-fluoro-N-{2-[3-({2-[5-(pyrrolidin-1-ylmethyl)-1H-imidazol-2-yl]pyridin-4-yl}- |
| | oxy)phenyl]ethyl}-5-(trifluoromethyl)benzamide |
| I-158 | 2-[4-(3-{2-[(3-bromobenzoyl)amino]ethyl}phenoxy)pyridin-2-yl]-N-[2- |
| | (dimethylamino)ethyl]-1H-imidazole-5-carboxamide |
| I-159 | 3-chloro-N-{2-[3-({2-[5-(trifluoromethyl)-1H-imidazol-2-yl]pyridin-4-yl}- |
| | oxy)phenyl]ethyl}benzamide |
| I-160 | 3,5-dichloro-N-[2-(3-{[2-(5-methyl-1H-imidazol-2-yl)pyridin-4-yl]oxy}phenyl)- |
| | ethyl]benzamide |
| I-161 | 4-fluoro-N-[2-(3-{[2-(1H-imidazol-2-yl)pyridin-4-yl]oxy}phenyl)ethyl]-3- |
| | (trifluoromethyl)benzamide |
| I-162 | 3-tert-butyl-N-[2-(3-{[2-(1H-imidazol-2-yl)pyridin-4-yl]oxy}phenyl)ethyl]- |
| | benzamide |
| I-163 | N-[2-(3-{[2-(acetylamino)pyridin-4-yl]oxy}phenyl)ethyl]-3-(aminomethyl)-5-tert- |
| | butylbenzamide |
| I-164 | N-methyl-4-[3-(2-{[3-[(4-methylpiperazin-1-yl)methyl]-5-(trifluoromethyl)- |
| | benzoyl]amino}ethyl)phenoxy]pyridine-2-carboxamide |
| I-165 | N-[2-(3-{[2-(4,5-dihydro-1H-imidazol-2-yl)pyridin-4-yl]oxy}phenyl)ethyl]-3-[2- |
| | (dimethylamino)ethoxy]-5-(trifluoromethyl)benzamide |
| I-166 | N-[2-(3-{[2-(acetylamino)pyridin-4-yl]oxy}phenyl)ethyl]-3-(1-amino-1- |
| | methylethyl)-5-(trifluoromethyl)benzamide |
| I-167 | N-methyl-4-[3-(2-{[3-(pyrrolidin-1-ylmethyl)-5-(trifluoromethyl)benzoyl]- |
| | amino}ethyl)phenoxy]pyridine-2-carboxamide |
| I-168 | N-{2-[3-({2-[(cyclopropylcarbonyl)amino]pyridin-4-yl}oxy)phenyl]ethyl}-3- |
| | (trifluoromethyl)benzamide |
| I-169 | N-(2-{3-[(2-aminopyrimidin-4-yl)oxy]phenyl}ethyl)-2-fluoro-5-(trifluoromethyl)- |
| | benzamide |
| I-170 | 3-chloro-N-(2-{3-[(2-pyrrolidin-1-ylpyrimidin-4-yl)oxy]phenyl}ethyl)benzamide |
| I-171 | 3,5-dichloro-N-(2-{3-[(2-morpholin-4-ylpyrimidin-4-yl)oxy]phenyl}ethyl)- |
| | benzamide |
| I-172 | N-{2-[3-({2-[(cyclopropylcarbonyl)amino]pyridin-4-yl}oxy)phenyl]ethyl}-4- |
| | fluoro-3-(trifluoromethyl)benzamide |
| I-173 | 3-tert-butyl-N-{2-[3-({2-[(cyclopropylcarbonyl)amino]pyridin-4-yl}oxy)phenyl]- |
| | ethyl}benzamide |
| I-174 | 2-[4-(3-{2-[(3,5-dichlorobenzoyl)amino]ethyl}phenoxy)pyridin-2-yl]-4,5- |
| | dihydro-1H-imidazole-5-carboxylic acid |
| I-175 | N-[2-(3-{[2-(4,5-dihydro-1H-imidazol-2-yl)pyridin-4-yl]oxy}phenyl)ethyl]-3- |
| | methylbenzamide |
| I-176 | N-[2-(3-{[2-(acetylamino)pyridin-4-yl]oxy}phenyl)ethyl]-3-methylbenzamide |
| I-177 | N-[2-(3-{[2-(4,5-dihydro-1H-imidazol-2-yl)pyridin-4-yl]oxy}phenyl)ethyl]-2- |
| | fluoro-5-(trifluoromethyl)benzamide |
| I-178 | N-[2-(3-{[2-(acetylamino)pyridin-4-yl]oxy}phenyl)ethyl]-2-fluoro-5- |
| | (trifluoromethyl)benzamide |
| I-179 | 3-bromo-N-[2-(3-{[2-(4,5-dihydro-1H-imidazol-2-yl)pyridin-4-yl]oxy}phenyl)- |
| | ethyl]benzamide |
| I-180 | N-[2-(3-{[2-(acetylamino)pyridin-4-yl]oxy}phenyl)ethyl]-3-bromobenzamide |
| I-181 | 3-chloro-N-[2-(3-{[2-(4,5-dihydro-1H-imidazol-2-yl)pyridin-4-yl]oxy}phenyl)- |
| | ethyl]benzamide |
| I-182 | N-[2-(3-{[2-(acetylamino)pyridin-4-yl]oxy}phenyl)ethyl]-3-chlorobenzamide |
| I-183 | 3,5-dichloro-N-[2-(3-{[2-(4,5-dihydro-1H-imidazol-2-yl)pyridin-4-yl]- |
| | oxy}phenyl)ethyl]benzamide |
| I-184 | N-[2-(3-{[2-(acetylamino)pyridin-4-yl]oxy}phenyl)ethyl]-3,5-dichlorobenzamide |
| I-185 | N-[2-(3-{[2-(4,5-dihydro-1H-imidazol-2-yl)pyridin-4-yl]oxy}phenyl)ethyl]-4- |
| | fluoro-3-(trifluoromethyl)benzamide |
| I-186 | N-[2-(3-{[2-(acetylamino)pyridin-4-yl]oxy}phenyl)ethyl]-4-fluoro-3- |
| | (trifluoromethyl)benzamide |
| I-187 | N-[2-(3-{[2-(acetylamino)pyridin-4-yl]oxy}phenyl)ethyl]-3-(trifluoromethyl)- |
| | benzamide |
| I-188 | N-[2-(3-{[2-(acetylamino)pyridin-4-yl]oxy}phenyl)ethyl]-4-chloro-3- |
| | (trifluoromethyl)benzamide |
| I-189 | 3-tert-butyl-N-[2-(3-{[2-(4,5-dihydro-1H-imidazol-2-yl)pyridin-4-yl]oxy}phenyl)- |
| | ethyl]benzamide |
| I-190 | N-[2-(3-{[2-(acetylamino)pyridin-4-yl]oxy}phenyl)ethyl]-3-tert-butylbenzamide |
| I-191 | 4-(3-{2-[(3-tert-butylbenzoyl)amino]ethyl}phenoxy)-N-methylpyridine-2- |
| | carboxamide |
| I-192 | N-methyl-4-[3-(2-{[3-(trifluoromethyl)benzoyl]amino}ethyl)phenoxy]pyridine-2- |
| | carboxamide |
| I-193 | N-methyl-4-(3-{2-[(3-methylbenzoyl)amino]ethyl}phenoxy)pyridine-2- |
| | carboxamide |
| I-194 | 3-hydroxy-N-{2-[3-({2-[(methylamino)carbonyl]pyridin-4-yl}oxy)- |
| | phenyl]ethyl}quinoxaline-2-carboxamide |

General Synthetic Methodology

The compounds of the present invention can be prepared by methods known to one of ordinary skill in the art and/or by reference to the schemes shown below and the synthetic examples that follow. Exemplary synthetic routes are set forth in Schemes below, and in the Examples.

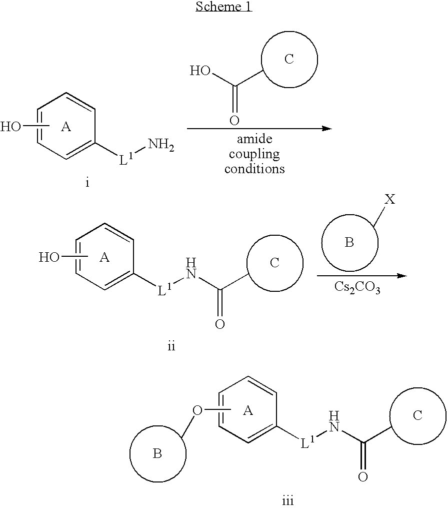

In general, compounds of formula (I) wherein G is —O— can be prepared as depicted in Scheme 1. Aminophenol i is combined with a carboxylic acid under standard amide bond forming conditions to give amide ii. Treatment of ii with a heterocyclic halide or nitro-containing compound in the presence of DMF and cesium carbonate or other base then provides biaryl ether iii.

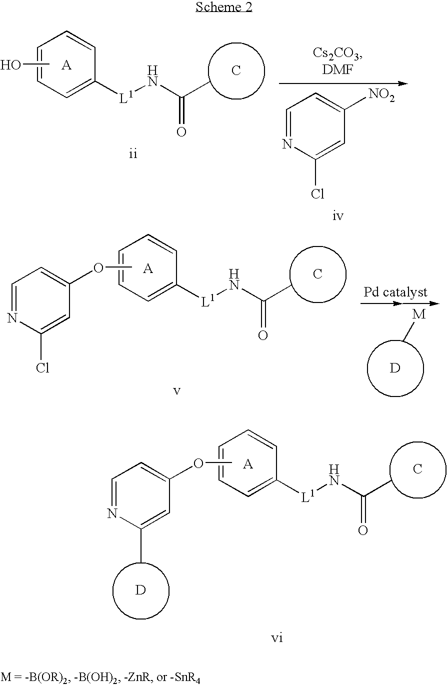

Compounds of formula (III), wherein G is —O— and Ring D is heteroaryl, can be prepared as outlined in Scheme 2. Phenol ii is combined with nitro chloropyridine iv in warm DMF and cesium carbonate. The resulting chloropyridine v is then coupled with a heteroaryl reagent in the presence of a palladium catalyst under Stille, Suzuki, or Negishi conditions to provide the biaryl ether vi.

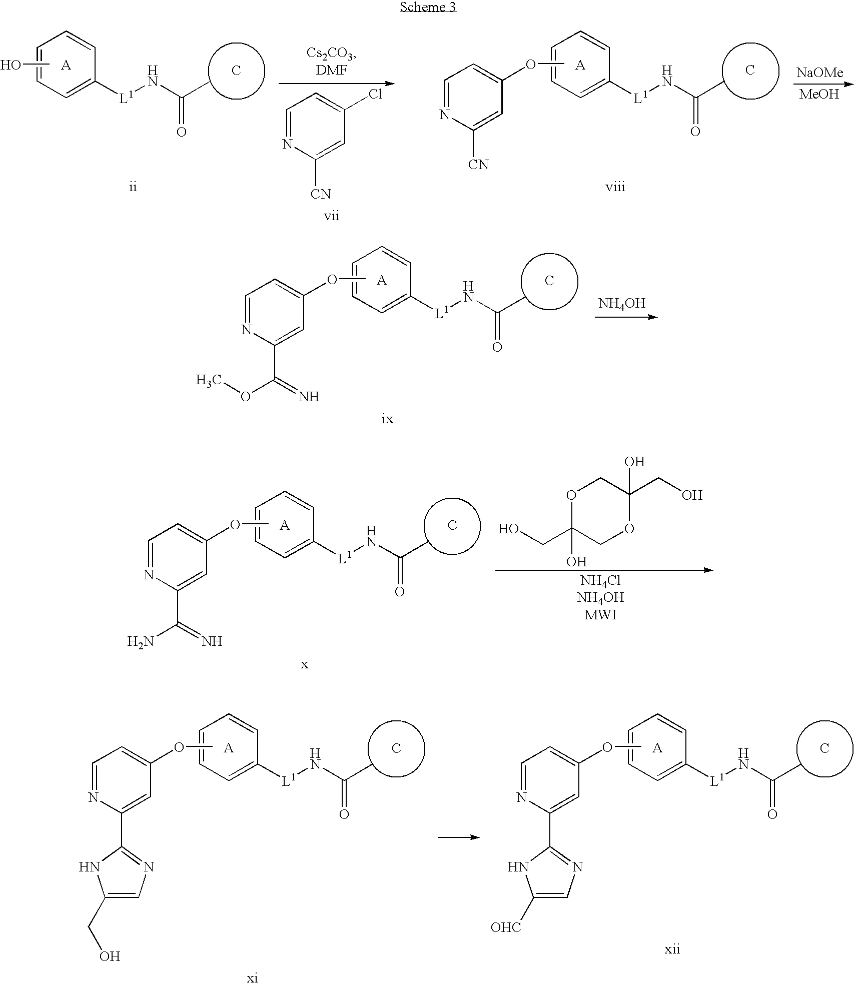

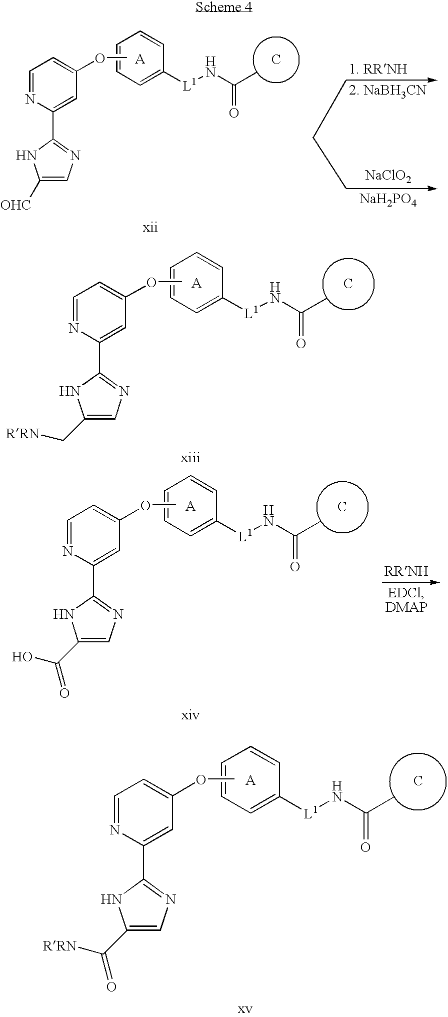

Alternatively, compounds wherein Ring D is a substituted imidazole can be prepared from the cyanopyridine compound viii, itself the result of heating phenol ii and chlorocyanopyridine vii in the presence of base in DMF (Scheme 3). The resultant cyanopyridine viii is then converted to acyclic amidine x via the imidate ix, using standard conditions. Treatment of amidine x with hydroxyacetone dimer and microwave irradiation provides hydroxy imidazole xi, which can be oxidized using Dess-Martin reagent or manganese dioxide to give aldehyde xii. Aldehyde xii can be combined with an amine under standard reductive alkylation conditions to give aminoalkyl imidazoles xi ii, or it can be further oxidized to the acid xiv and then coupled under standard amide bond forming conditions to give amides xv (Scheme 4).

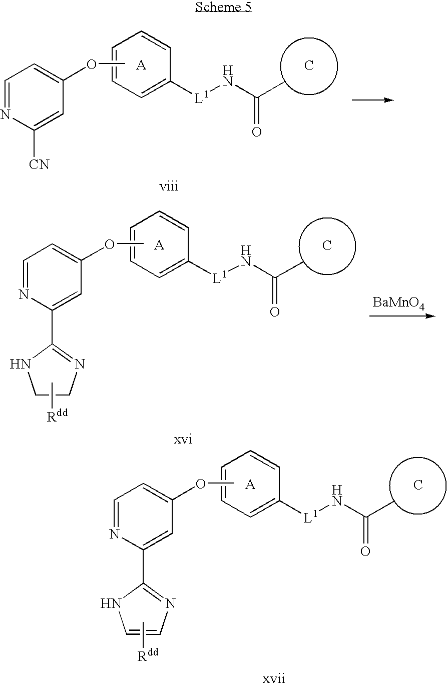

As depicted in Scheme 5, cyanopyridine viii also can be converted to cyclic amidines by treatment with hydrogen sulfide gas, followed by a diamine in the presence of ethanol and triethyl amine. Oxidation of the resultant amidine xvi with BaMnO₄ provides imidazoles xvii.

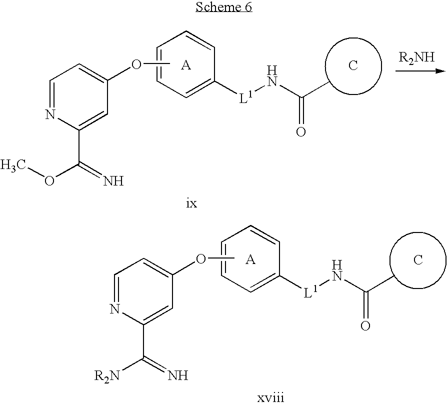

Substituted acyclic amidines xviii can be prepared from imidate ix by heating in the presence of an amine and triethyl amine (Scheme 6).

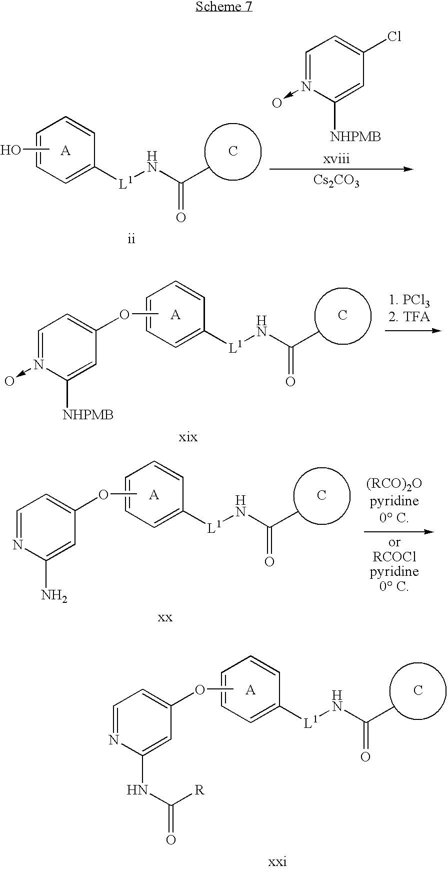

Aminopyridines can be prepared by reacting phenol ii with the PMB-protected pyridine xviii in the presence of cesium carbonate in DMF (Scheme 7). Deprotection of the amino pyridine with PCl₃ and trifluoroacetic acid provides amino pyridine xx, which can be further acylated by treatment with either an anhydride or acid chloride in pyridine at 0° C.

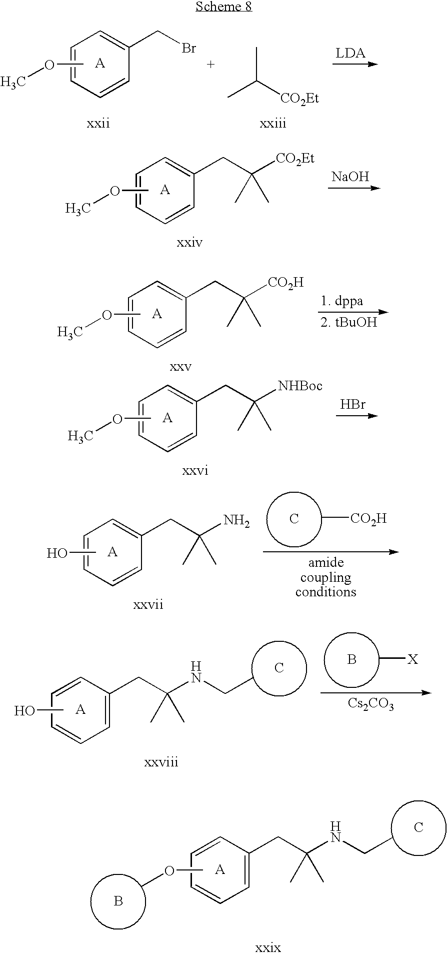

Compounds in which the linker L¹ is substituted (i.e. R^{j} and R^{k}=Me) can be prepared as outlined in Scheme 8. Thus, alkylation of ester xxiii with benzyl bromide xxii (as described by Mueller et al. J. Med. Chem. 2004, 47, 5183) provides ester xxiv. Ester hydrolysis, Curtius rearrangement, and boc deprotection provides amine xxvii. Amide bond coupling and ether bond formation then provides amides xxix.

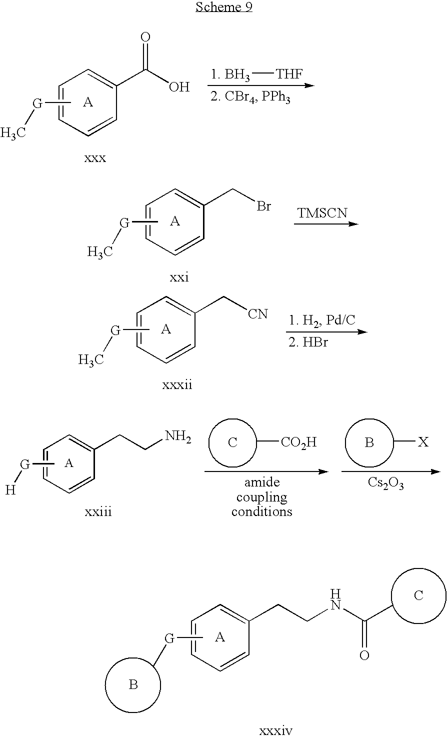

Compounds in which G is —S— or —NH— can be prepared as shown in Scheme 9. Acid xxx (where G=S or N) is reduced to a benzyl alcohol and then converted to bromide xxxi with carbon tetrabromide. Treatment of the bromide with TMSCN provides nitrile xxxii, which is then reduced under hydrogen in the presence of palladium and deprotected with HBr to give amine xxxiii (G=S, N). Amide coupling and ether bond formation provides biaryl ether xxxiv.

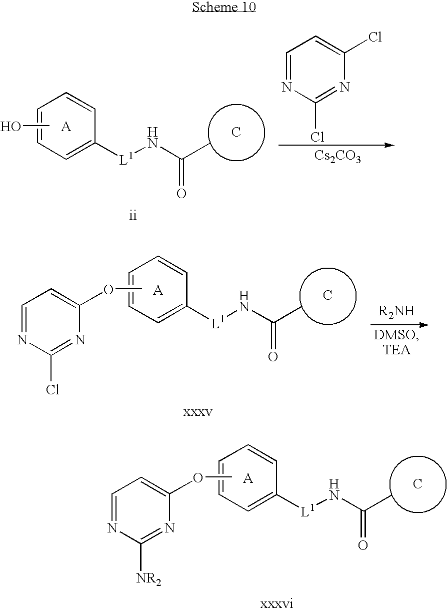

Compounds in which Ring B is an aminopyrimidine can be prepared as shown in Scheme 10. Phenol ii is treated first with 2,4-dichloropyrimidine in the presence of cesium carbonate and DMF. The resulting biaryl ether xxxv is then heated in DMSO in the presence of triethylamine and a primary or secondary amine to provide aminopyrimidine xxxvi.

Uses, Formulation, and Administration

As discussed above, the present invention provides compounds that are inhibitors of Raf kinases. The compounds can be assayed in vitro or in vivo for their ability to bind to and/or inhibit a Raf kinase. In vitro assays include assays to determine inhibition of the ability of the kinase to phosphorylate a substrate protein or peptide. Alternate in vitro assays quantitate the ability of the compound to bind to the kinase. Inhibitor binding may be measured by radiolabelling the inhibitor prior to binding, isolating the inhibitor/kinase complex and determining the amount of radiolabel bound. Alternatively, inhibitor binding may be determined by running a competition experiment in which new inhibitors are incubated with the kinase bound to a known radioligand. The compounds also can be assayed for their ability to affect cellular or physiological functions mediated by protein kinase activity. Assays for each of these activities are described in the Examples and/or are known in the art.

In another aspect, therefore, the invention provides a method for inhibiting Raf kinase activity in a cell, comprising contacting a cell in which inhibition of a Raf kinase is desired with a compound of formula (I). In some embodiments, the compound of formula (I) interacts with and reduces the activity of more than one Raf kinase enzyme in the cell. By way of example, when assayed against B-Raf and C-Raf, some compounds of formula (I) show inhibition of both enzymes. In some embodiments, the compound of formula (I) is selective, i.e., the concentration of the compound that is required for inhibition of one Raf kinase enzymes is lower, preferably at least 2-fold, 5-fold, 10-fold, or 50-fold lower, than the concentration of the compound required for inhibition of another Raf kinase enzyme.

In some embodiments, the compound of formula (I) inhibits one or more Raf kinase enzymes at a concentration that is lower than the concentration of the compound required for inhibition of other, unrelated, kinase enzymes. In some other embodiments, in addition to inhibiting Raf kinase, the compound formula (I) also inhibits one or more other kinase enzymes, preferably other kinase enzymes involved in tumor cell proliferation.

The invention thus provides a method for inhibiting cell proliferation, comprising contacting a cell in which such inhibition is desired with a compound of formula (I). The phrase “inhibiting cell proliferation” is used to denote the ability of a compound of formula (I) to inhibit cell number or cell growth in contacted cells as compared to cells not contacted with the inhibitor. An assessment of cell proliferation can be made by counting cells using a cell counter or by an assay of cell viability, e.g., an MTT or WST assay. Where the cells are in a solid growth (e.g., a solid tumor or organ), such an assessment of cell proliferation can be made by measuring the growth, e.g., with calipers, and comparing the size of the growth of contacted cells with non-contacted cells.

Preferably, the growth of cells contacted with the inhibitor is retarded by at least about 50% as compared to growth of non-contacted cells. In various embodiments, cell proliferation of contacted cells is inhibited by at least about 75%, at least about 90%, or at least about 95% as compared to non-contacted cells. In some embodiments, the phrase “inhibiting cell proliferation” includes a reduction in the number of contacted cells, as compare to non-contacted cells. Thus, a kinase inhibitor that inhibits cell proliferation in a contacted cell may induce the contacted cell to undergo growth retardation, to undergo growth arrest, to undergo programmed cell death (i.e., apoptosis), or to undergo necrotic cell death.

In another aspect, the invention provides a pharmaceutical composition comprising a compound of formula (I) as defined above, or a pharmaceutically acceptable salt thereof, and a pharmaceutically acceptable carrier.

If pharmaceutically acceptable salts of the compounds of the invention are utilized in these compositions, the salts preferably are derived from inorganic or organic acids and bases. For reviews of suitable salts, see, e.g., Berge et al, J. Pharm. Sci. 66:1-19 (1977) and Remington: The Science and Practice of Pharmacy, 20th Ed., ed. A. Gennaro, Lippincott Williams & Wilkins, 2000.

Nonlimiting examples of suitable acid addition salts include the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, formate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

Suitable base addition salts include, without limitation, ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

Also, basic nitrogen-containing groups may be quaternized with such agents as lower alkyl halides, such as methyl, ethyl, propyl, and butyl chloride, bromides and iodides; dialkyl sulfates, such as dimethyl, diethyl, dibutyl and diamyl sulfates, long chain halides such as decyl, lauryl, myristyl and stearyl chlorides, bromides and iodides, aralkyl halides, such as benzyl and phenethyl bromides and others. Water or oil-soluble or dispersible products are thereby obtained.

The term “pharmaceutically acceptable carrier” is used herein to refer to a material that is compatible with a recipient subject, preferably a mammal, more preferably a human, and is suitable for delivering an active agent to the target site without terminating the activity of the agent. The toxicity or adverse effects, if any, associated with the carrier preferably are commensurate with a reasonable risk/benefit ratio for the intended use of the active agent.

The pharmaceutical compositions of the invention can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain stabilizers, pH modifiers, surfactants, bioavailability modifiers and combinations of these.

Pharmaceutical formulations may be prepared as liquid suspensions or solutions using a liquid, such as, but not limited to, an oil, water, an alcohol, and combinations of these. Pharmaceutically suitable surfactants, suspending agents, or emulsifying agents, may be added for oral or parenteral administration. Suspensions may include oils, such as but not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

According to a preferred embodiment, the compositions of this invention are formulated for pharmaceutical administration to a mammal, preferably a human being. Such pharmaceutical compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intravenously, or subcutaneously. The formulations of the invention may be designed to be short-acting, fast-releasing, or long-acting. Still further, compounds can be administered in a local rather than systemic means, such as administration (e.g., by injection) at a tumor site.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers.

The pharmaceutical compositions of this invention may be orally administered in any orally acceptable dosage form including, but not limited to, capsules, tablets, aqueous suspensions or solutions. In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These may be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract may be effected in a rectal suppository formulation (see above) or in a suitable enema formulation. Topically-transdermal patches may also be used. For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions may be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The pharmaceutical compositions of the invention preferably are formulated for administration to a patient having, or at risk of developing or experiencing a recurrence of, a Raf kinase-mediated disorder. The term “patient”, as used herein, means an animal, preferably a mammal, more preferably a human. Preferred pharmaceutical compositions of the invention are those formulated for oral, intravenous, or subcutaneous administration. However, any of the above dosage forms containing a therapeutically effective amount of a compound of the invention are well within the bounds of routine experimentation and therefore, well within the scope of the instant invention. In some embodiments, the pharmaceutical composition of the invention may further comprise another therapeutic agent. In some embodiments, such other therapeutic agent is one that is normally administered to patients with the disease or condition being treated.

By “therapeutically effective amount” is meant an amount sufficient to cause a detectable decrease in protein kinase activity or the severity of a Raf kinase-mediated disorder. The amount of Raf kinase inhibitor needed will depend on the effectiveness of the inhibitor for the given cell type and the length of time required to treat the disorder. It should also be understood that a specific dosage and treatment regimen for any particular patient will depend upon a variety of factors, including the activity of the specific compound employed, the age, body weight, general health, sex, and diet of the patient, time of administration, rate of excretion, drug combinations, the judgment of the treating physician, and the severity of the particular disease being treated. The amount of additional therapeutic agent present in a composition of this invention typically will be no more than the amount that would normally be administered in a composition comprising that therapeutic agent as the only active agent. Preferably, the amount of additional therapeutic agent will range from about 50% to about 100% of the amount normally present in a composition comprising that agent as the only therapeutically active agent.

In another aspect, the invention provides a method for treating a patient having, or at risk of developing or experiencing a recurrence of, a Raf kinase-mediated disorder. As used herein, the term “Raf kinase-mediated disorder” includes any disorder, disease or condition which is caused or characterized by an increase in Raf kinase expression or activity, or which requires Raf kinase activity. The term “Raf kinase-mediated disorder” also includes any disorder, disease or condition in which inhibition of Raf kinase activity is beneficial.

The Raf kinase inhibitors of the invention can be used to achieve a beneficial therapeutic or prophylactic effect, for example, in subjects with a proliferative disorder. Non-limiting examples of proliferative disorders include chronic inflammatory proliferative disorders, e.g., psoriasis and rheumatoid arthritis; proliferative ocular disorders, e.g., diabetic retinopathy; benign proliferative disorders, e.g., hemangiomas; and cancer. As used herein, the term “cancer” refers to a cellular disorder characterized by uncontrolled or disregulated cell proliferation, decreased cellular differentiation, inappropriate ability to invade surrounding tissue, and/or ability to establish new growth at ectopic sites. The term “cancer” includes, but is not limited to, solid tumors and bloodborne tumors. The term “cancer” encompasses diseases of skin, tissues, organs, bone, cartilage, blood, and vessels. The term “cancer” further encompasses primary and metastatic cancers.

Non-limiting examples of solid tumors that can be treated with the disclosed Raf kinase inhibitors include pancreatic cancer; bladder cancer; colorectal cancer; breast cancer, including metastatic breast cancer; prostate cancer, including androgen-dependent and androgen-independent prostate cancer; renal cancer, including, e.g., metastatic renal cell carcinoma; hepatocellular cancer; lung cancer, including, e.g., non-small cell lung cancer (NSCLC), bronchioloalveolar carcinoma (BAC), and adenocarcinoma of the lung; ovarian cancer, including, e.g., progressive epithelial or primary peritoneal cancer; cervical cancer; gastric cancer; esophageal cancer; head and neck cancer, including, e.g., squamous cell carcinoma of the head and neck; skin cancer, including e.g., malignant melanoma; neuroendocrine cancer, including metastatic neuroendocrine tumors; brain tumors, including, e.g., glioma, anaplastic oligodendroglioma, adult glioblastoma multiforme, and adult anaplastic astrocytoma; bone cancer; soft tissue sarcoma; and thyroid carcinoma.

Non-limiting examples of hematologic malignancies that can be treated with the disclosed Raf kinase inhibitors include acute myeloid leukemia (AML); chronic myelogenous leukemia (CML), including accelerated CML and CML blast phase (CML-BP); acute lymphoblastic leukemia (ALL); chronic lymphocytic leukemia (CLL); Hodgkin's disease (HD); non-Hodgkin's lymphoma (NHL), including follicular lymphoma and mantle cell lymphoma; B-cell lymphoma; T-cell lymphoma; multiple myeloma (MM); Waldenstrom's macroglobulinemia; myelodysplastic syndromes (MDS), including refractory anemia (RA), refractory anemia with ringed siderblasts (RARS), (refractory anemia with excess blasts (RAEB), and RAEB in transformation (RAEB-T); and myeloproliferative syndromes.

The compounds of formula (I) are particularly useful in the treatment of cancers or cell types characterized by aberrant activation of the Ras-Raf-MEK-ERK pathway, including, without limitation, those characterized by an activating Ras and/or Raf mutation. In some embodiments, the compound or composition of the invention is used to treat a patient having or at risk of developing or experiencing a recurrence in a cancer selected from the group consisting of melanoma, colon, lung, breast, ovarian, sarcoma and thyroid cancer. In certain embodiments, the cancer is a melanoma.

In some embodiments, the Raf kinase inhibitor of the invention is administered in conjunction with another therapeutic agent. In some embodiments, the other therapeutic agent is one that is normally administered to patients with the disease or condition being treated. The Raf kinase inhibitor of the invention may be administered with the other therapeutic agent in a single dosage form or as a separate dosage form. When administered as a separate dosage form, the other therapeutic agent may be administered prior to, at the same time as, or following administration of the protein kinase inhibitor of the invention.

In some embodiments, a Raf kinase inhibitor of formula (I) is administered in conjunction with an anticancer agent. As used herein, the term “anticancer agent” refers to any agent that is administered to a subject with cancer for purposes of treating the cancer. Nonlimiting examples anticancer agents include: radiotherapy; immunotherapy; DNA damaging chemotherapeutic agents; and chemotherapeutic agents that disrupt cell replication.

Non-limiting examples of DNA damaging chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea).

Chemotherapeutic agents that disrupt cell replication include: paclitaxel, docetaxel, and related analogs; vincristine, vinblastin, and related analogs; thalidomide and related analogs (e.g., CC-5013 and CC-4047); protein tyrosine kinase inhibitors (e.g., imatinib mesylate and gefitinib); proteasome inhibitors (e.g., bortezomib); NF-κB inhibitors, including inhibitors of IκB kinase; antibodies which bind to proteins overexpressed in cancers and thereby downregulate cell replication (e.g., trastuzumab, rituximab, cetuximab, and bevacizumab); and other inhibitors of proteins or enzymes known to be upregulated, over-expressed or activated in cancers, the inhibition of which downregulates cell replication.

In order that this invention be more fully understood, the following preparative and testing examples are set forth. These examples illustrate how to make or test specific compounds, and are not to be construed as limiting the scope of the invention in any way.

### EXAMPLES

### Examples

### Definitions

AcOH acetic acid
ATP adenosine triphosphate
BCA bicinchoninic acid
BSA bovine serum albumin
BOC tert-butoxycarbonyl
DCC N,N′-dicyclohexyl carbodiimide
DCM dichloromethane
DIPEA diisopropyl ethyl amine
DMAP N,N-dimethylaminopyridine
DMEM Dulbecco's Modified Eagle's Medium
DMF N,N-dimethylformamide
DTT dithiothreitol
EDTA ethylenediaminetetraacetic acid
EtOAc ethyl acetate
FA formic acid
FBS fetal bovine serum
h hours
HEPES N-(2-Hydroxyethyl)piperazine-N′-(2-ethanesulfonic acid)
HPLC high performance liquid chromatography
HRMS high resolution mass spectrum
LCMS liquid chromatography mass spectrum
Me methyl
MeOH methanol
min minutes
MS mass spectrum
MTT methylthiazoletetrazolium
PBS phosphate buffered saline
PKA cAMP-dependent protein kinase
rt room temperature
TEA triethylamine
TFA trifluoroacetic acid
THF tetrahydrofuran
TMB 3,3′,5,5′-Tetramethylbenzidine
WST (4-[3-(4-iodophenyl)-2-(4-nitrophenyl)-2-H-5-tetrazolio]-1,3-benzene disulfonate sodium salt)
   Analytical LC-MS Methods
   LCMS Conditions

Spectra were run on a Phenominex Luna 5 μm C18 50 x 4.6 mm column on a Hewlett-Packard HP1100 using the following gradients:
Method Formic Acid (FA): Acetonitrile containing zero to 100 percent 0.1% formic acid in water (2.5 ml/min for a 3 minute run).
Method Ammonium Acetate (AA): Acetonitrile containing zero to 100 percent 10 mM ammonium acetate in water (2.5 ml/min for a 3 minute run).

### Example 1

### Preparation of Intermediates and Reagents 4-chloro-N-methylpyridine-2-carboxamide

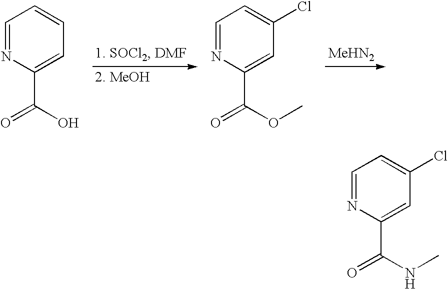

### Step 1: Preparation of methyl 4-chloropyridine-2-carboxylate

Anhydrous DMF (3 mL) was slowly added to thionyl chloride (90 mL) at 40° C. under nitrogen. The solution was stirred at 40° C. for 10 min, and pyridine 2-carboxylic acid (30.0 g, 243.7 mmol) was added portionwise over 10 min. The solution was heated at 72° C. for 16 h (a yellow precipitate formed). The mixture was cooled to rt, diluted with toluene (100 mL), and concentrated to small volume. This process was repeated two additional times before the mixture was concentrated to dryness. The dry yellow mixture was then cooled to 0° C., and methanol (200 mL) added dropwise via addition funnel. The mixture was stirred for 45 min and a thick white precipitate formed. Diethyl ether was added to the mixture and the white solid was filtered. Methyl 4-chloropyridine-2-carboxylate was collected in two crops (37.8 g, 91%). ¹H NMR (300 MHz, d₆-DMSO) δ: 10.00 (bs, 1H), 8.68 (d, 1H), 8.08 (d, 1H), 7.82 (dd, 1H), and 3.88 (s, 3H).

### Step 2: Preparation of 4-chloro-N-methylpyridine-2-carboxamide

To a solution of methyl 4-chloropyridine-2-carboxylate (29.9 g, 174.9 mmol) in MeOH (15 mL) at 0° C. was added 2M methylamine in THF (437 mL, 874 mmol) dropwise. The reaction mixture was allowed to stir at 0° C. for 3 h. The mixture was then concentrated and extracted with EtOAc (2×). The organic solutions were combined, washed with brine, dried over Na₂SO₄, filtered, and concentrated to yield 4-chloro-N-methylpyridine-2-carboxamide (25 g, 84%). ¹H NMR (300 MHz, d₆-DMSO) δ: 8.85 (bs, 1H), 8.61 (d, 1H), 8.00 (d, 1H), 7.74 (dd, 1H), 2.81 (d, 3H).

### 4-chloro-2-(4,5-dihydro-1H-imidazol-2-yl)pyridine

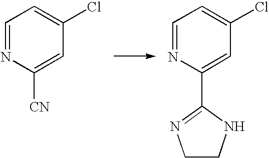

To a solution of 4-chloropyridine-2-carbonitrile (20.0 g, 121 mmol, prepared as described by Sakamoto et al. Chem. Pharm. Bull. 1985, 33, 565-571) in MeOH (240 mL), was added sodium methoxide (0.655 g, 12.1 mmol). The reaction mixture was stirred at rt under an atmosphere of argon for 2 h. Ethylene diamine (40.0 mL, 597 mmol) was added to the reaction mixture was stirred at 50° C. for 20 h. The solution was allowed to cool to rt and concentrated. The residue was partitioned between water and DCM. The organic solution was separated, dried over MgSO₄, filtered and concentrated to give the desired product as a light brown solid (21.9 g, >99%). LCMS: (FA) ES+182.1 (M+1).

### 3-{2-[(tert-butoxycarbonyl)amino]-1-methylethyl}benzoic acid

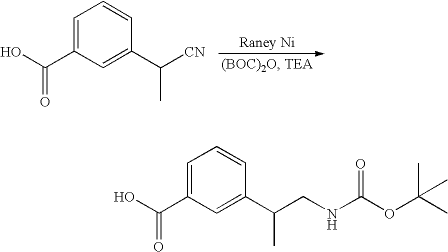

To a solution of 3-(1-cyanoethyl)benzoic acid (1.0 g, 5.7 mmol) in THF (50 mL) were added TEA (3.96 mL, 28.6 mmol) and (BOC)₂O (3.7 g, 17.1 mmol). The solution was degassed with nitrogen and then Raney Ni was added. The mixture was degassed with hydrogen and stirred at rt overnight. The reaction mixture was filtered through celite and concentrated. The residue was redissolved in DCM and washed with 1N HCl. The organic solution was washed with brine, dried over Na₂SO₄, filtered, and concentrated. The residue was purified by column chromatography to give recovered 3-(1-cyanoethyl)benzoic acid (198 mg) and 3-{2-[(tert-butoxycarbonyl)amino]-1-methylethyl}benzoic acid (911 mg, 57% (72% based on recovered starting material)) as a white solid. ¹H NMR (300 MHz, CD₃OD) δ: 7.82-7.91 (m, 2H), 7.35-7.50 (m, 2H), 3.15-3.22 (m, 2H), 2.90-3.05 (m, 1H), 1.37 (s, 9H), and 1.25 (d, 3H).

### Polymeric 4-[(aminocarbonyloxy)-2,3,5,6-tetrafluorophenyl 3-(trifluoromethyl)benzoate

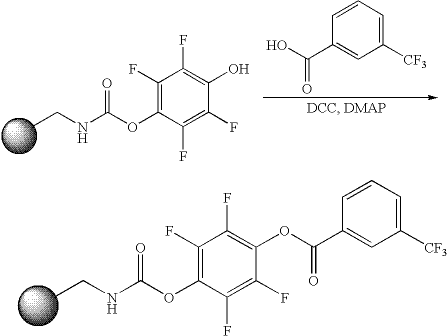

To pre swelled TFP resin [(Polymerlabs, Cat. No. 3474-1689), 100 mg, 0.131 mmol] in DMF (1 mL) was added 3-(trifluoromethyl)benzoic acid (0.26 mmol) in DMF (0.5 mL). The mixture was agitated for five min and then DMAP (12 mg, 0.098 mmol) and DCC (54 mg, 0.26 mmol) were added. The reaction mixture was agitated for 48 hr. The resin was filtered and washed with DMF (3×5 mL), THF (3×5 mL), DCM (3×5 mL), and Et₂O (2×5 ml) and then dried to yield polymeric 4-[(aminocarbonyl)oxy]-2,3,5,6-tetrafluorophenyl 3-(trifluoromethyl)benzoate.

### Example 2

### Synthesis of N-methyl-4-[3-(2-{[3-(trifluoromethyl)benzoyl]amino}ethyl)-phenoxy]pyridine-2-carboxamide (I-192)

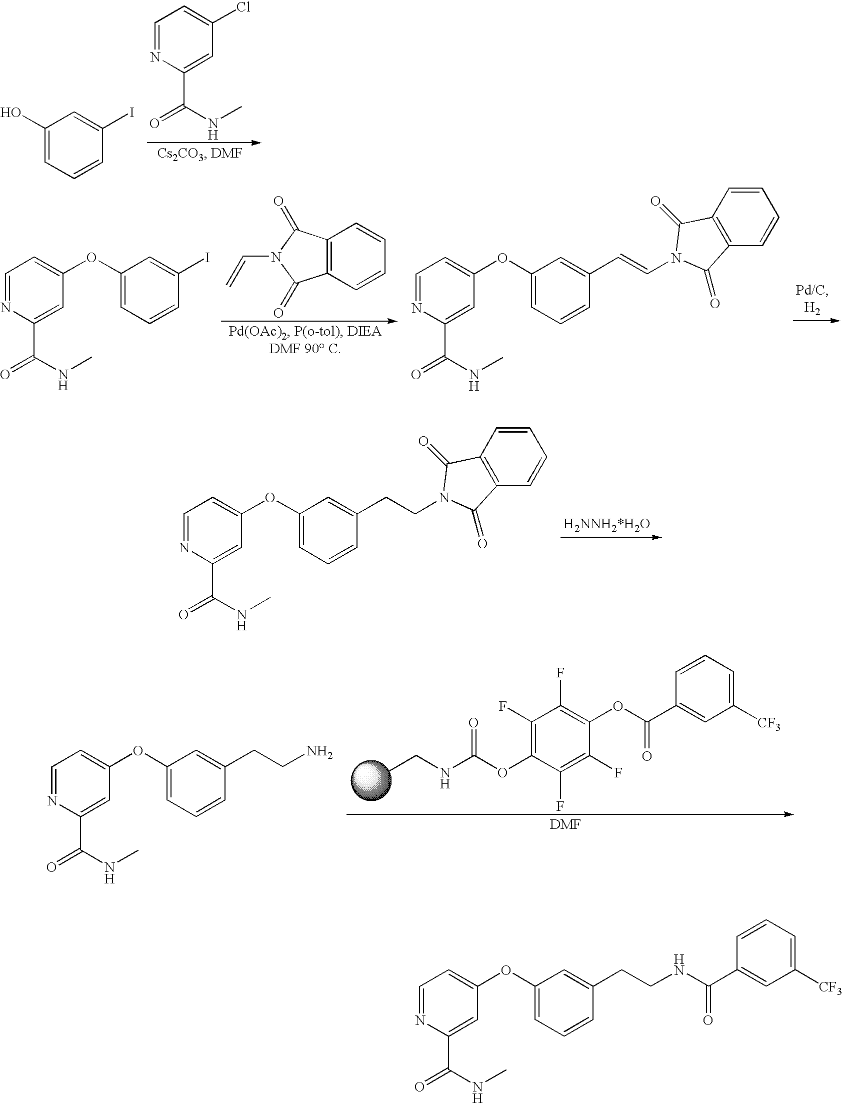

### Step 1: Preparation of 4-(3-iodophenoxy)-N-methylpyridine-2-carboxamide

To a solution of 3-iodophenol (6.20 g, 28.2 mmol) in anhydrous DMF was added Cs₂CO₃ (27.5 g, 84.5 mmol) and 4-chloro-N-methylpyridine-2-carboxamide (5.74 g, 33.8 mmol). The reaction mixture was heated at 100° C. overnight. The reaction was then cooled to rt and concentrated. Water (200 mL) was added to the mixture. A light brown precipitate formed and was filtered to give 4-(3-iodophenoxy)-N-methylpyridine-2-carboxamide in quantitative yield. ¹H NMR (300 MHz, d₆-DMSO) δ: 8.23-8.76 (m, 1H), 8.52 (d, 1H), 7.72 (d, 1H), 7.64 (t, 1H), 7.38 (d, 1H), 7.25-7.32 (m, 2H), 7.16-7.17 (m, 1H), and 2.78 (d, 3H).

### Step 2: Preparation of 4-{3-[(E)-2-(1,3-dioxo-1,3-dihydro-2H-isoindol-2-yl)vinyl]-phenoxy}-N-methylpyridine-2-carboxamide

To a degassed solution of solution 4-(3-iodophenoxy)-N-methylpyridine-2-carboxamide (11.4 g, 32.4 mmol) in anhydrous DMF (100 mL) was added palladium acetate (0.15 g, 0.65 mmol), tri-o-tolylphosphine (0.79 g, 2.58 mmol), 2-vinyl-1H-isoindole-1,3(2H)-dione (5.60 g, 32.4 mmol), and DIPEA (11.5 mL 64.5 mmol). After degassing the mixture again, the reaction was heated at 90° C. overnight under nitrogen. The reaction mixture was then cooled to rt and concentrated. The mixture was diluted with water and extracted with DCM (2×). The organic solutions were combined and washed with brine, dried over Na₂SO₄, filtered, and concentrated. The residue was purified by column chromatography to give 4-{3-[(E)-2-(1,3-dioxo-1,3-dihydro-2H-isoindol-2-yl)vinyl]-phenoxy)-N-methylpyridine-2-carboxamide (8.9 g, 69%) as a yellow solid. ¹H NMR (300 MHz, CDCl₃) δ: 8.40 (d, 1H), 7.96-8.05 (m, 1H), 7.89-7.94 (m, 2H), 7.73-7.80 (m, 3H), 7.65 (d, 1H), 7.33-7.44 (m, 3H), 7.20 (t, 1H), 6.96-7.01 (m, 2H), and 3.01 (d, 3H).

### Step 3: Preparation of 4-{3-[2-(1,3-dioxo-1,3-dihydro-2H-isoindol-2-yl)ethyl]phenoxy}-N-methylpyridine-2-carboxamide

To a solution of 4-{3-[(E)-2-(1,3-dioxo-1,3-dihydro-2H-isoindol-2-yl)vinyl]-phenoxy}-N-methylpyridine-2-carboxamide (6.0 g, 15.0 mmol) in ETOH (42 mL) and THF (30 mL) was added 10% palladium on charcoal (600 mg). The reaction mixture was stirred under hydrogen at 50 psi for two days. The mixture was carefully filtered through celite, and rinsed with DCM (500 mL). The solvent was evaporated to give 4-{3-[2-(1,3-dioxo-1,3-dihydro-2H-isoindol-2-yl)ethyl]phenoxy}-N-methylpyridine-2-carboxamide (5.85 g, 97%). ¹H NMR (300 MHz, CDCl₃) δ: 8.28 (d, 1H), 8.03 (bd, 1H), 7.74-7.81 (m, 2H), 7.62-7.69 (m, 2H), 7.27 (t, 1H), 1.08 (d, 1H), 6.95 (t, 1H), 6.87-6.92 (m, 1H), 6.82-6.85 (m, 1H), 3.89 (t, 2H), and 2.94-3.01 (m, 5H).

### Step 4: Preparation of 4-[3-(2-aminoethyl)phenoxy]-N-methylpyridine-2-carboxamide

To a mixture of 4-{3-[2-(1,3-dioxo-1,3-dihydro-2H-isoindol-2-yl)ethyl]phenoxy]-N-methylpyridine-2-carboxamide (5.85 g, 14.5 mmol) in EtOH (50 ml) was added hydrazine hydrate (5 mL). The mixture was heated at 80° C. for 3 h and a white precipitate formed. The solid was filtered off and washed with EtOH (500 mL). The organic solutions were concentrated and the residual solid was filtered off in the same manner (2×). The oil residue was purified by column chromatography to give 4-[3-(2-aminoethyl)phenoxy]-N-methylpyridine-2-carboxamide (3.32 g, 84%). ¹H NMR (300 MHz, CDCl₃) δ: 8.51 (d, 1H), 8.28 (bd, 1H), 7.84 (d, 1H), 7.46-7.52 (m, 1H), 7.23-7.26 (m, 1H), 7.07-7.12 (m, 3H), 3.09-3.15 (m, 5H), 2.90 (t, 2H), and 2.04 (bd, 2H).

### Step 5: Preparation of N-methyl-4-[3-(2-{[3-(trifluoromethyl)benzoyl]amino}ethyl)-phenoxy]pyridine-2-carboxamide (I-192)

To pre swelled polymeric 4-[(aminocarbonyl)oxy]-2,3,5,6-tetrafluorophenyl 3-(trifluoromethyl)benzoate in DCM (1 mL) was added 4-[3-(2-aminoethyl)phenoxy]-N-methylpyridine-2-carboxamide (32 mg, 0.12 mmol) in DMF (1 mL). The mixture was agitated for 24 hr and then the resin was filtered and washed with DCM (3×2 mL). The combined organic solutions were concentrated and purified by Agilent reverse phase HPLC to yield N-methyl-4-[3-(2-{[3-(trifluoromethyl)benzoyl]amino}ethyl)-phenoxy]pyridine-2-carboxamide26.7 mg, 51%). LCMS: (AA) ES+443.4 (M+1). ¹H NMR (400 MHz, d₆-DMSO) δ: 8.80-8.85 (m, 1H), 8.71-8.77 (m 1H), 8.44 (d, 1H), 8.13 (s, 1H), 8.10 (d, 1H), 7.88 (d, 1H), 7.69 (t, 1H), 7.39-7.47 (m, 2H), 7.23 (d, 1H), 7.04-7.13 (m, 3H), 3.51-3.59 (m, 2H), 2.89-2.96 (m, 2H), and 2.79 (d, 3H).

Compounds in the following table were prepared from the appropriate starting materials in a method analogous to that of Example 2:

| | |
|---|---|
| I-56 | LCMS: (AA) ES+ 419.5 (M + 1). |
| I-72 | LCMS: (AA) ES+ 465.9 (M + 1). |
| I-24 | LCMS: (AA) ES+ 459.4 (M + 1). |
| I-30 | LCMS: (AA) ES+ 429.4 (M + 1). |
| I-25 | LCMS: (AA) ES+ 435.8 (M + 1). |
| I-23 | LCMS: (AA) ES+ 406.4 (M + 1). |
| I-120 | LCMS: (AA) ES+ 407.5 (M + 1). |
| I-115 | LCMS: (AA) ES+ 427.9 (M + 1). |
| I-121 | LCMS: (AA) ES+ 461.4 (M + 1). |
| I-40 | LCMS: (AA) ES+ 423.5 (M + 1). |
| I-104 | LCMS: (AA) ES+ 468.4 (M + 1). |
| I-76 | LCMS: (AA) ES+ 417.5 (M + 1). |
| I-102 | LCMS: (AA) ES+ 444.3 (M + 1). |
| I-74 | LCMS: (AA) ES+ 444.3 (M + 1). |
| I-66 | LCMS: (AA) ES+ 391.0 (M + 1). |
| I-19 | LCMS: (AA) ES+ 405.5 (M + 1). |
| I-10 | LCMS: (AA) ES+ 541.6 (M + 1). |
| I-94 | LCMS: (AA) ES+ 405.5 (M + 1). |
| I-89 | LCMS: (AA) ES+ 389.5 (M + 1). |
| I-131 | LCMS: (AA) ES+ 439.5 (M + 1). |
| I-37 | LCMS: (AA) ES+ 469.9 (M + 1). |
| I-106 | LCMS: (AA) ES+ 417.5 (M + 1). |
| I-109 | LCMS: (AA) ES+ 451.5 (M + 1). |
| I-124 | LCMS: (AA) ES+ 445.9 (M + 1). |
| I-52 | LCMS: (AA) ES+ 421.5 (M + 1). |
| I-95 | LCMS: (AA) ES+ 395.5 (M + 1). |
| I-108 | LCMS: (AA) ES+ 393.4 (M + 1). |
| I-127 | LCMS: (AA) ES+ 378.4 (M + 1). |
| I-78 | LCMS: (AA) ES+ 405.5 (M + 1). |
| I-35 | LCMS: (AA) ES+ 427.9 (M + 1). |
| I-69 | LCMS: (AA) ES+ 406.4 (M + 1). |
| I-118 | LCMS: (AA) ES+ 465.5 (M + 1). |
| I-43 | LCMS: (AA) ES+ 467.5 (M + 1). |
| I-9 | LCMS: (AA) ES+ 435.5 (M + 1). |
| I-132 | LCMS: (AA) ES+ 461.4 (M + 1). |
| I-71 | LCMS: (AA) ES+ 468.4 (M + 1). |
| I-65 | LCMS: (AA) ES+ 463.5 (M + 1). |
| I-22 | LCMS: (AA) ES+ 454.5 (M + 1). |
| I-112 | LCMS: (AA) ES+ 433.5 (M + 1). |
| I-53 | LCMS: (AA) ES+ 439.5 (M + 1). |
| I-85 | LCMS: (AA) ES+ 403.5 (M + 1). |
| I-96 | LCMS: (AA) ES+ 403.5 (M + 1). |
| I-47 | LCMS: (AA) ES+ 411.4 (M + 1). |
| I-75 | LCMS: (AA) ES+ 461.4 (M + 1). |
| I-32 | LCMS: (AA) ES+ 454.3 (M + 1). |
| I-114 | LCMS: (AA) ES+ 465.5 (M + 1). |
| I-119 | LCMS: (AA) ES+ 462.3 (M + 1). |
| I-14 | LCMS: (AA) ES+ 439.9 (M + 1). |
| I-49 | LCMS: (AA) ES+ 468.4 (M + 1). |
| I-34 | LCMS: (AA) ES+ 400.4 (M + 1). |
| I-67 | LCMS: (AA) ES+ 435.5 (M + 1). |
| I-87 | LCMS: (AA) ES+ 411.4 (M + 1). |
| I-13 | LCMS: (AA) ES+ 495.9 (M + 1). |
| I-91 | LCMS: (AA) ES+ 414.5 (M + 1). |
| I-100 | LCMS: (AA) ES+ 474.4 (M + 1). |
| I-103 | LCMS: (AA) ES+ 435.5 (M + 1). |
| I-194 | LCMS: (AA) ES+ 443.5 (M + 1). |
| I-79 | LCMS: (AA) ES+ 440.5 (M + 1). |
| I-15 | LCMS: (AA) ES+ 439.9 (M + 1). |
| I-86 | LCMS: (AA) ES+ 467.5 (M + 1). |
| I-64 | LCMS: (AA) ES+ 439.9 (M + 1). |
| I-27 | LCMS: (AA) ES+ 478.9 (M + 1). |
| I-54 | LCMS: (AA) ES+ 445.9 (M + 1). |
| I-133 | LCMS: (AA) ES+ 407.5 (M + 1). |
| I-88 | LCMS: (AA) ES+ 396.5 (M + 1). |
| I-116 | LCMS: (AA) ES+ 409.9 (M + 1). |
| I-6 | LCMS: (AA) ES+ 490.4 (M + 1). |
| I-41 | LCMS: (AA) ES+ 534.1 (M + 1). |
| I-111 | LCMS: (AA) ES+ 467.2 (M + 1). |
| I-62 | LCMS: (AA) ES+ 433.1 (M + 1). |
| I-3 | LCMS: (AA) ES+ 481.1 (M + 1). |
| I-33 | LCMS: (AA) ES+ 473.2 (M + 1). |
| I-98 | LCMS: (AA) ES+ 528.2 (M + 1). |
| I-130 | LCMS: (AA) ES+ 443.2 (M + 1). |
| I-58 | LCMS: (AA) ES+ 433.1 (M + 1). |
| I-83 | LCMS: (AA) ES+ 458.1 (M + 1). |
| I-77 | LCMS: (AA) ES+ 472.2 (M + 1). |
| I-31 | LCMS: (AA) ES+ 508.2 (M + 1). |
| I-20 | LCMS: (AA) ES+ 450.2 (M + 1). |
| I-39 | LCMS: (AA) ES+ 407.2 (M + 1). |
| I-122 | LCMS: (AA) ES+ 532.2 (M + 1). |
| I-28 | LCMS: (AA) ES+ 486.2 (M + 1). |
| I-99 | LCMS: (AA) ES+ 483.2 (M + 1). |
| I-38 | LCMS: (AA) ES+ 554.2 (M + 1). |
| I-84 | LCMS: (AA) ES+ 526.2 (M + 1). |
| I-73 | LCMS: (AA) ES+ 418.2 (M + 1). |
| I-11 | LCMS: (AA) ES+ 441.2 (M + 1). |
| I-125 | LCMS: (AA) ES+ 458.1 (M + 1). |
| I-5 | LCMS: (AA) ES+ 512.2 (M + 1). |
| I-60 | LCMS: (AA) ES+ 446.2 (M + 1). |
| I-17 | LCMS: (AA) ES+ 468.1 (M + 1). |
| I-16 | LCMS: (AA) ES+ 426.2 (M + 1). |
| I-105 | LCMS: (AA) ES+ 457.1 (M + 1). |
| I-29 | LCMS: (AA) ES+ 447.1 (M + 1). |
| I-70 | LCMS: (AA) ES+ 437.2 (M + 1). |
| I-36 | LCMS: (AA) ES+ 485.2 (M + 1). |
| I-81 | LCMS: (AA) ES+ 487.0 (M + 1). |
| I-117 | LCMS: (FA) ES+ 446.1 (M + 1). |
| I-59 | LCMS: (FA) ES+ 420.2 (M + 1). |
| I-18 | LCMS: (FA) ES+ 420.1 (M + 1). |
| I-55 | LCMS: (FA) ES+ 496.1 (M + 1). |
| I-101 | LCMS: (FA) ES+ 420.1 (M + 1). |
| I-110 | LCMS: (FA) ES+ 502.0 (M + 1). |
| I-46 | LCMS: (FA) ES+ 520.1 (M + 1). |
| I-44 | LCMS: (FA) ES+ 462.1 (M + 1). |
| I-93 | LCMS: (FA) ES+ 412.1 (M + 1). |
| I-82 | LCMS: (FA) ES+ 504.1 (M + 1). |
| I-8 | LCMS: (FA) ES+ 444.1 (M + 1). |
| I-4 | ¹H NMR (400 MHz, CD₃OD) δ: 8.72-8.79 (m, 1H), 8.52-8.58 (m, 1H), 8.43 (d, 1H), |
| | 7.39-7.46 (m, 2H), 7.18-7.23 (m, 1H), 7.13-7.17 (m, 1H), 7.02-7.11 (m, 3H), |
| | 6.85-6.98 (m, 2H), 3.67-3.75 (m, 4H), 3.46-3.54 (m, 2H), 3.12-3.18 (m, 4H), 2.84-2.92 (m, |
| | 2H), and 2.77 (d, 3H). |
| I-42 | ¹H NMR (300 MHz, CD₃OD) δ: 8.34 (d, 1H), 7.52-7.65 (m, 3H), 7.29-7.45 (m, 3H), |
| | 7.20 (d, 1H), 6.94-7.03 (m, 3H), 3.60-3.66 (m, 2H), 3.15-3.23 (m, 2H), 2.93 (s, 3H), |
| | 2.89-3.01 (m, 3H), 1.37 (s, 9H), and 1.23 (d, 3H). |
| I-7 | (after deprotection of I-42 with TFA/DCM) ¹H NMR (300 MHz, CD₃OD) δ: |
| | 8.35 (d, 1H), 7.51-7.64 (m, 3H), 7.31-7.45 (m, 3H), 7.20 (d, 1H), 6.94-7.01 (m, 3H), |
| | 3.59-3.67 (m, 2H), 2.94-3.01 (m, 2H), 2.92 (s, 3H), 2.74-2.84 (m, 3H), and 1.24 (d, 3H). |

### Example 3

### Synthesis of 4-[3-(2-([4-chloro-3-(trifluoromethyl)benzoylamino]ethyl)phenoxy]-N-methylpyridine-2-carboxamide (I-12)

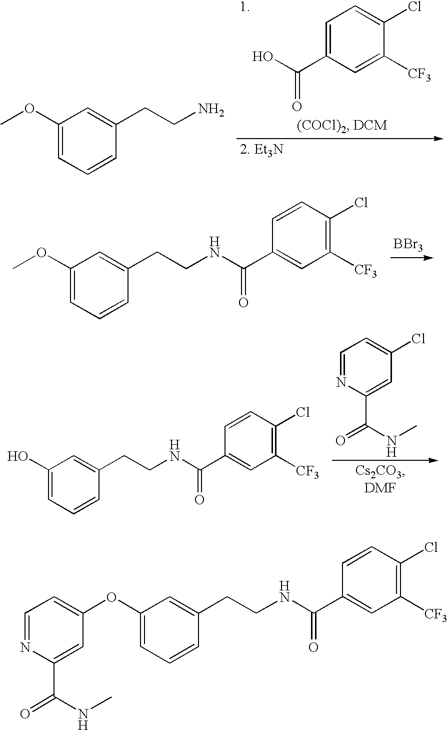

### Step 1: Preparation of 4-chloro-N-[2-(3-methoxyphenyl)ethyl]-3-(trifluoromethyl)-benzamide

To a solution of 4-chloro-3-(trifluoromethyl)benzoic acid (2.0 g, 8.9 mmol) in DCM was added oxalyl chloride (1.55 mL, 17.8 mmol) dropwise. To this solution was added a few drops of DMF. The reaction mixture was allowed to stir for 1 h and then concentrated. The residue was redissolved in DCM and to this solution were added 2-(3-methoxyphenyl)ethanamine (1.43 mL, 9.8 mmol) and TEA (2.48 mL, 17.8 mmol). The reaction mixture was allowed to stir at rt overnight. The reaction was quenched by the addition of 1N HCl and then the solutions were separated. The organic solution was washed with brine, dried over Na₂SO₄, filtered, and concentrated to give 4-chloro-N-[2-(3-methoxyphenyl)ethyl]-3-(trifluoromethyl)-benzamide (3.32 g) which was used without further purification.

### Step 2: Preparation of 4-chloro-N-[2-(3-hydroxyphenyl)ethyl]-3-(trifluoromethyl)-benzamide

To a solution of 4-chloro-N-[2-(3-methoxyphenyl)ethyl]-3-(trifluoromethyl)-benzamide (1.33 g, 3.87 mmol) in DCM (25 mL) was added BBr₃ (1M in DCM, 7.73 mL) at 0° C. The solution was allowed to warm to rt. After 1 h, the reaction mixture was poured onto ice and neutralized with conc. NH₄OH. The precipitate that formed was rinsed with Et₂O and dissolved in EtOAc. The organic solution was washed with water and brine, dried over Na₂SO₄, filtered, and concentrated to give 4-chloro-N-[2-(3-hydroxyphenyl)ethyl]-3-(trifluoromethyl)-benzamide (539 mg) as a white solid which was used without further purification.

### Step 3: Preparation of 4-[3-(2-{[4-chloro-3-(trifluoromethyl)benzoyl]amino}-ethyl)phenoxy]-N-methylpyridine-2-carboxamide (I-12)

A slurry of 4-chloro-N-[2-(3-hydroxyphenyl)ethyl]-3-(trifluoromethyl)-benzamide (0.64 g, 1.9 mmol), Cs₂CO₃ (1.83 g, 5.6 mmol) and 4-chloro-N-methylpyridine-2-carboxamide (0.38 g, 2.2 mmol) in DMF (4 mL) was heated at 100° C. overnight. The reaction mixture was diluted with EtOAc, washed with water and brine, dried over Na₂SO₄, filtered, and concentrated. The residue was purified by column chromatography to give 4-[3-(2-{[4-chloro-3-(trifluoromethyl)benzoyl]amino}-ethyl)phenoxy]-N-methylpyridine-2-carboxamide (I-12). ¹H NMR (400 MHz, d₆-DMSO) δ: 8.85-8.92 (m, 1H), 8.71-8.79 (m 1H), 8.45 (d, 1H), 8.21 (s, 1H), 8.05-8.10 (m, 1H), 7.82 (d, 1H), 7.38-7.46 (m, 2H), 7.22 (d, 1H), 7.04-7.13 (m, 3H), 3.51-3.58 (m, 2H), 2.88-2.94 (m, 2H), and 2.78 (d, 3H).

Compounds in the following table were prepared from the appropriate starting materials in a method analogous to that of Example 3:

| | |
|---|---|
| I-51 | ¹H NMR (300 MHz, d₆-DMSO) δ: 8.73-8.80 (m, 1H), 8.40-8.45 (m, 2H), |
| | 7.39-7.46 (m, 2H), 7.15-7.24 (m, 2H), 7.01-7.11 (m, 4H), 6.79-6.86 (m, 1H), |
| | 3.44-3.53 (m, 2H), 2.84-2.93 (m, 8H), and 2.77 (d, 3H). |
| I-26 | ¹H NMR (300 MHz, d₆-DMSO) δ: 8.71-8.79 (m, 2H), 8.43 (d, 2H), 7.82 (dt, |
| | 1H), 7.72-7.75 (m, 1H), 7.48-7.61 (m, 2H), 7.39-7.46 (m, 2H), 7.19-7.24 (m, |
| | 1H), 7.02-7.12 (m, 3H), 3.47-3.57 (m, 2H), 2.86-2.94 (m, 2H), and 2.77 (d, |
| | 3H). |
| I-61 | ¹H NMR (300 MHz, d₆-DMSO) δ: 8.73-8.80 (m, 1H), 8.60-8.66 (m, 1H), |
| | 8.44 (d, 1H), 7.79 (d, 2H), 7.38-7.53 (m, 4H), 7.21 (d, 1H), 7.01-7.12 (m, 3H), |
| | 3.46-3.56 (m, 2H), 2.85-2.92 (m, 2H), and 2.78 (d, 3H). |
| I-193 | ¹H NMR (300 MHz, d₆-DMSO) δ: 8.73-8.80 (m, 1H), 8.45-8.52 (m, 1H), |
| | 8.42 (d, 1H), 7.53-70.61 (m, 2H), 7.39-7.46 (m, 2H), 7.27-7.32 (m, 2H), 7.21 (d, |
| | 1H), 7.02-7.11 (m, 3H), 3.45-3.54 (m, 2H), 2.85-2.92 (m, 2H), and 2.77 (d, |
| | 3H). LCMS: (AA) ES+ 389.6 (M + 1). |
| I-129 | ¹H NMR (300 MHz, CD₃OD) δ: 8.60 (d, 1H), 8.19-8.24 (m, 1H), |
| | 8.02-8.07 (m, 1H), 7.81-7.85 (m, 1H), 7.74 (d, 1H), 7.43 (d, 2H), 7.32-7.38 (m, 1H), |
| | 7.17 (d, 2H), 3.42-3.49 (m, 2H), 2.96 (s, 3H), and 2.76-2.83 (m, 2H). |
| I-68 | ¹H NMR (300 MHz, d₆-DMSO, HCl salt) δ: 8.90-8.97 (m, 1H), 8.76-8.84 (m, |
| | 1H), 8.46 (d, 1H), 8.20-8.24 (m, 1H), 8.06-8.12 (m, 1H), 7.82 (d, 1H), 7.40 (d, |
| | 1H), 7.34 (d, 2H), 7.08-7.15 (m, 3H), 3.46-3.55 (m, 2H), 2.82-2.92 (m, 2H), |
| | and 2.74 (d, 3H). |

### Example 4

### Synthesis of 4-chloro-N-[2-(3-{[2-(4,5-dihydro-1H-imidazol-2-yl)pyridin-4-yl]oxy}phenyl)ethyl]-3-(trifluoromethyl)benzamide (I-92)

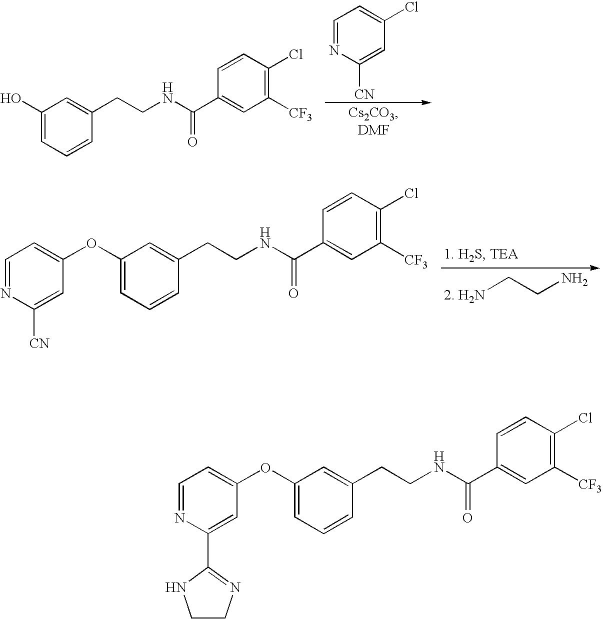

### Step 1: Preparation of 4-chloro-N-(2-{3-[(2-cyanopyridin-4-yl)oxy]phenyl}ethyl)-3-(trifluoromethyl)benzamide

To a solution of 4-chloropyridine-2-carbonitrile (1.1 g, 8.1 mmol) and 4-chloro-N-[2-(3-hydroxyphenyl)ethyl]-3-(trifluoromethyl)benzamide (3.0 g, 8.9 mmol) in DMF (100 mL) was added Cs₂CO₃ (7.9 g, 24.3 mmol). The reaction mixture was heated at 50° C. for 24 h and then cooled to rt and concentrated. The residue was diluted with EtOAc and 1N HCl was added. The organic solution was separated and further washed with 1N HCl and brine, dried over Na₂SO₄, filtered, and concentrated. The residue was purified by column chromatography to give 4-chloro-N-(2-{3-[(2-cyanopyridin-4-yl)oxy]phenyl}ethyl)-3-(trifluoromethyl)benzamide (2.6 g) as a yellow solid.

### Step 2: Preparation of 4-chloro-N-[2-(3-{[2-(4,5-dihydro-1H-imidazol-2-yl)pyridin-4-yl]oxy}phenyl)ethyl]-3-(trifluoromethyl)benzamide (I-92)

H₂S was bubbled through a solution of 4-chloro-N-(2-{3-[(2-cyanopyridin-4-yl)oxy]phenyl}ethyl)-3-(trifluoromethyl)benzamide (0.46 g, 1.0 mmol) and TEA (1.4 mL, 10.4 mmol) in EtOH (3 mL) for -3 min. The resulting yellow solution was stirred at rt for 20 min and then diluted with EtOAc and water. The organic solution was separated and further washed with water and brine, dried over Na₂SO₄, filtered, and concentrated. The resulting oil was dissolved in ethane-1,2-diamine (3 mL) and stirred at rt for 1.5 h. The reaction mixture was diluted with EtOAc and water. The organic solution was separated and further washed with water and brine, dried over Na₂SO₄, filtered, and concentrated to give 4-chloro-N-[2-(3-{[2-(4,5-dihydro-1H-imidazol-2-yl)pyridin-4-yl)oxy}phenyl)ethyl]-3-(trifluoromethyl)benzamide (I-92) as a pale yellow solid. The solid was dissolved in MeOH and treated with 1N HCl in Et₂O to provide the HCl salt of I-92. ¹H NMR (300 MHz, CD₃OD, HCl salt) δ: 8.59 (d, 1H), 8.10 (d, 1H), 7.93 (dd, 1H), 7.71 (d, 1H), 7.59 (d, 1H), 7.41 (t, 1H), 7.18-7.24 (m, 1H), 7.13-7.16 (m, 1H), 7.02 (dd, 1H), 4.09 (s, 4H), 3.69 (t, 2H), and 2.97 (t, 2H).

Compounds in the following table were prepared from the appropriate starting materials in a method analogous to that of Example 4:

| | |
|---|---|
| I-57 | ¹H NMR (300 MHz, d₆-DMSO) δ: 8.81-8.86 (m, 1H), 8.44 (d, 1H), 8.18 (s, 1H), |
| | 8.05-8.14 (m, 2H), 7.84-7.91 (m, 1H), 7.63-7.73 (m, 1H), 7.38-7.46 (m, 2H), 7.19-7.23 (m, |
| | 1H), 7.01-7.12 (m, 3H), 3.61 (s, 4H), 3.48-3.58 (m, 2H), and 2.87-2.94 (m, 2H). |
| I-126 | ¹H NMR (300 MHz, d₆-DMSO) δ: 8.72-8.78 (m, 1H), 8.44 (d, 1H), 8.19 (s, 1H), |
| | 7.79-7.85 (m, 1H), 7.73 (br s, 1H), 7.49-7.62 (m, 2H), 7.38-7.46 (m, 2H), 7.20 (d, 1H), |
| | 7.00-7.12 (m, 3H), 3.61 (s, 4H), 3.46-3.57 (m, 2H), and 2.84-2.93 (m, 2H). |
| I-1 | ¹H NMR (300 MHz, d₆-DMSO) δ: 8.58-8.66 (m, 1H), 8.45 (d, 1H), 8.18 (s, 1H), |
| | 7.79 (d, 2H), 7.50 (d, 2H), 7.39-7.45 (m, 2H), 7.18-7.22 (m, 1H), 7.01-7.10 (m, 2H), |
| | 3.61 (s, 4H), 3.45-3.56 (m, 2H), and 2.84-2.93 (m, 2H). |
| I-128 | ¹H NMR (300 MHz, d₆-DMSO, 2*HCl salt) δ: 10.86 (s, 2H), 8.86-8.93 (m, 1H), |
| | 8.68 (d, 1H), 8.25-8.28 (m, 1H), 8.12-8.18 (m, 1H), 7.92-7.96 (m, 1H), 7.85 (d, 1H), |
| | 7.39-7.48 (m, 1H), 7.19-7.26 (m, 2H), 7.10-7.14 (m, 1H), 7.01-7.08 (m, 1H), 3.99 (s, 4H), |
| | 3.24-3.35 (m, 2H), 2.64-2.73 (m, 2H), and 1.79-1.93 (m, 2H). |

### Example 5

### Synthesis of N-[2-[3-({2-[(4,5-dihydro-1H-imidazol-2-ylamino)methyl]pyridin-4-yl}oxy)phenyl]ethyl]-3-(trifluoromethyl)benzamide (I-2)

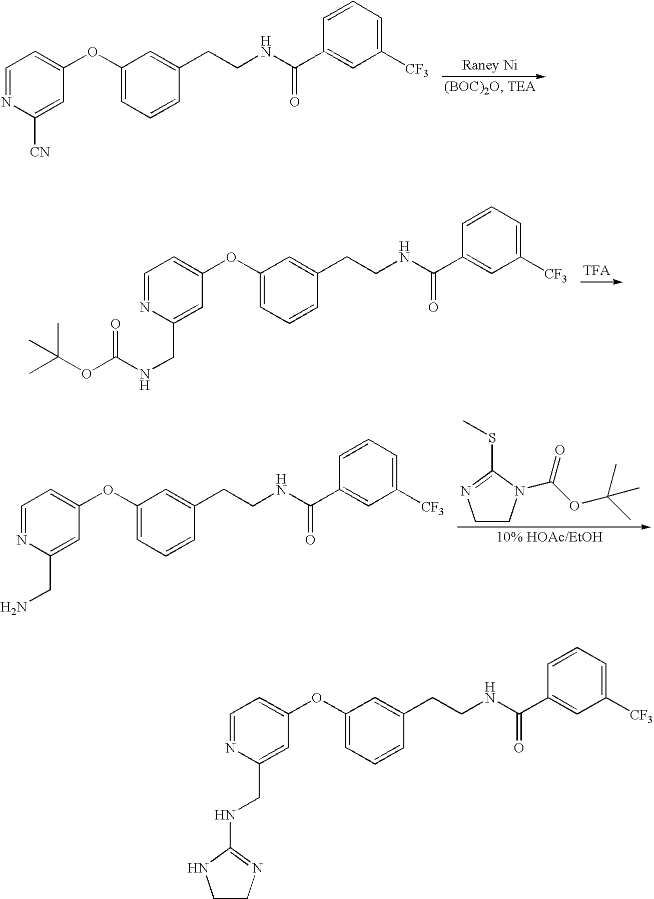

### Step 1: Preparation of tert-butyl({4-[3-(2-{[3-(trifluoromethyl)benzoyl]amino}ethyl)-phenoxy]pyridin-2-yl}methyl)carbamate (I-50)

To a solution of N-(2-{3-[(2-cyanopyridin-4-yl)oxy]phenyl}ethyl)-3-(trifluoromethyl)benzamide (5.83 mmol) in THF was added (BOC)₂O (3.82 g, 17.5 mmol) and TEA (4.06 mL, 29.15 mmol). The solution was degassed with nitrogen and then Raney Ni was added. The system was degassed with hydrogen and then stirred at rt until TLC indicated complete reaction. The reaction mixture was filtered through celite and concentrated. The residue was purified by column chromatography to give tert-butyl({4-[3-(2-{[3-(trifluoromethyl)benzoyl]amino}ethyl)-phenoxylpyridin-2-yl]methyl)carbamate (I-50) as a white solid (2.0 g, 66%). ¹H NMR (300 MHz, CD₃OD) δ: 8.22 (d, 1H), 8.07 (s, 1H), 8.00 (d, 1H), 7.82 (d, 1H), 7.64 (t, 1H), 7.40 (t, 1H), 7.20 (d, 1H), 7.05 (s, 1H), 6.94-7.01 (m, 1H), 6.82-6.85 (m, 1H), 6.73-6.80 (m, 1H), 4.24 (br s, 2H), 3.65 (t, 2H), 2.96 (t, 2H), and 1.39 (s, 9H).

### Step 2: Preparation of N-[2-(3-{[2-(aminomethyl)pyridin-4-yl]oxy}phenyl)ethyl]-3-(trifluoromethyl)benzamide (I-45)

To a solution of tert-butyl({4-[3-(2-{[3-(trifluoromethyl)benzoyl]amino}ethyl)-phenoxy]pyridin-2-yl)methyl)carbamate (2.0) in DCM was added TFA (4.0 mL). The reaction mixture was allowed to stir at rt overnight and then concentrated. Purification by column chromatography gave N-[2-(3-{[2-(aminomethyl)pyridin-4-yl]oxy}phenyl)ethyl]-3-(trifluoromethyl)benzamide (I-45, 1.3 g). ¹H NMR (400 MHz, CD₃OD) δ: 8.78-8.82 (m, 1H), 8.38 (d, 1H), 8.05 (br s, 1H), 7.99 (d, 1H), 7.82 (d, 1H), 7.64 (t, 1H), 7.39 (t, 1H), 7.21 (d, 1H), 7.05-7.08 (m, 1H), 6.94-7.01 (m, 2H), 6.86 (dd, 1H), 4.16 (s, 2H), 3.62-3.69 (m, 2H), and 2.93-2.99 (m, 2H).

### Step 3: Preparation of N-[2-[3-({2-[(4,5-dihydro-1H-imidazol-2-ylamino)methyl]pyridin-4-yl}oxy)phenyl]ethyl}-3-(trifluoromethyl)benzamide (I-2)

To a solution of N-[2-(3-{[2-(aminomethyl)pyridin-4-yl]oxy}phenyl)ethyl]-3-(trifluoromethyl)benzamide (0.22 g, 0.51 mmol) in EtOH (9 mL) and AcOH (1 mL) was added tert-butyl 2-(methylsulfanyl)-4,5-dihydro-1H-imidazole-1-carboxylate (0.11 g, 0.51 mmol). The reaction mixture was heated at 65° C. overnight and then quenched by the addition of water. The solution was extracted with EtOAc and the organic solutions were combined, washed with brined, dried over Na₂SO₄, filtered, and concentrated. The residue was purified by column chromatography to give N-{2-[3-({2-[(4,5-dihydro-1H-imidazol-2-ylamino)methyl]pyridin-4-yl}oxy)phenyl]ethyl}-3-(trifluoromethyl)benzamide (I-2). ¹H NMR (300 MHz, CD₃OD) δ: 8.30 (d, 1H), 8.06 (br s, 1H), 8.01 (d, 1H), 7.82 (d, 1H), 7.64 (t, 1H), 7.40 (d, 1H), 7.22 (d, 1H), 7.05-7.08 (m, 1H), 6.96-7.01 (m, 1H), 6.91 (d, 1H), 6.80 (dd, 1H), 4.48 (s, 2H), 3.71 (s, 4H), 3.66 (t, 2H), and 2.97 (t, 2H).

The following compound was prepared from the appropriate starting materials in a method analogous to that of Example 5:
I-107 ¹H NMR (400 MHz, d₆-DMSO, HCOOH salt) δ: 8.80-8.88 (m, 1H), 8.38 (d, 1H), 8.30-8.35 (br s, 1H), 8.23-8.28 (m, 1H), 8.10-8.16 (m, 1H), 7.84 (d, 1H), 7.34-7.42 (m, 1H), 7.16 (d, 1H), 7.03-7.06 (m, 1H), 6.98-7.02 (m, 1H), 6.93-6.98 (m, 1H), 6.75-6.81 (m, 1H), 3.88 (s, 2H), 3.22-3.24 (m, 2H), 2.63-2.71 (m, 2H), and 1.79-1.89 (m, 2H).

### Example 6

### Synthesis of 3-cyano-N-[2-(3-{[2-(4,5-dihydro-1H-imidazol-2-yl)pyridin-4-yl]oxy}phenyl)ethyl]benzamide (I-63)

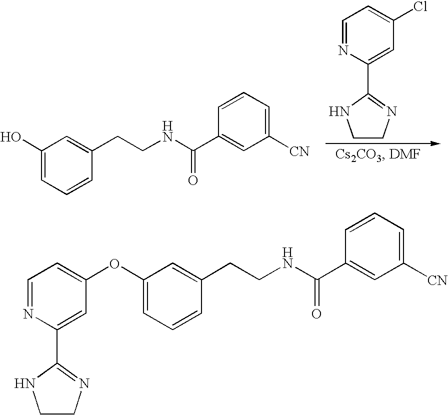

A mixture of 3-cyano-N-[2-(3-hydroxyphenyl)ethyl]benzamide (0.41 g, 1.5 mmol), 4-chloro-2-(4,5-dihydro-1H-imidazol-2-yl)pyridine (0.28 g, 1.5 mmol), and Cs₂CO₃ (1.4 g, 4.5 mmol) in DMF (15 mL) was heated at 100° C. overnight. The reaction mixture was allowed to cool to rt and then diluted with water and 1N NaOH. The solution was extracted with EtOAc and the organic solutions were combined, dried over MgSO₄, filtered, and concentrated. The residue was purified by column chromatography to give 3-cyano-N-[2-(3-{[2-(4,5-dihydro-1H-imidazol-2-yl)pyridin-4-yl]oxy}phenyl)ethyl]benzamide (I-63). ¹H NMR (300 MHz, d₆-DMSO) δ: 8.75-8.80 (m, 1H), 8.45 (d, 1H), 8.17-8.20 (m, 2H), 8.05-8.10 (m, 1H), 7.95-8.00 (m, 1H), 7.65 (t, 1H), 7.39-7.46 (m, 2H), 7.21 (d, 1H), 7.00-7.12 (m, 3H), 3.63 (s, 4H), 3.49-3.58 (m, 2H), and 2.86-2.93 (m, 2H).

The following compound was prepared from the appropriate starting materials in a method analogous to that of Example 6:

| | |
|---|---|
| I-21 | ¹H NMR (400 MHz, CD₃OD) δ: 8.53 (d, 1H), |
| | 7.49 (d, 1H), 7.41 (t, 1H), 7.22 (br d, |
| | 1H), 7.16 (dd, 1H), 7.09-7.12 (m, 1H), |
| | 6.99-7.04 (m, 2H), 6.77-6.83 (m, 2H), 4.01 (s, |
| | 4H), 3.78-3.82 (m, 4H), 3.63-3.68 (m, 2H), |
| | 3.12-3.18 (m, 4H), and 2.92-2.97 (m, 2H). |

### Example 7

### Expression and Purification of Raf Kinase Enzymes

Wild-Type B-Raf

Enzymatically active wild-type B-Raf was purchased from Upstate (cat# 14-530).

V599E B-Raf

Enzymatically active mutant B-Raf(V599E) was purchased from Upstate (cat# 14-557).

Wild Type C-Raf

Enzymatically active C-Raf was purchased from Upstate (cat# 14-352).

### Example 8

### Raf Kinase Enzyme Assays

B-Raf Flash Plate® Assay

Enzyme mix (15 μL), containing 50 mM HEPES pH 7.5, 0.025% Brij 35, 10 mM DTT, 4 nM B-Raf (V599E or Wild Type), was added to the wells of an assay plate and incubated for 20 minutes. Substrate mix (15 μL), containing 50 mM HEPES pH 7.5, 0.025% Brij 35, 10 mM MnCl₂, 2 μM Peptide 118 (Biotin-DRGFPRARYRARTTNYNSSR—SRFYSGFNSRPRGRVYRGRARATSWYSPY—NH₂, New England Peptide), 1 μM ATP, 0.2 mg/mL BSA, ³³P ATP 0.5 μCi/reaction was then added. Final reagent concentrations in the reaction mixture were 50 mM HEPES pH 7.5, 0.025% Brij 35, 5 mM DTT, 5 mM MnCl₂, 1 μM Peptide 118, 0.5 μM ATP, 0.1 mg/mL BSA, 2 nM B-Raf Wild Type, and ³³P ATP 0.5 μCi//reaction. The reaction mixture, with or without Raf kinase inhibitor, was incubated for 60 minutes, and then stopped by the addition of 50 μL of 100 mM EDTA. The stopped reaction mixture (65 μL) was transferred to a Flash Plate® (Perkin Elmer) and incubated for 2 hours. The wells were washed three times with 0.02% Tween-20. Plates were read on a TopCount analyzer.

Compounds I-1 to I-133 and I-192 to I-194 were tested in this assay. The following compounds exhibited IC₅₀ values less than or equal to 1 μM in this assay: I-2, I-8, I-12, I-13, I-15, I-16, I-20, I-26, I-28, I-29, I-31, I-32, I-38, I-44, I-50, I-53, I-54, I-55, I-57, I-58, I-59, I-64, I-68, I-73, I-82, I-85, I-87, I-92, I-98, I-104, I-110, I-116, I-121, I-122, I-126, I-129, I-130, I-132, and I-192.

The following compounds exhibited IC₅₀ values of greater than 1 μM and less than or equal to 10 μM in this assay: I-1, I-4, I-5, I-6, I-7, I-9, I-18, I-21, I-23, I-27, I-30, I-34, I-39, I-40, I-41, I-42, I-43, I-45, I-51, I-52, I-61, I-62, I-63, I-67, I-72, I-74, I-76, I-77, I-84, I-86, I-89, I-91, I-93, I-94, I-95, I-96, I-101, I-103, I-107, I-108, I-109, I-114, I-115, I-117, I-120, I-125, I-128, I-131, I-133, I-193, and I-194.

The following compounds produced 40-68% inhibition when tested at a concentration of 10 μM in this assay: I-3, I-10, I-11, I-14, I-17, I-19, I-22, I-24, I-25, I-33, I-35, I-36, I-37, I-46, I-47, I-49, I-56, I-60, I-65, I-66, I-69, I-70, I-71, I-75, I-78, I-79, I-81, I-83, I-88, I-99, I-100, I-102, I-105, I-106, I-111, I-112, I-118, I-119, I-124, I-127, I-134, I-135, I-136, I-137, I-138, I-139, I-140, I-141, I-142, I-143, I-144, I-145, I-146, I-147, I-148, I-149, I-150, I-151, I-152, I-153, I-154, I-155, I-156, I-157, I-158, I-159, I-160, I-161, I-162, I-163, I-164, I-165, I-166, I-167, I-168, I-169, I-170, I-171, I-172, I-173, I-174, I-175, I-176, I-177, I-178, I-179, I-180, I-181, I-182, I-183, I-184, I-185, I-186, I-187, I-188, I-189, I-190, and I-191.

C-Raf Flash Plate® Assay

Enzyme mix (15 μL), containing 50 mM HEPES pH 7.5, 0.025% Brij 35, 10 mM DTT, 20 nM C-Raf (Wild Type), was added to the wells of an assay plate and incubated for 20 minutes. Substrate mix (15 μL), containing 50 mM HEPES pH 7.5, 0.025% Brij 35, 10 mM MnCl₂, 4 μM Peptide 118, 1 μM ATP, 0.1 mg/mL BSA, ³³P ATP 0.5 μCi/reaction was then added. Final reagent concentrations in the reaction mixture were 50 mM HEPES pH 7.5, 0.025% Brij 35, 5 mM DTT, 5 mM MnCl₂, 2 μM Peptide 118, 1.0 μM ATP, 0.1 mg/mL BSA, 10 nM C-Raf Wild Type, and ³³P ATP 0.5 μCi//reaction. The reaction mixture was incubated for 40 minutes, and then stopped by the addition of 50 μL of 100 mM EDTA. The stopped reaction mixture (65 μL) was transferred to a Flash Plate® (Perkin Elmer) and incubated for 2 hours. The wells were washed three times with 0.02% Tween-20. Plates were read on a TopCount analyzer.

### Example 9

### Raf Kinase Cellular Assays

Phospho-ERK ELISA Assay

Inhibition of Raf kinase activity in whole cell systems can be assessed by determining the decrease in phosphorylation of Raf kinase substrates. Any known Raf kinase substrate can be used to measure inhibition of Raf kinase activity in a whole cell system.

In a specific example, A375 cells were seeded in a 96-well cell culture plate (12×10³ cells/100 μL/well) and incubated overnight at 37° C. Medium was removed, and cells were incubated with Raf kinase inhibitors for 3 hours at 37° C. Medium was removed, and cells were fixed with 4% paraformaldehyde for 15 minutes at room temperature.

Methanol was added for 15 min. Cells were removed and blocked with 10% sheep serum and 1% BSA in PBS overnight at 4° C. Cells were incubated with anti-p44/42MAPK antibody (1:100, Cell Signaling Technologies, #9101L) (20 μL/well) for one hour at room temperature. After washing with PBS three times, cells were stained with anti-rabbit horseradish peroxidase-linked antibody from donkey (1:100, Amersham Bioscience #NA934V) for 1 hour at room temperature. Cells were washed three times with 0.5% Tween-20 in PBS and twice with PBS. 3,3′,5,5′-Tetramethylbenzidine (TMB) liquid substrate system (Sigma, #T8665) (50 μL/well) was added, and cells were incubated for 30-45 minutes at room temperature. Optical density was read at 650 nm. Cells were then washed 3-5 times with PBS to remove color solution. Results were normalized for the protein content in each well using a BCA protein assay kit (Pierce).

### Example 10

### Anti-proliferation Assays

WST assay

A375 cells (4000) in 100 μL of 1% FBS-DMEM were seeded into wells of a 96-well cell culture plate and incubated overnight at 37° C. Test compounds were added to the wells and the plates were incubated for 48 hours at 37° C. Test compound solution was added (100 μL/well in 1% FBS DMEM), and the plates were incubated at 37° C. for 48 hours. WST-1 reagent (Roche #1644807, 10 μL) was added to each well and incubated for four hours at 37° C. as described by the manufacturer. The optical density for each well was read at 450 nm and 600 nm. A well containing medium only was used as a control.

### Example 11

### In vivo Assays

In Vivo Tumor Efficacy Model

Raf kinase inhibitors are tested for their ability to inhibit tumor growth in standard xenograft tumor models.

For example, HCT-116 cells (1×10⁶) in 100 μL of phosphate buffered saline are aseptically injected into the subcutaneous space in the right dorsal flank of female CD-1 nude mice (age 5-8 weeks, Charles River) using a 23-ga needle. Beginning at day 7 after inoculation, tumors are measured twice weekly using a vernier caliper. Tumor volumes are calculated using standard procedures (0.5×length×width²). When the tumors reach a volume of approximately 200 mm³, mice are injected i.v. in the tail vein with test compound (100 μL) at various doses and schedules. All control groups receive vehicle alone. Tumor size and body weight are measured twice a week, and the study is terminated when the control tumors reach approximately 2000 mm. Analogous procedures are followed for melanoma (A375 or A2058 cells), colon (HT-29 or HCT-116 cells), and lung (H460 cells) tumor models.

While the foregoing invention has been described in some detail for purposes of clarity and understanding, these particular embodiments are to be considered as illustrative and not restrictive. It will be appreciated by one skilled in the art from a reading of this disclosure that various changes in form and detail can be made without departing from the true scope of the invention, which is to be defined by the appended claims rather than by the specific embodiments.

The patent and scientific literature referred to herein establishes knowledge that is available to those with skill in the art. Unless otherwise defined, all technical and scientific terms used herein have the same meaning as commonly understood by one of ordinary skill in the art to which this invention belongs. The issued patents, applications, and references that are cited herein are hereby incorporated by reference to the same extent as if each was specifically and individually indicated to be incorporated by reference. In the case of inconsistencies, the present disclosure, including definitions, will control.

What is claimed is:

1. A compound of formula (I): 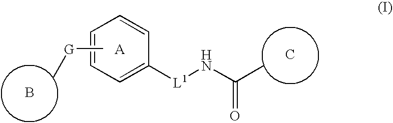
or a pharmaceutically acceptable salt thereof;
wherein:
G is —C(R^{d})(R^{e})—, —O—, —S—, or —N(R^{f})—, wherein G is attached to Ring A at the position meta or para to L¹;
L¹ is [C(R^{g})(R^{h})]ₘ—C(R^{j})(R^{k})—;
Ring A is substituted with 0-2 R^{aa};
Ring B is a 5- or 6-membered heteroaryl ring selected from the group consisting of 3-pyridyl, 4-pyridyl, 4-pyridazinyl, 4-pyrimidinyl, 5-pyrimidinyl, 2-oxazolyl, 4-oxazolyl, 5-oxazolyl, 2-thiazolyl, 4-thiazolyl, 5-thiazolyl, 2-imidazolyl, 4-imidazolyl, 5-imidazolyl, 3-isoxazolyl, 4-isoxazolyl, 5-isoxazolyl, 3-pyrazolyl, 4-pyrazolyl, 5-pyrazolyl, 2-pyrrolyl, and 3-pyrrolyl;
Ring B is substituted on its substitutable ring carbon atoms with 0-2 R^{bb} and 0-2 R^{8b};
each R^{bb} independently is halo, —NO₂, —CN, —C(R⁴)═C(R⁵)₂, —C≡C—R⁵, —R⁵, —SR⁶, —S(O)R⁶, —SO₂R⁶, —SO₂N(R⁴)₂, —N(R⁴)₂, —NR⁴C(O)R⁷, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OC(O)N(R⁴)₂—C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —C(═NR⁴)—N(R⁴)—OR⁵, —C(R⁶)═N—OR⁵, or an optionally substituted aliphatic, heteroaryl, or heterocyclyl;
each R^{8b} independently is selected from the group consisting of C₁₋₄ aliphatic, C₁₋₄ fluoroaliphatic, halo, —OH, —O(C₁₋₄ aliphatic), —NH₂, —NH(C₁₋₄ alkyl), and —N(C₁₋₄ alkyl)₂;
each substitutable ring nitrogen atom in Ring B is unsubstituted or is substituted with —C(O)R⁵, —C(O)N(R⁴)₂, —CO₂R⁶, —SO₂R⁶, —SO₂N(R⁴)₂, C₁₋₄ aliphatic, an optionally substituted C₆₋₁₀ aryl, or a C₆₋₁₀ ar(C₁₋₄)alkyl, the aryl portion of which is optionally substituted;
one ring nitrogen atom in Ring B optionally is oxidized;
Ring C is a 5- or 6-membered aryl or heteroaryl ring having 0-3 ring nitrogen atoms and optionally one additional ring heteroatom selected from oxygen and sulfur;
Ring C is substituted on its substitutable ring carbon atoms with 0-2 R^{cc} and 0-2 R^{8c};
each R^{cc} independently is halo, —NO₂, —CN, —C(R⁵)═C(R⁵)₂, —C≡C—R⁵, —OR⁵, —SR⁶, —S(O)R⁶, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —C(═NR⁴)—N(R⁴)—OR⁵, —C(R⁶)═N—OR⁵, or an optionally substituted aliphatic, aryl, heteroaryl, or heterocyclyl; or two adjacent R^{cc}, taken together with the intervening ring atoms, form a fused Ring E;
each R^{8c} independently is selected from the group consisting of C₁₋₄ aliphatic, C₁₋₄ fluoroaliphatic, —O(C₁₋₄ alkyl), —O(C₁₋₄ fluoroalkyl), and halo;
each substitutable ring nitrogen atom in Ring C is unsubstituted or is substituted with —C(O)R⁵, —C(O)N(R⁴)₂, —CO₂R⁶, —SO₂R⁶, —SO₂N(R⁴)₂, an optionally substituted C₆₋₁₀ aryl, or a C₁₋₄ aliphatic optionally substituted with —F, —OH, —O(C₁₋₄alkyl), —CN, —N(R⁴)₂, —C(O)(C₁₋₄ alkyl), —CO₂H, —CO₂(C₁₋₄ alkyl), —C(O)NH₂, —C(O)NH(C₁₋₄ alkyl), or an optionally substituted C₆₋₁₀ aryl ring;
one ring nitrogen atom in Ring C optionally is oxidized;
Ring E is a 5- or 6-membered aromatic or non-aromatic ring having 0-3 ring heteroatoms independently selected from the group consisting of O, N, and S;
each substitutable saturated ring carbon atom in Ring E is unsubstituted or is substituted with ═O, ═S, ═C(R⁵)₂, or —R^{ee};
each substitutable unsaturated ring carbon atom in Ring E is unsubstituted or is substituted with —R⁴;
each R^{ee} independently is halo, —NO₂, —CN, —C(R⁵)═C(R⁵)₂, —C≡C—R⁵, —OR⁵, —SR⁶, —S(O)R⁶, —SO₂R⁶, —SO₂N(R⁴)₂, —N(R⁴)₂, —NR⁴C(O)R⁷, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OC(O)N(R⁴)₂, —C(O)R⁵, —CO₂R⁷, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁴, —C(═NR¹)—N(R⁴)₂, —C(═NR⁴)—OR⁵, —C(═NR⁴)—N(R⁴)—OR⁵, —C(R⁶)═N—OR⁵, or an optionally substituted C₁₋₆ aliphatic;
each substitutable ring nitrogen atom in Ring E is unsubstituted or is substituted with —C(O)R⁵, —C(O)N(R⁴)₂, —CO₂R⁶, —SO₂R⁶, —SO₂N(R⁴)₂, C₁₋₄ aliphatic, an optionally substituted C₆₋₁₀ aryl, or a C₆₋₁₀ ar(C₁₋₄)alkyl, the aryl portion of which is optionally substituted;
one ring nitrogen or sulfur atom in Ring E optionally is oxidized;
R^{aa} is halo, —NO₂, —CN, —OR⁵, —SR⁶, —S(O)R⁶, —SO₂R⁶, —SO₂N(R⁴)₂, —N(R⁴)₂, —OC(O)R⁵, —CO₂R⁵, —C(O)N(R⁴)₂, —N(R⁴)SO₂R⁶, —N(R⁴)SO₂N(R⁴)₂, or a C₁₋₄ aliphatic or C₁₋₄ fluoroaliphatic optionally substituted with —OR⁵ or —N(R⁴)₂, provided that no more than one R^{aa} is —OH;
R^{d} is hydrogen, fluoro, C₁₋₄ aliphatic, C₁₋₄ fluoroaliphatic, —NH₂, —NH(C₁₋₄ alkyl), —N(C₁₋₄ alkyl)₂, OH, or —O(C₁₋₄ alkyl);
R^{e} is hydrogen, fluoro, C₁₋₄ aliphatic, or C₁₋₄ fluoroaliphatic; or R^{d} and R^{e}, taken together with the carbon atom to which they are attached, form a 3- to 6-membered cycloaliphatic or heterocyclyl ring;
R^{f} is —H, —C(O)R⁵, —C(O)N(R⁴)₂, —CO₂R⁶, —SO₂R⁶, —SO₂N(R⁴)₂, or an optionally substituted C₁₋₆ aliphatic;
R^{g} is hydrogen, fluoro, C₁₋₄ aliphatic, or C₁₋₄ fluoroaliphatic, and R^{h} is hydrogen, fluoro, C₁₋₄ aliphatic, C₁₋₄ fluoroaliphatic, —OH, —O(C₁₋₄ alkyl), —N(R⁴)₂, —N(R⁴)C(O)(C₁₋₄ aliphatic); or R^{g} and R^{h}, taken together with the carbon atom to which they are attached, form a 3- to 6-membered cycloaliphatic ring;
R^{j} is hydrogen, fluoro, C₁₋₄ aliphatic, or C₁₋₄ fluoroaliphatic, and R^{k} is hydrogen, fluoro, C₁₋₄ aliphatic, C₁₋₄ fluoroaliphatic, —C(O)(C₁₋₄ alkyl), —CO₂H, or —CO₂(C₁₋₄ alkyl); or R^{j} and R^{k}, taken together with the carbon atom to which they are attached, form a 3- to 6-membered cycloaliphatic ring; or
R^{g} and R^{j} are each hydrogen, fluoro, C₁₋₄ aliphatic, or C₁₋₄ fluoroaliphatic, and R^{k} and the vicinal R^{h}, taken together with the intervening carbon atoms, form a 3- to 6-membered cycloaliphatic ring;
each R⁴ independently is hydrogen or an optionally substituted aliphatic, aryl, heteroaryl, or heterocyclyl group; or two R⁴ on the same nitrogen atom, taken together with the nitrogen atom, form an optionally substituted 4- to 8-membered heterocyclyl ring having, in addition to the nitrogen atom, 0-2 ring heteroatoms independently selected from N, O, and S;
each R⁵ independently is hydrogen or an optionally substituted aliphatic, aryl, heteroaryl, or heterocyclyl group; and
each R⁶ independently is an optionally substituted aliphatic, aryl, or heteroaryl group; and
m is 1 or 2;
provided that Ring B is other than substituted or unsubstituted imidazolyl when Ring C is substituted or unsubstituted phenyl and G¹ is —CH₂— in the para position.

2. The compound of claim 1, characterized by one or more of the following features:
(a) each R^{aa} independently is —F, —Cl, —CN, —NO₂, C₁, alkyl, —CF₃, —O(C₁₋₄ alkyl), —OCF₃, —S(C₁₋₄ alkyl), —SO₂(C₁₋₄ alkyl), —NH₂, —NH(C_{—}4 alkyl), —N(C₁₋₄ alkyl)₂, —CO₂H, —C(O)NH₂, or —C(O)NH(C₁₋₄ alkyl);
(b) R^{h} and R^{k} are each independently hydrogen, fluoro, C₁₋₄ alkyl, or C₁₋₄ fluoroalkyl;
(c) L¹ is —CH₂—CH₂— or —CH₂—CH₂—CH₂—; and
(d) G is —O— or —NH—.

3. The compound of claim 2, wherein each R^{bb} independently is selected from the group consisting of C₁₋₆ aliphatic, C₁₋₆ fluoroaliphatic, halo, —R^{2b}, -T¹-R^{1b}, -T¹-R^{2b}, —V¹-T¹-R^{1b}, —V¹-T¹-R^{2b}, optionally substituted heterocyclyl, and optionally substituted heteroaryl;
T¹ is a C₁₋₆ alkylene chain optionally substituted with R^{3a} or R^{3b}, wherein the alkylene chain optionally is interrupted by —C(R⁵)═C(R⁷)—, —C≡C—, —O—, —S—, —S(O)—, —S(O)₂—, —SO₂ N(R⁴)—, —N(R⁴)—, —N(R⁴)C(O)—, —NR⁴C(O)N(R⁴)—, —N(R⁴)C(═NR⁴)—N(R⁴)—, —N(R⁴)—C(═NR⁴)—, —N(R⁴)CO₂—, —N(R⁴)SO₂—, —N(R⁴)SO₂N(R⁴)—, —OC(O)—, —OC(O)N(R⁴)—, —C(O)—, —CO₂—, —C(O)N(R⁴)—, —C(═NR⁴)—N(R⁴)—, —C(NR⁴)═N(R⁴)—, —C(═NR⁴)—O—, or —C(R⁶)═N—O—, and wherein T or a portion thereof optionally forms part of a 3-7 membered ring;
V¹ is —C(R⁴)═C(R⁵)—, —C≡C—, —O—, —S—, —S(O)—, —S(O)₂—, —SO₂N(R⁴)—, —N(R⁴)—, —N(R⁴)C(O)—, —NR⁴C(O)N(R⁴)—, —N(R⁴)C(═NR⁴)—N(R⁴)—, —N(R⁴)C(═NR⁴)—, —N(R⁴)CO₂—, —N(R⁴)SO₂—, —N(R⁴)SO₂N(R⁴)—, —OC(O)—, —OC(O)N(R⁴)—, —C(O)—, —CO₂—, —C(O)N(R⁴)—, —C(O)N(R⁴)—O—, —C(O)N(R⁴)C(═NR⁴)—N(R⁴)—, —N(R⁴)C(═NR⁴)—N(R⁴)—C(O)—, —C(═NR¹)—N(R⁴)—, —C(NR⁴)═N(R⁴)—, —C(═NR⁴)—O—, or —C(R⁶)═N—O—;
each R^{1b} independently is an optionally substituted aryl, heteroaryl, heterocyclyl, or cycloaliphatic ring;
each R^{2b} independently is —NO₂, —CN, —C(R⁵)═C(R⁵)₂, —C≡C—R⁵, —OR⁵, —SR⁶, —S(O)R⁶, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂—N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —C(═NR⁴)—N(R⁴)—OR⁵, or —C(R⁶)═N—OR⁵;
each R^{3a} independently is selected from the group consisting of —F, —OH, —O(C₁₋₄ alkyl), —CN, —N(R⁴)₂, —C(O)(C₁₋₄ alkyl), —CO₂H, —CO₂(C₁₋₄ alkyl), —C(O)NH₂, and —C(O)NH(C₁₋₄ alkyl);
each R^{3b} independently is a C₁₋₃ aliphatic optionally substituted with R^{3a} or R⁷, or two substituents R^{3b} on the same carbon atom, taken together with the carbon atom to which they are attached, form a 3- to 6-membered cycloaliphatic ring; and
each R⁷ independently is an optionally substituted aryl or heteroaryl ring.

4. The compound of claim 3, wherein Ring B is an optionally substituted pyrimidinyl, pyridyl, or N-oxidopyridyl.

5. The compound of claim 4, wherein the substitutable ring carbon atoms in Ring B are substituted with 0-1 R^{bb} and 0-1 R^{8b};
R^{bb} is selected from the group consisting of C₁₋₄ aliphatic, C₁₋₄ fluoroaliphatic, halo, —R^{2b}, -T¹-R^{1b}-T¹-R^{2b}, —V¹-T¹-R^{1b}, —V¹-T¹-R^{2b}, optionally substituted heteroaryl, and optionally substituted heterocyclyl;
T¹ is a C₁₋₄ alkylene chain optionally substituted with one or two substituents independently selected from the group consisting of C₁₋₃ aliphatic, C₁₋₃ fluoroaliphatic, —F, —OH, —O(C₁₋₄ alkyl), —CO₂H, —CO₂(C₁₋₄alkyl), —C(O)NH₂, and —C(O)NH(C₁₋₄ alkyl), wherein the alkylene chain optionally is interrupted with —N(R⁴)—, —C(═NR⁴)—N(R⁴)—, —C(NR⁴)═N(R⁴)—, —N(R⁴)—C(═NR⁴)—, —N(R⁴)—C(O)—, or —C(O)N(R⁴)—;
V¹ is —C(R⁵)═C(R⁵)—, —C≡C—, —O—, —N(R⁴)—, —N(R⁴)C(O)—, —C(O)N(R⁴)—, —C(═NR⁴)—N(R⁴)—, —C(NR⁴)═N(R⁴)—, or —N(R⁴)—C(═NR⁴)—;
each R^{1b} independently is an optionally substituted aryl, heteroaryl, heterocyclyl, or cycloaliphatic ring;
each R^{2b} independently is —NO₂, —CN, —C(R⁵)═C(R⁵)₂1-C≡C—R⁵, —OR⁵, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶—NR⁴C₂R⁶, —N(R⁴)SO₂R⁶, —N(R⁴)SO₂N(R⁴)₂, —O—C(O)R⁵, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —C(═NR⁴)—N(R⁴)—OR⁵, or —C(R⁶)═N—OR⁵; and
R^{8b} is selected from the group consisting of Cl₄ aliphatic, C₁₋₄ fluoroaliphatic, halo, —OH, —O(C₁₋₄ aliphatic), —NH₂, —NH(C₁₋₄ aliphatic), and —N(C₁₋₄ aliphatic)₂.

6. The compound of claim 2, having formula (II): 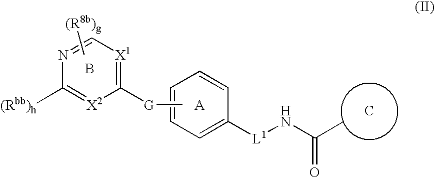
or a pharmaceutically acceptable salt thereof;
wherein:
G is —O— or —NH—;
X¹ and X² are each independently CH or N, provided that X¹ and X are not both N;
one ring nitrogen atom in Ring B optionally is oxidized;
R^{bb} is selected from the group consisting of halo, —N(R⁴)₂, —CO₂R⁵, —C(O)—N(R⁴)₂, —C(O)—N(R⁴)—OR⁵, —N(R⁴)C(O)R⁵, —N(R⁴)C(O)—OR⁵, —N(R⁴)C(O)—N(R⁴)₂, —N(R⁴)SO₂R⁶, —C(═NR⁴)N(R⁴)₂, and —C(═NR⁴)N(R⁴)—OR⁵;
R^{8b} is selected from the group consisting of C₁₋₄ aliphatic, C₁₋₄ fluoroaliphatic, halo, —OH, —O(C₁₋₄ aliphatic), —NH₂, —NH(C₁₋₄ aliphatic), and —N(C₁₋₄ aliphatic)₂;
g is 0 or 1; and
h is 0 or 1.

7. The compound of claim 6, wherein:
R^{bb} is selected from the group consisting of halo, —N(R^{4x})(R^{4z}), —CO₂R^{5x}, —C(O)—N(R^{4x})(R^{4z}), —C(O)—N(R^{4x})—OR^{5x}, —N(R^{4x})C(O)R^{5x}, —N(R^{4x})C(O)—OR^{5x}, —N(R^{4x})C(O)—N(R^{4x})(R^{4z}), —N(R^{4x})SO₂R^{6x}, —C(═NR^{4x})N(R^{4x})(R^{4z}), and —C(═NR^{4x})N(R^{4x})—OR^{5x};
R^{4x} is hydrogen, C₁₋₄ alkyl, C₁₋₄ fluoroalkyl, or C₆₋₁₀ ar(C₁₋₄)alkyl, the aryl portion of which may be optionally substituted;
R^{4z} is hydrogen, C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring; or
R^{4x} and R^{4z}, taken together with the nitrogen atom to which they are attached, form an optionally substituted 4- to 8-membered heterocyclyl ring having, in addition to the nitrogen atom, 0-2 ring heteroatoms independently selected from N, O, and S;
each R^{5x} independently is hydrogen, C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring; and
each R^{6x} independently is C₁₋₄alkyl, C₁₋₄ fluoroalkyl, C₁₋₁₀ ar(C₁₋₄)alkyl, the aryl portion of which may be optionally substituted, or an optionally substituted 5- or 6-membered aryl, heteroaryl, or heterocyclyl ring.

8. The compound of claim 7, wherein R^{bb} is selected from the group consisting of —N(R^{4x})(R^{4z}), —CONH(R^{4z}), —NHC(O)(R^{5x}), and —C(═NH)N(R^{4x})(R^{4z}).

9. The compound of claim 2, having the formula (II): 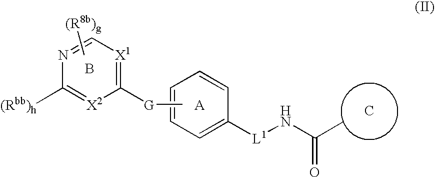
or a pharmaceutically acceptable salt thereof;
wherein:
G is —O— or —NH—;
X¹ and X² are each independently CH or N, provided that X¹ and X² are not both N;
one ring nitrogen atom in Ring B optionally is oxidized;
R^{bb} is —V¹-T¹-R^{1b} or —V¹-T¹-R^{2b};
V¹ is —N(R⁴)—, —N(R⁴)—C(O)—, —N(R⁴)SO₂R⁶—N(R⁴)C(O)—OR⁵, —C(O)N(R⁴)—, —C(═NR⁴)N(R⁴)—, or —N(R⁴)—C(═NR⁴)—;
T¹ is a C₁₋₄ alkylene chain optionally substituted with —F, C₁₋₃ alkyl, or C₁₋₃ fluoroalkyl;
R^{1b} is an optionally substituted C₃₋₆ cycloaliphatic or an optionally substituted phenyl, pyrrolyl, imidazolyl, oxazolyl, thiazolyl, isoxazolyl, isothiazolyl, pyrazolyl, triazolyl, tetrazolyl, oxadiazolyl, thiadiazolyl, pyrrolinyl, imidazolinyl, pyrazolinyl, pyrrolidinyl, imidazolidinyl, pyrazolidinyl, piperidinyl, morpholinyl, piperazinyl, pyridyl, pyridazinyl, pyrimidinyl, pyrazinyl, or tetrahydropyrimidinyl ring;
R^{2b} is —N(R⁴)₂, —NR⁴C(O)R⁵, —N(R⁴)C(O)—OR⁵, —N(R⁴)C(O)—N(R⁴)₂, —C(O)N(R⁴)₂, —CO₂R⁵, or —OR⁵;
R^{8b} is selected from the group consisting of C₁₋₄ aliphatic, C₁₋₄ fluoroaliphatic, halo, —OH, —O(C₁₋₄ aliphatic), —NH₂, —NH(C₁₋₄ aliphatic), and —N(C₁₋₄ aliphatic)₂;
g is 0 or 1; and
h is 0 or 1.

10. The compound of claim 9, wherein:
V¹ is —N(R^{4x})—, —N(R^{4x})—C(O)—, —C(O)N(R^{4x})—, —C(═NR^{4x})N(R^{4x})—, or —N(R^{4x})—C(═NR^{4x})—;
R^{1b} is an optionally substituted C₃₋₆ cycloaliphatic or an optionally substituted pyrrolidinyl, piperidinyl, morpholinyl, or piperazinyl; and
R^{2b} is —N(R^{4x})(R^{4z}), —NR^{4x}C(O)R^{5x}, —C(O)N(R^{4x})(R^{4z}), —CO₂R^{5x}, or —OR^{5x};
R^{4x} is hydrogen, C₁₋₄ alkyl, C₁₋₄ fluoroalkyl, or C₆₋₁₀ ar(C₁₋₄)alkyl, the aryl portion of which may be optionally substituted;
R^{4z} is hydrogen, C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring; or
R^{4x} and R^{4z}, taken together with the nitrogen atom to which they are attached, form an optionally substituted 4 to 8-membered heterocyclyl ring having, in addition to the nitrogen atom, 0-2 ring heteroatoms independently selected from N, O, and S; and
each R^{5x} independently is hydrogen, C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring.

11. The compound of claim 10, wherein X and X² are each CH and V¹ is —C(O)—NH— or —NH—C(O)—.

12. The compound of claim 11, wherein R^{bb} is selected from the group consisting of: 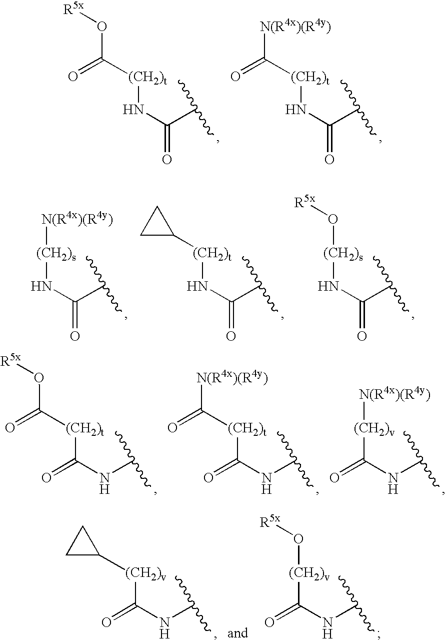
is 2 or 3;
t is 1, 2, or 3; and
v is 0, 1, 2, or 3.

13. The compound of claim 2, having the formula (II): 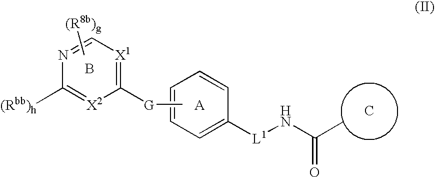
or a pharmaceutically acceptable salt thereof;
wherein:
G is —O— or —NH—;
X¹ and X² are each independently CH or N, provided that X¹ and X² are not both N;
one ring nitrogen atom in Ring B optionally is oxidized;
R^{bb} is -T¹-R^{1b} or -T¹-R^{2b}
T¹ is a C₁₋₆ alkylene chain optionally substituted with —F, C₁₋₃ alkyl, or C₁₋₃ fluoroalkyl, wherein the alkylene chain optionally is interrupted by —N(R⁴)—, —C(O)—N(R⁴)—, —C(═NR⁴)—N(R⁴)—, —N(R⁴)—C(O)—, or —N(R⁴)—C(═NR⁴)—;
R^{1b} is an optionally substituted C₃₋₆ cycloaliphatic or an optionally substituted phenyl, pyrrolyl, imidazolyl, oxazolyl, thiazolyl, isoxazolyl, isothiazolyl, pyrazolyl, triazolyl, tetrazolyl, oxadiazolyl, thiadiazolyl, pyrrolinyl, imidazolinyl, pyrazolinyl, pyrrolidinyl, imidazolidinyl, pyrazolidinyl, piperidinyl, morpholinyl, piperazinyl, pyridyl, pyridazinyl, pyrimidinyl, pyrazinyl, or tetrahydropyrimidinyl ring;
R^{2b} is —OR⁵, —N(R⁴)₂, —NR⁴C(O)R⁵, —NR⁴C(O)N(R⁴)₂, —C(O)N(R⁴)—OR⁵, —C(O)N(R⁴)₂, —N(R⁴)—CO₂R⁵, —N(R⁴)—C(═NR⁴)—R⁵ or —C(═NR⁴)—N(R⁴)₂;
R^{8b} is selected from the group consisting of C₁₋₄ aliphatic, C₁₋₄ fluoroaliphatic, halo, —OH, —O(C₁₋₄ aliphatic), —NH₂, —NH(C₁₋₄ aliphatic), and —N(C₁₋₄ aliphatic)₂;
g is 0 or 1; and
h is 0 or 1.

14. The compound of claim 13, wherein:
R^{bb} is selected from the group consisting of —(CH₂)_{q}R^{1x}, —(CH₂)_{q}—R^{2x}, —(CH₂)_{q}—R^{2y} —(CH₂)_{q}—N(R^{4x})—(CH₂)_{q}—R^{1x}, —(CH₂)_{q}—N(R^{4x})—(CH₂)_{q}—R^{2x}, —(CH₂)_{q}—N(R^{4x})—(CH₂)ₛ—R^{2y} —(CH₂)_{q}—N(R^{4x})C(═NR^{4x})—(CH₂)_{q}—R^{1x}, —(CH₂)_{q}—N(R^{4x})C(═NR^{4x})—(CH₂)_{q}—R^{2x}—(CH₂)_{q}—N(R^{4x})C(═NR^{4x})—(CH₂)_{q}—R^{2y};
R^{1x} is an optionally substituted phenyl, piperidinyl, piperazinyl, morpholinyl, or pyrrolidinyl ring;
R^{2x} is —C(O)N(R^{4x})(R^{4z});
R^{2y} is —N(R^{4x})(R^{4z}), —NR^{4x}C(O)R^{5x}, —N(R^{4x})CO₂R^{5x}, —N(R^{4x})—C(═NR^{4x})—R^{5x} or —OR^{5x};
R^{4x} is hydrogen, C₁₋₄ alkyl, C₁₋₄ fluoroalkyl, or C₆₋₁₀ ar(C₁₋₄)alkyl, the aryl portion of which may be optionally substituted;
R^{4z} is hydrogen, C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring; or
R^{4x} and R^{4z}, taken together with the nitrogen atom to which they are attached, form an optionally substituted morpholinyl, piperidinyl, piperazinyl, or pyrrolidinyl ring;
R^{5x} is hydrogen, C₁₋₄ alkyl, C₁₋₄ fluoroalkyl, or C₆₋₁₀ ar(C₁₋₄)alkyl, the aryl portion of which may be optionally substituted.
R^{8b} is selected from the group consisting of C₁₋₄ aliphatic, C₁₋₄ fluoroaliphatic, halo, —OH, —O(C₁₋₄ aliphatic), —NH₂, —NH(C₁₋₄ aliphatic), and —N(C₁₋₄ aliphatic)₂;
q, at each occurrence independently, is 1, 2, or 3; and
s is 2 or 3.

15. The compound of claim 2, having the formula (III): 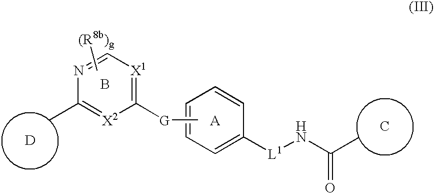
or a pharmaceutically acceptable salt thereof;
wherein:
G is —O— or —NH—;
X¹ and X² are each independently CH or N, provided that X¹ and X² are not both N;
one ring nitrogen atom in Ring B optionally is oxidized;
Ring D is an aryl, heteroaryl, heterocyclyl, or cycloaliphatic ring;
each substitutable saturated ring carbon atom in Ring D is unsubstituted or is substituted with ═O, ═S, ═C(R⁵)₂, ═N—OR⁵, ═N—R⁵, or —R^{dd};
each substitutable unsaturated ring carbon atom in Ring D is unsubstituted or is substituted with —R^{dd};
each substitutable ring nitrogen atom in Ring D is unsubstituted or is substituted with —C(O)R⁴, —C(O)N(R⁴)₂, —CO₂R⁶, —SO₂R⁶, —SO₂(NR⁴)₂, an optionally substituted C₁₋₁₀ aryl, or a C₁₋₄ aliphatic optionally substituted with R³ or R⁷;
one ring nitrogen atom in Ring D optionally is oxidized;
each R^{dd} independently is halo, —NO₂, —CN, —C(R⁵)═C(R⁵)₂, —C≡C—R⁵, —R⁵, —SR⁶, —S(O)R⁶, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —C(═NR⁴)—N(R⁴)—OR⁷, —C(R⁶)═N—OR⁵, or an optionally substituted aliphatic, aryl, heteroaryl, or heterocyclyl;
R^{8b} is selected from the group consisting of C₁₋₄ aliphatic, C₁₋₄ fluoroaliphatic, halo, —OH, —O(C₁₋₄ aliphatic), —NH₂, —NH(Cl₄ aliphatic), and —N(Cl₁₄ aliphatic)₂; and
g is 0 or 1.

16. The compound of claim 15, wherein Ring D is an optionally substituted heteroaryl or heterocyclyl selected from the group consisting of azetidinyl, pyrrolyl, imidazolyl, oxazolyl, thiazolyl, isoxazolyl, isothiazolyl, pyrazolyl, triazolyl, tetrazolyl, oxadiazolyl, thiadiazolyl, pyrrolinyl, imidazolinyl, pyrazolinyl, pyrrolidinyl, imidazolidinyl, pyrazolidinyl, piperidinyl, morpholinyl, piperazinyl, pyridyl, pyridazinyl, pyrimidinyl, pyrazinyl, and tetrahydropyrimidinyl.

17. The compound of claim 16, wherein:
Ring D is substituted with 0-1 R^{dd} and 0-1 R^{8d};
R^{dd} is selected from the group consisting of C₁₋₄ aliphatic, C₁₋₄ fluoroaliphatic, halo, —R^{1d}, —R^{2d}, -T³-R^{1d}, -T³-R^{2d}, —V³—R^{1d}, and —V³-T³-R^{2d};
T³ is a C₁₋₄ alkylene chain optionally substituted with one or two substituents independently selected from the group consisting of C₁₋₃ aliphatic, C₁₋₃ fluoroaliphatic, —F, —OH, —O(C₁₋₄ alkyl), —CO₂H, —CO₂(C₁₋₄ alkyl), —C(O)NH₂, and —C(O)NH(C₁₋₄ alkyl);
V³ is —O—, —N(R⁴)—, —N(R⁴)C(O)—, —C(O)N(R⁴)—, —C(═NR⁴)—N(R⁴)—, —C(NR⁴)═N(R⁴)—, or —N(R⁴)C(═NR⁴)—;
each R^{1d} independently is an optionally substituted aryl, heteroaryl, heterocyclyl, or cycloaliphatic ring;
each R^{2d} independently is —NO₂, —CN, —C(R⁵)═C(R⁵)₂, —C≡C—R⁵, —OR⁵, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁵, —N(R⁴)SO₂N(R⁴)₂, —O—C(O)R⁵, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —C(═NR⁴)—N(R⁴)—OR⁵, or —C(R⁶)═N—OR⁵; and
R^{8d} is C₁₋₄ aliphatic, Cl_fluoroaliphatic, halo, —OH, —O(C₁₋₄ aliphatic), —NH₂, —NH(C₁₋₄ aliphatic), or —N(C₁₋₄ aliphatic)₂.

18. The compound of claim 17, wherein each R^{2d} independently is selected from the group consisting of —OR⁵, —N(R⁴)₂, —NR⁴C(O)R⁵, —NR⁴C(O)N(R⁴)₂, —O—C(O)R⁵—CO₂R⁵, —C(O)R⁵, —C(O)N(R⁴)₂, —C(O)N(R⁴)—OR⁵, and —C(═NR⁴)—N(R⁴)₂.

19. The compound of claim 15, wherein Ring D is selected from the group consisting of: 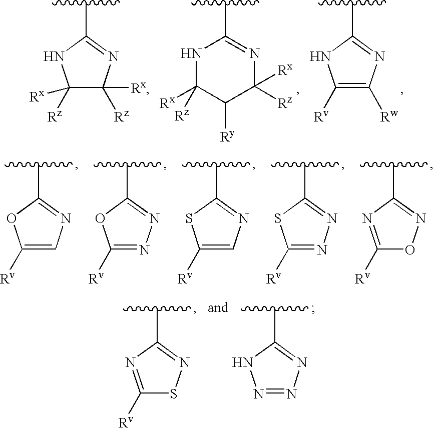
R^{v} is hydrogen, halo, C₁₋₄ aliphatic, C₁₋₄ fluoroaliphatic, —OR⁵, —N(R⁴)₂, —CO₂R⁵, —C(O)N(R⁴)₂, -T³-OR⁵, -T³-N(R⁴)₂, -T³-CO₂R⁵, -T³-C(O)N(R⁴)₂, or an optionally substituted 5- or 6-membered aryl or heteroaryl;
R^{w} is hydrogen, halo, C₁₋₄ aliphatic, C₁₋₄ fluoroaliphatic, —OR⁵, —N(R⁴)₂, —CO₂R⁵, or —C(O)N(R⁴)₂;
each R^{x} independently is hydrogen, fluoro, C₁₋₄ aliphatic, C₁₋₄ fluoroaliphatic, —CO₂R⁵, —C(O)N(R⁴)₂, -T³-N(R⁴)₂, -T³-OR⁵, -T³-CO₂R⁵, or -T³-C(O)N(R⁴)₂;
R^{y} is hydrogen, halo, C₁₋₄ aliphatic, C₁₋₄ fluoroaliphatic, —OR⁵, —N(R⁴)₂, —CO₂R⁵, —C(O)N(R⁴)₂, -T³-OR⁵, -T³-N(R⁴)₂, -T³-CO₂R⁵, or -T³-C(O)N(R⁴)₂;
each R^{z} independently is hydrogen, fluoro, C¹⁻⁴ aliphatic, or C¹⁻⁴ fluoroaliphatic; and
T³ is a C₁₋₄ alkylene chain optionally substituted with one or two substituents independently selected from the group consisting of C₁₋₃ aliphatic, C₁₋₃ fluoroaliphatic, —F, —OH, —O(C₁₋₄ alkyl), —CO₂H, —CO₂(C₁₋₄ alkyl), —C(O)NH₂, and —C(O)NH(C₁₋₄ alkyl);

20. The compound of claim 15, wherein Ring D is selected from the group consisting of: 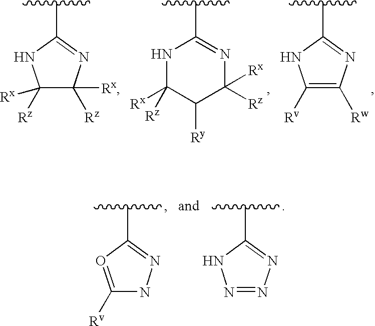
R^{v} is hydrogen, an optionally substituted phenyl, pyridyl, or pyrimidinyl group, halo, C₁₋₄ aliphatic, C₁₋₄ fluoroaliphatic, —(CH₂)ₚ—OR^{5x}, —(CH₂)ₚ—N(R^{4x})(R^{4z}), —(CH₂)ₚ—CO₂R^{5x}, —(CH₂)ₚ—C(O)N(R^{4x})(R^{4z}), —(CH₂)_{q}—N(R^{4x})—(CH₂)_{q}—R^{4x}, —(CH₂)_{q}—N(R^{4x})—(CH₂)_{q}—R^{2x}, —(CH₂)_{q}—N(R^{4x})—(CH₂), —R^{2x}—(CH₂)_{q}—N(R^{4x})C(═NR^{4x})—(CH₂)_{q}—R^{2y}, —(CH₂)_{q}—N(R^{4x})C(═NR^{4x})—(CH₂)_{q}—R^{2x} or —(CH₂)_{q}—N(R^{4x})C(═NR^{4x})(CH₂)_{q}—R^{2y};
R^{w} is hydrogen, halo, C₁₋₄ aliphatic, C₁₋₄ fluoroaliphatic, —OR⁵, —N(R⁴)₂, —CO₂R⁵, or —C(O)N(R⁴)₂;
each R^{x} independently is hydrogen, fluoro, C₁₋₄ aliphatic, C₁₋₄ fluoroaliphatic, —(CH₂)ₚ—CO₂R^{5x}, —(CH₂)ₚ—C(O)N(R^{4x})(R^{4z}), —(CH₂)ᵣ—N(R^{4x})(R^{4z}), or —(CH₂), —OR;
R^{y} is hydrogen, fluoro, C₁₋₄ aliphatic, C₁₋₄ fluoroaliphatic, —(CH₂)ₚ—N(R^{4x})(R^{4z}), —(CH₂)ₚ—OR^{5x}, —(CH₂)ₚ—CO₂R^{5x}, —(CH₂)ₚ—C(O)N(R^{4x})(R^{4z});
each R^{z} independently is hydrogen, fluoro, C₁₋₄ aliphatic, or C₁₋₄ fluoroaliphatic;
each R^{1x} independently is an optionally substituted phenyl, piperidinyl, piperazinyl, morpholinyl, or pyrrolidinyl ring;
each R^{2x} independently is —C(O)N(R^{4x})(R^{4z});
each R^{2y} independently is —N(R^{4x})(R^{4z}), —NR^{4x}C(O)R^{5x}, —N(R^{4x})—CO₂R^{5x}, —N(R^{4x})—C(═NR^{4x})—R^{5x} or —OR^{5x};
each R^{4x} independently is hydrogen, C₁₋₄ alkyl, C₁₋₄ fluoroalkyl, or C₆₋₁₀ ar(C₁₋₄)alkyl, the aryl portion of which may be optionally substituted;
each R^{4z} independently is hydrogen, C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring; or
R^{4x} and R^{4z}, taken together with the nitrogen atom to which they are attached, form an optionally substituted 4- to 8-membered heterocyclyl ring having, in addition to the nitrogen atom, 0-2 ring heteroatoms independently selected from N, O, and S;
each R^{5x} independently is hydrogen, C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring;
p is 0, 1, or 2;
q, at each occurrence independently, is 1, 2, or 3;
r is 1 or 2; and
s is 2 or 3.

21. The compound of claim 20, wherein:
X¹ and X² are each CH; and
Ring D is selected from the group consisting of:

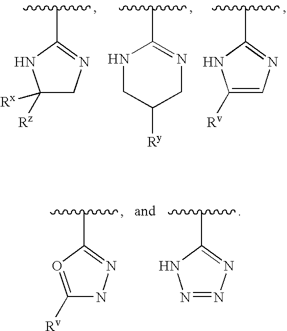

22. The compound of claim 2, wherein Ring C is an optionally substituted phenyl, furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, or triazinyl, wherein one ring nitrogen atom in Ring C is optionally oxidized.

23. The compound of claim 22, wherein each R^{cc} independently is selected from the group consisting of C₁₋₆ aliphatic, C₁₋₆ fluoroaliphatic, halo, —R^{1c}, —R^{2c}, -T²-R^{2c}, and -T²R^{1c}; or two adjacent R^{cc}, taken together with the intervening ring atoms, form a fused Ring E;
T² is a C₁₋₆ alkylene chain optionally substituted with R^{3a} or R^{3b}, wherein the alkylene chain optionally is interrupted by —C(R⁵)═C(R⁵)—, —C≡C—, —O—, —S—, —S(O)—, —S(O)₂—, —SO₂ N(R⁴)—, —N(R⁴)—, —N(R⁴)C(O)—, —NR⁴C(O)N(R⁴)—, —N(R⁴)CO₂—, —N(R⁴)SO₂—, —C(O)N(R⁴)—, —C(O)—, —CO₂—, —OC(O)—, or —OC(O)N(R⁴)—, and wherein T² or a portion thereof optionally forms part of a 3-7 membered ring;
each R^{1c} independently is an optionally substituted aryl, heteroaryl, heterocyclyl, or cycloaliphatic ring;
each R^{2c} independently is —NO₂, —CN, —C(R⁵)═C(R⁵)₂, —C≡C—R⁵, —OR⁵, —SR⁶, —S(O)R⁶, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)S₂N(R⁴)₂, —O—C(O)R⁵—OC(O)N(R⁴)₂, —C(O)R⁵, —CO₂R⁵, —C(O)N(R⁴)₂, —C(O)N(R⁴)—OR¹, —C(O)N(R⁴)C(═NR¹)—N(R⁴)₂, —N(R⁴)C(═NR⁴)—N(R⁴)—C(O)R⁵, —C(═NR⁴)—N(R⁴)₂, —C(═NR⁴)—OR⁵, —C(═NR⁴)—N(R⁴)—OR⁵, or —C(R⁶)═N—OR⁵;
each R^{3a} independently is selected from the group consisting of —F, —OH, —O(C₁₋₄ alkyl), —CN, —N(R⁴)₂, —C(O)(C₁₋₄ alkyl), —CO₂H, —CO₂(C₁₋₄ alkyl), —C(O)NH₂, and —C(O)NH(C₁₋₄ alkyl);
each R^{3b} independently is a C₁₋₃ aliphatic optionally substituted with R^{3a} or R⁷, or two substituents R^{3b} on the same carbon atom, taken together with the carbon atom to which they are attached, form a 3- to 6-membered cycloaliphatic ring; and
each R⁷ independently is an optionally substituted aryl or heteroaryl ring;

24. The compound of claim 2, wherein:
Ring C is a 5- or 6-membered heteroaryl substituted with 0-2 R^{cc}; and
each R^{cc} independently is selected from the group consisting of -halo, C₁₋₄ alkyl, C₁₋₄ fluoroalkyl, —O(C₁₋₄ alkyl), and —O(C₁₋₄ fluoroalkyl), or two adjacent R^{cc}, taken together with the intervening ring atoms, form an optionally substituted fused Ring E.

25. The compound of claim 2, wherein:
Ring C is phenyl substituted with 0-2 R^{cc} and 0-1 R^{8c};
each R^{cc} independently is selected from the group consisting of Cl₄ aliphatic, C₁₋₄ fluoroaliphatic, halo, —R^{2c} and -T¹-R^{2c}; or two adjacent R^{cc}, taken together with the intervening ring atoms, form a fused Ring E;
T² is a C₁₋₄ alkylene chain optionally substituted with one or two groups independently selected from —F, C₁₋₄ aliphatic, and C₁₋₄ fluoroaliphatic; and
each R^{2c} independently is —CN, —C(R⁵)═C(R⁵)₂, —C≡C—R⁵, —OR⁵, —SR⁶, —N(R⁴)₂, —NR⁴C(O)R⁵, —NR⁴C(O)N(R⁴)₂, —NR⁴CO₂R⁶, —CO₂R⁵, or —C(O)N(R⁴)₂; and
each R⁸ independently is selected from the group consisting of C, 4 aliphatic, C₁₋₄ fluoroaliphatic, —O(C₁₋₄ alkyl), —O(C₁₋₄ fluoroalkyl), and halo.

26. The compound of claim 25, wherein:
each R^{cc} independently is halo, —CN, —C(R^{5x})═C(R^{5x})(R^{5y}), C≡C—R^{5y}, —OR⁵y′-SR^{6x}, —CO₂R^{5x} —C(O)N(R^{4x})(R^{4y}), or a C₁₋₄ aliphatic or C₁₋₄ fluoroaliphatic optionally substituted with one or two substituents independently selected from the group consisting of —OR⁵), —N(R^{4x})(R^{4y}), —SR^{6x}, —CO₂R^{5x}, or —C(O)N(R^{4x})(R^{4y}); or two adjacent R^{cc}, taken together with the intervening ring atoms, form a fused Ring E;
R^{4x} is hydrogen, C₁₋₄ alkyl, C₁₋₄ fluoroalkyl, or C₆₋₁₀ ar(C₁₋₄)alkyl, the aryl portion of which may be optionally substituted, or two R^{4x} on the same nitrogen atom, taken together with the nitrogen atom, form an optionally substituted 4- to 8-membered heterocyclyl ring having, in addition to the nitrogen atom, 0-2 ring heteroatoms independently selected from N, O, and S;
R^{4y} is hydrogen, C₁₋₁₀ ar(C₁₋₄)alkyl, the aryl portion of which may be optionally substituted, an optionally substituted 5- or 6-membered aryl, heteroaryl, or heterocyclyl ring, or a C₁₋₄ alkyl or C₁₋₄ fluoroalkyl optionally substituted with one or two substituents independently selected from the group consisting of —OR^{5x}, —N(R^{4x})₂, —CO₂R^{5x}, or —C(O)N(R^{4x})₂; or
R^{4x} and R^{4y}, taken together with the nitrogen atom to which they are attached, form an optionally substituted 4- to 8-membered heterocyclyl ring having, in addition to the nitrogen atom, 0-2 ring heteroatoms independently selected from N, O, and S;
each R^{5x} independently is hydrogen, C₁₋₄ alkyl, C₁₋₄ fluoroalkyl, C₁₋₁₀ ar(C₁₋₄)alkyl, the aryl portion of which may be optionally substituted, or an optionally substituted 5- or 6-membered aryl, heteroaryl, or heterocyclyl ring;
each R^{5y} independently is hydrogen, an optionally substituted C₆₋₁₀ aryl, a C₆₋₁₀ar(C₁₋₄)alkyl, the aryl portion of which may be optionally substituted, or a C₁₋₄ alkyl or C₁₋₄ fluoroalkyl optionally substituted with one or two substituents independently selected from the group consisting of —OR^{5x}, —N(R^{4x})₂, —CO₂R^{5x}, or —C(O)N(Rex)₂; and
each R^{6x} independently is C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring.

27. The compound of claim 26, wherein Ring C is selected from the group consisting of: 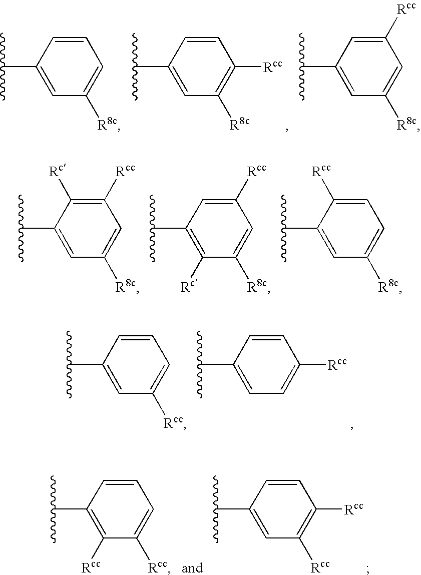
each R^{cc} independently is halo, —CN, —C(R^{5x})═C(R^{5x})(R^{1y}), —C≡C—R^{5y}, —OR^{5y}, —SR^{6x}, —N(R^{4x})(R^{4y}), —CO₂R^{5x}, —C(O)N(R^{4x})(R^{4y}), or a C₁₋₄ aliphatic or C₁₋₄ fluoroaliphatic optionally substituted with one or two substituents independently selected from the group consisting of —OR^{5x}, —N(R^{4x})(R^{4y}), —SR^{6x}, —CO₂R^{5x}, or —C(O)N(R^{4x})(R^{4y}); or two adjacent R^{cc}, taken together with the intervening ring atoms, form a fused Ring E;
R^{c′} is C₁₋₄ aliphatic, C₁₋₄ fluoroaliphatic, halo, —CN, —OH, —O(C₁₋₁₄ alkyl), —O(C₁₋₄ fluoroalkyl), —S(C₁₋₄ alkyl), —NH₂, —NH(C₁₋₄ alkyl), or —N(C₁₋₄ alkyl)₂; and
R^{8c} is C₁₋₄ aliphatic, C₁₋₄ fluoroaliphatic, or halo.

28. The compound of claim 27, wherein Ring C is selected from the group consisting of:

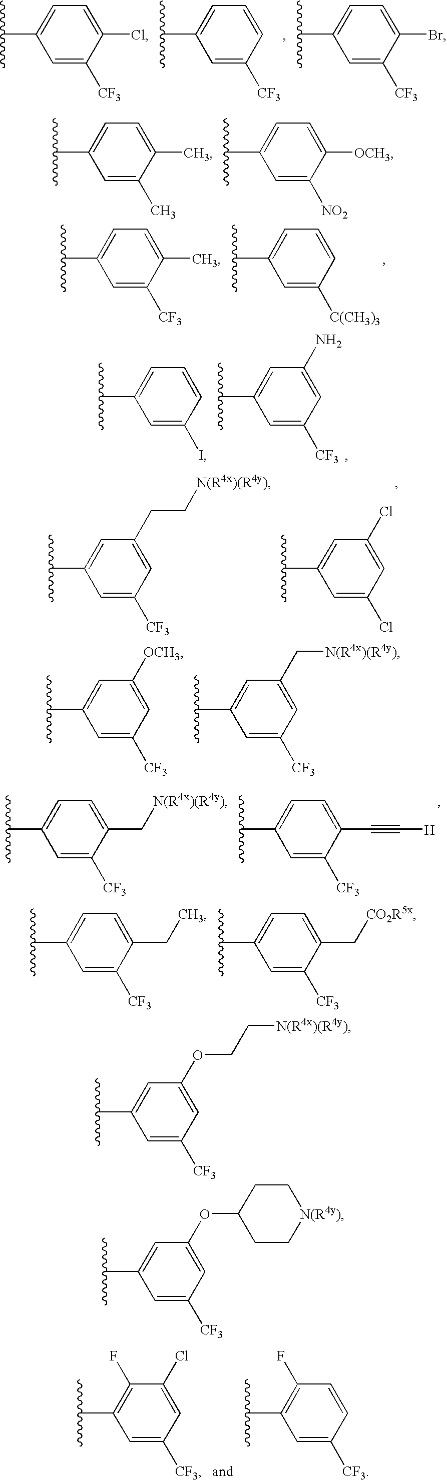

29. A compound of claim 2, having the formula (IV): 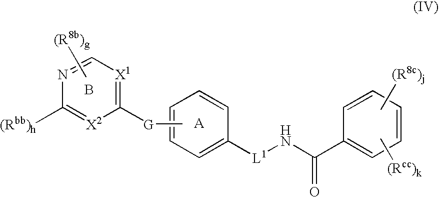
or a pharmaceutically acceptable salt thereof;
wherein:
G is —O— or —NH—;
X¹ and X² are each independently CH or N, provided that X¹ and X² are not both N;
one ring nitrogen atom in Ring B optionally is oxidized;
Ring A is substituted with 0-2 R^{aa};
each R^{aa} independently is —F, —Cl, —NO₂, —CH₃, —CF₃, —OCH—OCF₃, —SCH₃, —SO₂CH₃, —CN, —CO₂H, —C(O)NH₂, or —C(O)NHCH₃;
R^{bb} is selected from the group consisting of C₁₋₄ aliphatic, C₁₋₄ fluoroaliphatic, halo, —R^{2b}, -T¹-R^{1b}, T¹-R^{2b}, —V¹-T¹-R^{1b}, —V¹-T¹-R^{2b}, optionally substituted heteroaryl, and optionally substituted heterocyclyl;
T¹ is a C₁₋₆ alkylene chain optionally substituted with —F, C₁₋₃ alkyl, or C₁₋₃ fluoroalkyl, wherein the alkylene chain optionally is interrupted by —N(R⁴)—, —C(O)—N(R⁴)—, —C(═NR⁴)—N(R⁴)—, —N(R⁴)—C(O)—, or —N(R⁴)—C(═NR⁴)—;
V is —N(R⁴)—, —N(R⁴)C(O)—, —C(O)N(R⁴)—, —C(═NR⁴)—N(R⁴)—, —C(NR⁴)═N(R⁴)—, or —N(R⁴)—C(═NR⁴)—;
R^{1b} is an optionally substituted C₃₋₆ cycloaliphatic or an optionally substituted phenyl, pyrrolyl, imidazolyl, oxazolyl, thiazolyl, isoxazolyl, isothiazolyl, pyrazolyl, triazolyl, tetrazolyl, oxadiazolyl, thiadiazolyl, pyrrolinyl, imidazolinyl, pyrazolinyl, pyrrolidinyl, imidazolidinyl, pyrazolidinyl, piperidinyl, morpholinyl, piperazinyl, pyridyl, pyridazinyl, pyrimidinyl, pyrazinyl, or tetrahydropyrimidinyl ring;
R^{2b} is —OR⁵, —N(R⁴)₂, —NR⁴C(O)R⁵, —NR⁴C(O)N(R⁴)₂, —C(O)N(R⁴)—OR⁵, —C(O)N(R⁴)₂, —N(R⁴)—CO₂R⁵, —N(R⁴)—C(═NR⁴)—R⁵ or —C(═NR⁴)—N(R⁴)₂;
R^{1b} is selected from the group consisting of C₁₋₄ aliphatic, C₁₋₄ fluoroaliphatic, halo, —OH, —O(C₁₋₄ aliphatic), —NH₂, —NH(C₁₋₄ aliphatic), and —N(C₁₋₄ aliphatic)₂;
each R^{cc} independently is selected from the group consisting of C₁₋₄ aliphatic, C₁₋₄ fluoroaliphatic, halo, —R^{2c} and -T²-R^{2c}; or two adjacent R^{cc}, taken together with the intervening ring atoms, form a fused Ring E;
T² is a C₁₋₄ alkylene chain optionally substituted with one or two groups independently selected from —F, C₁₋₄ aliphatic, and C₁₋₄ fluoroaliphatic; and
each R^{2c} independently is —CN, —C(R⁵)═C(R⁵)₂, —C≡C—R⁵, —OR⁵, —SR⁶, —N(R⁴)₂, —NR⁴C(O)R⁵, —NR⁴C(O)N(R⁴)₂, —NR⁴CO₂R⁶, —CO₂R⁵, or —C(O)N(R⁴)₂;
each R^{8c} independently is selected from the group consisting of CIA aliphatic, C₁₋₄ fluoroaliphatic, —O(C₁₋₄ alkyl), —O(C₁₋₄ fluoroalkyl), and halo;
each R⁷ independently is an optionally substituted aryl or heteroaryl ring;
g is 0 or 1;
h is 0 or 1;
j is 0 or 1; and
k is 0, 1, or 2.

30. The compound of claim 29, wherein:
X and X² are each CH;
Ring A has no substituents R^{aa};
each R^{cc} independently is halo, —CN, —C(R^{5x})═C(R^{5x})(R^{5y}), —C≡C—R^{5y}, —OR^{5y}, —SR^{6x}, —CO₂R^{5x}, —C(O)N(R^{4x})(R^{4y}), or a C₁₋₄ aliphatic or C₁₋₄ fluoroaliphatic optionally substituted with one or two substituents independently selected from the group consisting of —OR^{5x}, —N(R^{4x})(R^{4y}), —SR^{6x}, —CO₂R^{5x}, or —C(O)N(R^{4x})(R^{4y});
R^{4x} is hydrogen, C₁₋₄ alkyl, C₁₋₄ fluoroalkyl, or C₆₋₁₀ ar(C₁₋₄)alkyl, the aryl portion of which may be optionally substituted, or two R^{4x} on the same nitrogen atom, taken together with the nitrogen atom, form an optionally substituted 4- to 8-membered heterocyclyl ring having, in addition to the nitrogen atom, 0-2 ring heteroatoms independently selected from N, O, and S;
R^{4y} is hydrogen, C₆₋₁₀ ar(C₁₋₄)alkyl, the aryl portion of which may be optionally substituted, an optionally substituted 5- or 6-membered aryl, heteroaryl, or heterocyclyl ring, or a C₁₋₄ alkyl or C₁₋₄ fluoroalkyl optionally substituted with one or two substituents independently selected from the group consisting of —OR^{5x}, —N(R^{4x})₂, —CO₂R^{x}, or —C(O)N(R^{4x})₂; or
R^{4x} and R^{4y}, taken together with the nitrogen atom to which they are attached, form an optionally substituted 4- to 8-membered heterocyclyl ring having, in addition to the nitrogen atom, 0-2 ring heteroatoms independently selected from N, O, and S;
each R^{5x} independently is hydrogen, C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring;
each R^{5y} independently is hydrogen, an optionally substituted C₆₋₁₀ aryl, a C₆₋₁₀ar(C₁₋₄)alkyl, the aryl portion of which may be optionally substituted, or a C₁₋₄ alkyl or C₁₋₄ fluoroalkyl optionally substituted with one or two substituents independently selected from the group consisting of —OR^{5x}, —N(R^{4x})₂, —CO₂R^{5x}, or —C(O)N(R^{4x})₂; and
each R^{6x} independently is C₁₋₄ alkyl, C₁₋₄ fluoroalkyl, C₆₋₁₀ ar(C₁₋₄)alkyl, the aryl portion of which may be optionally substituted, or an optionally substituted 5- or 6-membered aryl, heteroaryl, or heterocyclyl ring.

31. A compound selected from the compounds listed in Table 1.

32. A pharmaceutical composition comprising a compound according to claim 1 and a pharmaceutically acceptable carrier.

33. The pharmaceutical composition according to claim 32, formulated for administration to a human patient.

34. Use of a compound according to claim 1 for the treatment or prophylaxis of a human disorder.

35. The use according to claim 34, characterized in that the disorder is caused, mediated, or exacerbated by Raf kinase activity.

36. A method for the treatment of cancer in a patient in need thereof, comprising administering to the patient a compound of formula (I): 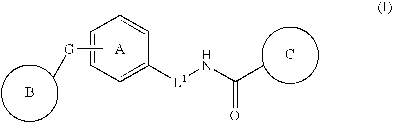
or a pharmaceutically acceptable salt thereof;
wherein:
G is —C(R^{d})(R^{e})—, —O—, —S, or —N(R^{f})—, wherein G is attached to Ring A at the position meta or para to L¹;
L is —[C(R^{g})(R^{h})]ₘ—C(R^{j})(R^{k})—;
Ring A is substituted with 0-2 R^{aa};
Ring B is a 5- or 6-membered heteroaryl ring having 1-3 ring nitrogen atoms and optionally one additional ring heteroatom selected from oxygen and sulfur;
Ring B is substituted on its substitutable ring carbon atoms with 0-2 R^{bb} and 0-2 R^{8b};
each R^{bb} independently is halo, —NO₂, —CN, —C(R⁵)═C(R⁷)₂, —C≡C—R⁵, —OR⁵, —SR⁶, —S(O)R⁶, —SO₂R⁶, —SO₂N(R⁴)₂, —N(R⁴)₂—NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —C(═NR⁴)—N(R⁴)—OR⁷, —C(R⁶)═N—OR⁵, or an optionally substituted aliphatic, aryl, heteroaryl, or heterocyclyl;
each R^{8b} independently is selected from the group consisting of C₁₋₄ aliphatic, C₁₋₄ fluoroaliphatic, halo, —OH, —O(C₁₋₄ aliphatic), —NH₂, —NH(C₁₋₄ alkyl), and —N(C₁₋₄ alkyl)₂;
each substitutable ring nitrogen atom in Ring B is unsubstituted or is substituted with —C(O)R⁵, —C(O)N(R⁴)₂, —CO₂R⁶, —SO₂R⁶, —SO₂N(R⁴)₂ C₁₋₄ aliphatic, an optionally substituted C₆₋₁₀ aryl, or a C₆₋₁₀ ar(C₁₋₄)alkyl, the aryl portion of which is optionally substituted;
one ring nitrogen atom in Ring B optionally is oxidized;
Ring C is a 5- or 6-membered aryl or heteroaryl ring having 0-3 ring nitrogen atoms and optionally one additional ring heteroatom selected from oxygen and sulfur;
Ring C is substituted on its substitutable ring carbon atoms with 0-2 R^{cc} and 0-2 R^{8c};
each R^{cc} independently is halo, —NO₂, —CN, —C(R⁵)═C(R⁵)₂, —C≡C—R⁵, —OR⁵, —SR⁶, —S(O)R⁶, —S_{—}₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —C(═NR⁴)—N(R⁴)—OR⁵, —C(R⁶)═N—OR⁵, or an optionally substituted aliphatic, aryl, heteroaryl, or heterocyclyl; or two adjacent R^{cc}, taken together with the intervening ring atoms, form a fused Ring E;
each R^{8c} independently is selected from the group consisting of C₁₋₄ aliphatic, C₁₋₄ fluoroaliphatic, —O(C₁₋₄ alkyl), —O(C₁₋₄ fluoroalkyl), and halo;
each substitutable ring nitrogen atom in Ring C is unsubstituted or is substituted with —C(O)R⁵, —C(O)N(R⁴)₂, —CO₂R⁶, —SO₂R⁶, —SO₂N(R⁴)₂, an optionally substituted C₆₋₁₀ aryl, or a C₁₋₄ aliphatic optionally substituted with —F, —OH, —O(C₁₋₄ alkyl), —CN, —N(R⁴)₂, —C(O)(C₁₋₄ alkyl), —CO₂H, —CO₂(C₁₋₄ alkyl), —C(O)NH₂, —C(O)NH(C₁₋₄ alkyl), or an optionally substituted C₆₋₁₀ aryl ring;
one ring nitrogen atom in Ring C optionally is oxidized;
Ring E is a 5- or 6-membered aromatic or non-aromatic ring having 0-3 ring heteroatoms independently selected from the group consisting of O, N, and S;
each substitutable saturated ring carbon atom in Ring E is unsubstituted or is substituted with ═O, ═S, ═C(R⁵)₂, or —R^{ee};
each substitutable unsaturated ring carbon atom in Ring E is unsubstituted or is substituted with —R^{ee};
each R^{ee} independently is halo, —NO₂, —CN, —C(R⁵)═C(R⁵)₂, —C≡C—R⁵, —OR⁵, —SR⁶, —S(O)R⁶, —SO₂R⁶, —SO₂N(R⁴)₂, —N(R⁴)₂, —NR⁴C(O)R⁵, —NR⁴C(O)N(R⁴)₂, —N(R⁴)C(═NR⁴)—N(R⁴)₂, —N(R⁴)C(═NR⁴)—R⁶, —NR⁴CO₂R⁶, —N(R⁴)SO₂R⁶, —N(R⁴)SO₂N(R⁴)₂, —O—C(O)R⁵, —OC(O)N(R⁴)₂, —C(O)R⁵, —CO₂R⁵, —C(O)N(R⁴)₂, —C(O)N(R⁴)—OR⁵, —C(O)N(R⁴)C(═NR⁴)—N(R⁴)₂, —N(R⁴)C(═NR⁴)—N(R⁴)—C(O)R⁵, —C(═NR⁴)—N(R⁴)₂, —C(═NR⁴)—OR⁵, —C(═NR⁴)—N(R⁴)—OR⁵, —C(R⁶)═N—OR⁵, or an optionally substituted C₁₋₆ aliphatic;
each substitutable ring nitrogen atom in Ring E is unsubstituted or is substituted with —C(O)R⁵, —C(O)N(R⁴)₂, —CO₂R⁶, —SO₂R⁶, —SO₂N(R⁴)₂, C₁₋₄ aliphatic, an optionally substituted C₆₋₁₀ aryl, or a C₆₋₁₀ ar(C₁₋₄)alkyl, the aryl portion of which is optionally substituted;
one ring nitrogen or sulfur atom in Ring E optionally is oxidized;
R^{aa} is halo, —NO₂, —CN, —OR⁵, —SR⁶, —S(O)R⁶, —SO₂R⁶, —SO₂N(R⁴)₂, —N(R⁴)₂, —OC(O)R⁵, —CO₂R⁵, —C(O)N(R⁴)₂, —N(R⁴)SO₂R⁶, —N(R⁴)SO₂N(R⁴)₂, or a C₁₋₄ aliphatic or C₁₋₄ fluoroaliphatic optionally substituted with —OR⁵ or —N(R⁴)₂, provided that no more than one R^{aa} is —OH;
R^{d} is hydrogen, fluoro, C₁₋₄ aliphatic, C₁₋₄ fluoroaliphatic, —NH₂, —NH(C₁₋₄ alkyl), —N(C₁₋₄ alkyl)₂, OH, or —O(C₁₋₄ alkyl);
R^{e} is hydrogen, fluoro, C₁₋₄ aliphatic, or C₁₋₄ fluoroaliphatic; or R^{d} and R^{e}, taken together with the carbon atom to which they are attached, form a 3- to 6-membered cycloaliphatic or heterocyclyl ring;
R^{f} is —H, —C(O)R⁵, —C(O)N(R⁴)₂, —CO₂R⁶, —SO₂R⁶, —SO₂N(R⁴)₂, or an optionally substituted C₁₋₆ aliphatic;
R^{g} is hydrogen, fluoro, C₁₋₄ aliphatic, or C₁₋₄ fluoroaliphatic, and R^{h} is hydrogen, fluoro, C₁₋₄ aliphatic, C₁₋₄ fluoroaliphatic, —OH, —O(C₁₋₄ alkyl), —N(R⁴)₂, —N(R⁴)C(O)(C₁₋₄ aliphatic); or R^{g} and R^{h}, taken together with the carbon atom to which they are attached, form a 3- to 6-membered cycloaliphatic ring;
R^{j} is hydrogen, fluoro, C₁₋₄ aliphatic, or C₁₋₄ fluoroaliphatic, and R^{k} is hydrogen, fluoro, C₁₋₄ aliphatic, C₁₋₄ fluoroaliphatic, —C(O)(C₁₋₄ alkyl), —CO₂H, —CO₂(C₁₋₄ alkyl), or —C(O)N(R⁴)₂; or R^{j} and R^{k}, taken together with the carbon atom to which they are attached, form a 3- to 6-membered cycloaliphatic ring; or
R_{g} and R^{j} are each hydrogen, fluoro, C₁₋₄ aliphatic, or C₁₋₄ fluoroaliphatic, and R^{h} and R^{k}, taken together with the intervening carbon atoms, form a 3- to 6-membered cycloaliphatic ring;
each R⁴ independently is hydrogen or an optionally substituted aliphatic, aryl, heteroaryl, or heterocyclyl group; or two R⁴ on the same nitrogen atom, taken together with the nitrogen atom, form an optionally substituted 4- to 8-membered heterocyclyl ring having, in addition to the nitrogen atom, 0-2 ring heteroatoms independently selected from N, O, and S;
each R⁵ independently is hydrogen or an optionally substituted aliphatic, aryl, heteroaryl, or heterocyclyl group; and
each R⁶ independently is an optionally substituted aliphatic, aryl, or heteroaryl group; and
m is 1 or 2.
